(19) United States
(12) Patent Application Publication
(10) Pub. No.: US 2013/0065897 A1
(43) Pub. Date: Mar. 14, 2013

(54) COMPOUNDS, PREPARATION AND USES THEREOF

(71) Applicants: Julie Ann Spicer, Auckland (NZ); Kristiina Maria Huttunen, Kuopio (FI); Dani Michelle Lyons, Austin, TX (US); Joseph Albert Trapani, Camberwell (AU); Mark John Smyth, Clifton Hill (AU); William Alexander Denny, Manukai (NZ)

(72) Inventors: Julie Ann Spicer, Auckland (NZ); Kristiina Maria Huttunen, Kuopio (FI); Dani Michelle Lyons, Austin, TX (US); Joseph Albert Trapani, Camberwell (AU); Mark John Smyth, Clifton Hill (AU); William Alexander Denny, Manukai (NZ)

(73) Assignee: PETER MACCALLUM CANCER INSTITUTE, East Melbourne, Victoria (AU)

(21) Appl. No.: 13/519,096

(22) PCT Filed: Dec. 22, 2010
(86) PCT No.: PCT/AU2010/001731

§ 371 Date: Nov. 19, 2012

Related U.S. Application Data
(60) Provisional application No. 61/289,871, filed on Dec. 23, 2009.

(51) Int. Cl. A61K 31/4178; C07D 405/14; C07D 409/14; C07D 405/10; A61K 31/4439; A61K 31/4709; C07D 409/06; C07D 413/14; A61K 31/5377; A61K 31/422; C07D 417/14; A61K 31/427; A61K 31/496; A61K 31/454; A61K 31/4725; A61P 31/12; A61P 3/10; A61P 37/06; C12N 5/071

(52) U.S. Cl. 514/235.2; 435/375; 548/311.4; 514/389; 546/274.4; 514/337; 548/312.1; 546/173; 514/314; 548/315.1; 544/139; 514/235.8; 548/235; 514/374; 548/204; 514/365; 548/247; 514/378; 548/214; 514/372; 544/370; 514/254.07; 546/200; 514/323; 546/141; 514/309

(57) ABSTRACT

The present invention provides novel compounds of the Formula I, pharmaceutical compositions comprising such compounds and methods for using such compounds as agents or drugs for inhibiting perforin activity and for treating a subject at risk of or susceptible to a disease or disorder, or having a disease or disorder associated with undesirable perforin activity.

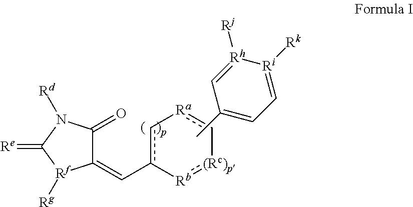

The present invention relates generally to compounds capable of modulating perforin activity, more particularly to compounds capable of inhibiting perforin activity, and uses thereof. More specifically, the present invention relates to benzylidene-2-thioxoimidazolidinones and related compounds and analogues thereof, to their preparation, and to their use as tools for biological studies or as agents or drugs for immunosuppressive therapies, whether they are used alone or in combination with other treatment modalities.

### BACKGROUND

Cytotoxic T lymphocytes (CTL) and natural killer (NK) cells perform tumour surveillance and provide a defence against viral infection and intracellular pathogens, by inducing apoptosis of virus-infected or transformed cells. A major component of this defence is the glycoprotein perforin. Upon stable conjugation of the CTL or NK cell with a target cell, perforin is released, binds calcium and assembles into aggregates of 12-18 molecules that form trans-membrane pores in the plasma membrane. This allows leakage of cell contents and the entry of secreted serine proteases (granzymes) which promote apoptosis.

Stimulation of CTL and NK cells, leading to abnormal cellular destruction, occurs in several autoimmune diseases (e.g., insulin-dependent diabetes) and in therapy-induced conditions (e.g., allograft rejection, graft-versus-host disease). In this context, small-molecule inhibitors of perforin function are of potential interest as a new class of therapeutic immunosuppressive agents.

To date, the only reported inhibitors of perforin function are non-selective, complex natural products, primarily concanamycin A and other V-ATPase inhibitors such as bafilomycin A and prodigiosin 25-Cs that inhibit acidification. Other reported non-selective perforin inhibitors include cytochalasin D (an inhibitor of actin polymerisation), antimycin A and oligomycin A (inhibitors of cell respiration) and some protein kinase inhibitors (calphostin C, herbimycin A, staurosporine). However, such non-selective compounds display a broad spectrum of biological effects that generally make them undesirable for use in the treatment or prevention of conditions associated with aberrant perforin expression and/or activity.

In one or more aspects, the present invention may advantageously provide a class of compounds and their analogues as drugs for immunosuppressive therapies, or to at least provide a useful alternative to existing treatment modalities.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date.

### SUMMARY OF THE INVENTION

The present invention relates to a method of inhibiting the activity of a perforin molecule, or a fragment or variant thereof, on a cell. The said method comprises exposing the cell to a benzylidene-2-thioxoimidazolidinone compound or a derivative thereof or a salt thereof.

One embodiment of the present invention provides a method of inhibiting activity of a perforin molecule, or a fragment or variant thereof, on a cell, said method comprising exposing the cell to a compound of the Formula (I):

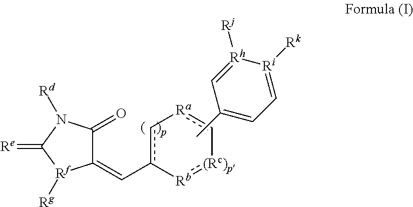

wherein:

R^{a}, R^{b} and R^{c} are independently C, N, S or O;

p and p′ are independently 0 or 1, provided that at least one of them is 1;

R^{d} is H or methyl;

R^{e} is O, S, NH or absent;

R^{f} is C or N;

R^{g} is H or methyl;

R^{h} and Rⁱ are independently C or N;

R^{j} and R^{k} are independently H, F, Cl, Br, CN, Me, OMe, pyridyl, OH, SMe, acetyl, CH₂OAc, COOMe, COOEt, NHAc, NHSO₂Me, SO₂Me, SO₂NH₂, CONH₂, CONHJ or CONJJ;

or

R^{j} and R^{k} combined are a 5-membered or 6-membered ring of the formula —C(O)—Z—(R^{l}R^{m})ₙ— where n=1 or 2. Z is O, CH₂, NJ or NC(O)J, R^{l} and R^{m} are independently H, C₁-C₆ alkyl or cyclopropyl; or

—C(O)—Z—O—, where Z is NJ or NC(O)J; or

—OCO—; and

J is H or C₁-C₆ alkyl optionally substituted with acyl, hydroxyl, amino, alkylamino, alkenylamino, cycloalkylamino, cycloalkenylamino, arylamino, heteroaryl, heteroarylamino, heterocyclyl, heterocyclylamino, aminoarylamino, aminoheteroarylamino, aminoheterocyclylamino, alkylheterocyclyl, heteroarylcarbonylamino, heterocyclylcarbonylamino, alkyloxy, alkenyloxy, alkynyloxy, cycloalkyloxy, cycloalkenyloxy, aryloxy, heteroaryloxy, heterocyclyloxy, aminoalkyloxy, aminoalkenyloxy, aminoalkynyloxy, aminocycloalkyloxy; aminocycloalkenyloxy, aminoaryloxy, or aminoheteroaryloxy; or pharmaceutically acceptable salts, solvates, hydrates, and prodrugs thereof.

A further embodiment provides a method of inhibiting activity of a perforin molecule, or a fragment or variant thereof, on a cell, said method comprising exposing the cell to a compound of the Formula (Ia):

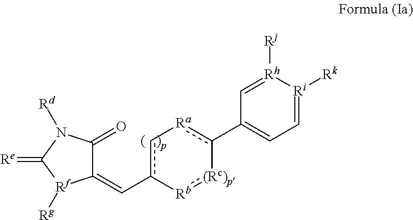

wherein:

R^{a}, R^{b} and R^{c} are independently C, N, S or O;

p and p′ are independently 0 or 1, provided that at least one of them is 1;

R^{d} is H or methyl;

R^{e} is O, S, NH or absent;

R^{f} is C or N;

R^{g} is H or methyl;

R^{h} and Rⁱ are independently C or N;

R^{j} and R^{k} are independently H, F, Cl, Br, CN, Me, OMe, pyridyl, OH, SMe, acetyl, CH₂OAc, COOMe, COOEt, NHAc, NHSO₂Me, SO₂Me, SO₂NH₂, CONH₂, CONHJ or CONJJ;

or

R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or

—C(O)—Z—O—, where Z is NJ or NC(O)J; or

—OCO—; and

J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof.

A further embodiment provides a method of inhibiting activity of a perforin molecule, or fragment or variant thereof, on a cell, said method comprising exposing the cell to a compound of the Formula (II):

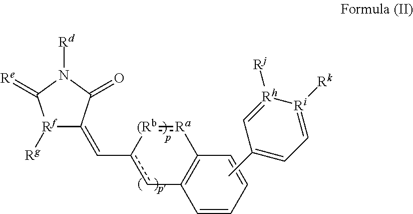

wherein:

R^{a} and R^{h} are independently C, N, S or O;

p and p′ are independently 0 or 1, provided that at least one of them is 1;

R^{d} is H or methyl;

R^{e} is O, S, NH or absent;

R^{f} is C or N;

R^{g} is H or methyl;

R^{h} and Rⁱ are independently C or N;

R^{j} and R^{k} are independently H, F, Cl, Br, CN, Me, OMe, pyridyl, OH, SMe, acetyl, CH₂OAc, COOMe, COOEt, NHAc, NHSO₂Me, SO₂Me, SO₂NH₂, CONH₂, CONHJ or CONJJ;

or

R^{j} and R^{k} combined are a 5-membered or 6-membered ring of the formula —C(O)—Z—(CR^{l}R^{m})ₙ— where n=1 or 2, Z is O, CH₂, NJ or NC(O)J, R¹ and R² are independently H, C₁-C₆ alkyl or cyclopropyl; or

—C(O)—Z—O—, where Z is NJ or NC(O)J; or

—COC—; and

J is H or C₁-C₆ alkyl optionally substituted, with acyl, hydroxyl, amino, alkylamino, alkenylamino, cycloalkylamino, cycloalkenylamino, arylamino, heteroaryl, heteroarylamino, heterocyclyl, heterocyclylamino, aminoarylamino, aminoheteroarylamino, aminoheterocyclylamino, alkylheterocyclyl, heteroarylcarbonylamino, heterocyclylcarbonylamino, alkyloxy, alkenyloxy, alkynyloxy, cycloalkyloxy, cycloalkenyloxy, aryloxy, heteroaryloxy, heterocyclyloxy, aminoalkyloxy, aminoalkenyloxy, aminoalkynyloxy, aminocycloalkyloxy, aminocycloalkenyloxy, aminoaryloxy, or aminoheteroaryloxy; or pharmaceutically acceptable salts, solvates, hydrates, and prodrugs thereof.

In another aspect, the present invention provides a compound of the formula (I):

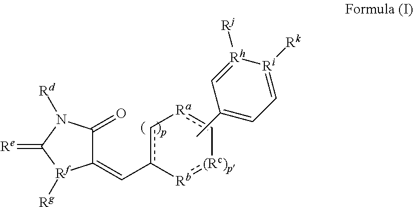

wherein:

R^{a}, R^{b} and R^{c} are independently C, N, S or O;

p and p′ are independently 0 or 1, provided that at least one of them is 1;

R^{d} is H or methyl;

R^{e} is O, S, NH or absent;

R^{f} is C or N;

R^{g} is H or methyl;

R^{h} and Rⁱ are independently C or N;

R^{j} and R^{k} are independently H, F, Cl, Br, CN, OMe, pyridyl, OH, SMe, acetyl, CH₂OAc, COOMe, COOEt, NHAc, NHSO₂Me, SO₂Me, SO₂NH₂, CONH₂, CONHJ or CONJJ, provided that when R^{j} is H R^{k} is not H;

or

R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or

—C(O)—Z—O—, where Z is NJ or NC(O)J; and

J is H or C₁-C₆ alkyl optionally substituted with acyl, hydroxyl, amino, alkylamino, alkenylamino, cycloalkylamino, cycloalkenylamino, arylamino, heteroaryl, heteroarylamino, heterocyclyl, heterocyclylamino, aminoarylamino, aminoheteroarylamino, aminoheterocyclylamino, alkylheterocyclyl, heteroarylcarbonylamino, heterocyclylcarbonylamino, alkyloxy, alkenyloxy, alkynyloxy, cycloalkyloxy, cycloalkenyloxy, aryloxy, heteroaryloxy, heterocyclyloxy, aminoalkyloxy, aminoalkenyloxy, aminoalkynyloxy, aminocycloalkyloxy, aminocycloalkenyloxy, aminoaryloxy, or aminoheteroaryloxy; or pharmaceutically acceptable salts, solvates, hydrates, and prodrugs thereof; provided that when R^{j} and R^{k} combined are a 5-membered ring of the formula —C(O)—Z—(CR^{l}R^{m})ₙ—, where n=1, Z is O and R^{l} and R^{m} are both H, R^{a}, R^{b} or R^{c} are not O.

In a further aspect the present invention provides a compound of the formula (I) in the form of formula (Ia):

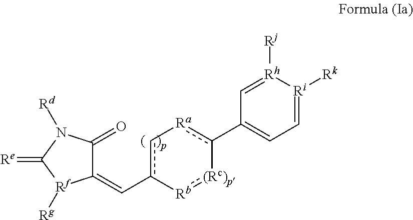

wherein:

R^{a}, R^{b} and R^{c} are independently C, N, S or O;

p and p′ are independently 0 or 1, provided that at least one of them is 1;

R^{d} is H or methyl;

R^{e} is O, S, NH or absent;

R^{f} is C or N;

R^{g} is H or methyl;

R^{h} and Rⁱ are independently C or N;

R^{j} and R^{k} are independently H, F, Cl, Br, CN, OMe, pyridyl, OH, SMe, acetyl, CH₂OAc, COOMe, COOEt, NHAc, NHSO₂Me, SO₂Me, SO₂NH₂, CONH₂, CONHJ or CONJJ, provided that when R¹ is H, R² is not H;

or

R^{j} and R^{k} combined are a 5-membered or 6-membered ring of the formula —C(O)—Z—(CR^{l}R^{m})— where n=1 or 2, Z is O, CH₂, NJ or NC(O)J, R^{l} and R^{m} are independently H, C₁-C₆ alkyl or cyclopropyl; or

—C(O)—Z—O—, where Z is NJ or NC(O)J; and

J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof; provided that when R^{j} and R^{k} combined are a 5-membered ring of the formula —C(O)—Z—(CR^{l}R^{m})ₙ—, where n=1, Z is O and R^{l} and R^{m} are both H, R^{a}, R^{b} or R^{c} are not O.

A preferred embodiment of compounds of the Formula Ia is wherein:

R^{a} and R^{b} are independently C, N or S;

R^{c} is C;

R^{d} is H;

R^{e} is O or S;

R^{f} is N;

R^{g} is H;

R^{h} and Rⁱ are C;

R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or

—C(O)—Z—O—, where Z is NJ or NC(O)J; and

J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof.

In another aspect of the present invention, there is provided a pharmaceutical composition including the compound according to the present invention (as herein described) and a pharmaceutically acceptable carrier, excipient, diluent and/or adjuvant.

In another aspect of the present invention, there is provided a method of inhibiting activity of a perforin molecule, or a fragment or variant thereof, on a cell, said method comprising exposing the cell to a compound, or a pharmaceutically acceptable salt or a derivative thereof, as herein described.

In another aspect of the present invention, there is provided a pharmaceutical composition comprising a compound, or a pharmaceutically acceptable salt or a derivative thereof, as herein described, preferably together with a pharmaceutically acceptable carrier, excipient, diluent and/or adjuvant.

In yet a further aspect of the present invention, there is provided a prophylactic or therapeutic method of treating a subject at risk of or susceptible to a disease or disorder, or having a disease or disorder associated with undesirable perforin activity, said method comprising administering to said subject a compound, or a pharmaceutically acceptable salt or a derivative thereof, as herein described. Preferably, the disease or disorder is juvenile diabetes mellitus (type 1 or insulin dependent), graft-versus-host disease, chronic or acute allograft rejection or a disorder associated with cytotoxic T lymphocyte-mediated immune pathology, for example perforin-induced immune pathology associated with various virus infections.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### DETAILED DESCRIPTION OF THE INVENTION

Despite evidence of its apparent role in the aforementioned pathophysiologies, the biological function of perforin remains poorly understood at the molecular and cellular levels. This lack of substantial progress has been mostly attributed to a lack of cell lines capable of synthesising and storing this toxic protein for the purposes of further investigation. In this regard, the present inventors have previously provided a method of expressing sufficient quantities of recombinant perforin in a cell, or fragment or variants thereof, which avoid the undesirable effects attributed to perforin's inherent cytotoxicity (see WO 2005/083098, the entire contents of which are incorporated herein by reference). By utilising this methodology, the present inventors have for the first time, been able to isolate recombinant perforin in sufficient quantities that allow for the screening of compounds that modulate perforin expression and/or activity. Using such screening methods, the present inventors have now identified compounds that are capable of inhibiting perforin activity, providing a means of treating or preventing diseases or disorders associated with aberrant perforin expression and/or activity, or disorders where CTL, NK cells or other lymphocytes pathologically target tissues through the use of perforin or perforin-dependent pathways.

In this specification a number of terms are used which are well known to a skilled addressee. Nevertheless for the purposes of clarity a number of terms will be defined.

Reference throughout the specification and the accompanying claims to benzylidene-2-thioxoimidazolidinone compounds or derivatives thereof encompasses all of the compounds disclosed herein of the formula I, formula Ia or formula II. Derivatives thereof include but are not limited to those compounds, for example, in which the benzylidene substituent is substituted with a furylmethylene (R^{a}, R^{b} or R^{c}=O, and p and p′=1 and 0) or thienylmethylene (R^{a}, R^{b} or R^{c}=S, and p and p′=1 and 0) substituent, and those compounds in which the thioxoimidazolidinone substituent is substituted for imidiazolidinedione (R^{e}=O, R^{f}=N, R^{g}=H), aminodihydroimidazolone (R^{e}=NH, R^{f}=N, R^{g}=H) or pyrrolidinedione (R^{e}=O, R^{f}=C, R^{g}=H).

As used herein, the term “unsubstituted” means that there is no substituent or that the only substituents are hydrogen.

The term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, hydroxyl, amino, alkylamino, alkenylamino, cycloalkylamino, cycloalkenylamino, arylamino, heteroarylamino, heterocyclylamino, aminoarylamino, aminoheteroarylamino, aminoheterocyclylamino, tetrahydropyridinylamino, azetidinyl, pyrrolidinyl, piperidinyl, piperazinyl, morpholinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkoxy, alkenyloxy, alkynyloxy, cycloalkyloxy, cycloalkenyloxy, aryloxy, heteroaryloxy, heterocyclyloxy, aminoalkoxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy, or piperazinyloxy

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Acyl” means an R—C(═O)— group in which the R group may be an alkyl, cycloalkyl, heterocycloalkyl, aryl or heteroaryl group as defined herein. Examples of acyl include acetyl and benzoyl. The group is bonded to the remainder of the molecule through the carbonyl carbon.

“Alkyl” as a group or, part of a group refers to a straight or branched aliphatic hydrocarbon group, preferably a C₁-C₁₀ alkyl, more preferably a C₁-C₈ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkenyl” means an aliphatic hydrocarbon group containing at least one carbon-carbon doublebond and which may be straight or branched preferably having from 2-10 carbon atoms, more preferably 2-8 carbon atoms, more preferably 2-6 carbon atoms in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl.

“Alkynyl” means an aliphatic hydrocarbon group containing a carbon-carbon triple bond and which may be straight or branched preferably having from 2-10 carbon atoms, more preferably 2-8 carbon atoms, more preferably 2-6 carbon atoms in the normal chain. “Alkylamino” includes both mono-alkylamino and dialkylamino, unless specified. “Mono-alkylamino” means an Alkyl-NH— group, in which alkyl is as defined herein. “N,N-dialkylamino” means a (alkyl)₂N— group, in which each alkyl may be the same or different and are each as defined herein for alkyl. The alkyl group is preferably a C₁-C₁₀ alkyl group. The group is bonded to the remainder of the molecule through the nitrogen atom.

“Alkenylamino” includes both mono-alkylamino and dialkylamino, unless specified. “Mono-alkylamino” means an Alkenyl-NH— group, in which alkenyl is as defined herein. The alkenyl group is preferably a C₂-C₁₀ alkyl group. The group is bonded to the remainder of the molecule through the nitrogen atom.

“Alkyloxy” as a group or part of a group refers to an alkyl-O— group in which alkyl is as defined herein. Preferably the alkyloxy is a C₁-C₁₀alkyloxy. Examples include, but are not limited to, methoxy and ethoxy. The group may be a terminal group or a bridging group. If the group is a terminal group it is bonded to the remainder of the molecule through the oxygen atom.

“Aminoalkyloxy” refers to an alkyloxy group as defined herein, further substituted with at least one amine. Preferred aminoalkyloxy groups are C₁-C₁₀ aminoalkyloxy groups. The group may be a terminal group or a bridging group. If the group is a terminal group it is bonded to the remainder of the molecule through the oxygen atom.

“Alkenyloxy” refers to an alkenyl-O— group in which alkenyl is as defined herein. Preferred alkenyloxy groups are C₂-C₁₀ alkenyloxy groups. The group is bonded to the remainder of the molecule through the oxygen atom.

“Aminoalkenyloxy” refers to an alkenyloxy group as defined herein, further substituted with at least one amine. Preferred aminoalkenyloxy groups are C₂-C₁₀ aminoalkenyloxy groups. The group is bonded to the remainder of the molecule through the oxygen atom.

“Alkynyloxy” refers to an alkynyl-O— group in which alkynyl is as defined herein. Preferred alkynyloxy groups are C₂-C₁₀ alkynyloxy groups. The group is bonded to the remainder of the molecule through the oxygen atom.

“Aminoalkynyloxy” refers to an alkynyloxy group as defined herein, further substituted with, at least one amine. Preferred aminoalkynyloxy groups are C₂-C₁₀ aminoalkynyloxy groups. The group is bonded to the remainder of the molecule through the oxygen atom.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₀ aryl group.

“Arylamino” includes both mono-arylamino and di-arylamino unless specified. Mono-arylamino means a group of formula aryl-NH—, in which aryl is as defined herein. “N,N-diarylamino” means a group of formula (aryl)₂N— where each aryl may be the same or different and are each as defined herein for aryl. The group is bonded to the remainder of the molecule through the nitrogen atom.

“Aminoarylamino” refers to a group of formula (NH₂)ₙ-aryl-NH—, in which arylamino is as defined herein, further substituted with at least one amine at the ortho-, meta- or para position. The group is bonded to the remainder of the molecule through the nitrogen atom.

“Aryloxy” refers to an aryl-O— group in which the aryl is as defined herein. Preferably the aryloxy is a C₆-C₁₀aryloxy. The group is bonded to the remainder of the molecule through the oxygen atom.

“Aminoaryloxy” refers to a group of formula (NH₂)ₙ-aryl-O—, in which aryloxy is as defined herein, further substituted with at least one amine at the ortho-, meta- or para position. The group is bonded to the remainder of the molecule through the oxygen atom.

“Cycloalkyl” refers to a saturated monocyclic or fused or spiro polycyclic, carbocycle preferably containing from 3 to 10 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane.

“Cycloalkenyl” refers to a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups.

“Cycloalkylamino” refers to a cycloalkyl-NH— group in which cycloalkyl is as defined herein: Preferably the cycloalkylthio is a C₃-C₁₀cycloalkylthio. The group is bonded to the remainder of the molecule through the nitrogen atom.

“Cycloalkenylamino” refers to a cycloalkenyl-NH— group in which the cycloalkenyl is as defined herein. Preferably the cycloalkenyloxy is a C₃-C₁₀cycloalkenyloxy. The group is bonded to the remainder of the molecule through the nitrogen atom.

“Cycloalkyloxy” refers to a cycloalkyl-O— group in which cycloalkyl is as defined herein. Preferably the cycloalkyloxy is a C₃-C₁₀cycloalkyloxy. Examples include, but are not limited to, cyclopropanoxy and cyclobutanoxy. The group is bonded to the remainder of the molecule through the oxygen atom.

“Aminocycloalkyloxy” refers to a cycloalkyloxy group is as defined herein, further substituted on one or more of the available carbon atoms with at least one amine. Preferably the aminocycloalkyloxy is a C₃-C₁₀ aminocycloalkyloxy. The group is bonded to the remainder of the molecule through the oxygen atom.

“Cycloalkenyloxy” refers to a cycloalkyloxy group defined herein containing at least one carbon-carbon double bond.

“Aminocycloalkenyloxy” refers to an aminocycloalkyloxy group defined herein containing at least one carbon-carbon double bond.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

“Heteroarylamino” refers to a heteroaryl-NH— group in which the heteroaryl is as defined herein. Preferably the heteroarylamino is a C₂-C₁₀heteroarylamino. The group is bonded to the remainder of the molecule through the amino atom.

“Aminoheteroarylamino” refers to a (NH₂)ₙ-heteroaryl-NH— group in which the heteroarylamino is as defined herein, further substituted at one or more of the ring members with at least one amine. Preferably the aminoheteroarylamino is a C₂-C₁₀ aminoheteroarylamino. The group is bonded to the remainder of the molecule through the amino atom.

“Heteroaryloxy” refers to a heteroaryl-O— group in which the heteroaryl is as defined herein. Preferably the heteroaryloxy is a C₂-C₁₀heteroaryloxy. The group is bonded to the remainder of the molecule through the oxygen atom.

“Aminoheteroaryloxy” refers to a (NH₂)ₙ-heteroaryl-O— group in which the heteroaryloxy is as defined herein, further substituted at one or more of the ring members with at least one amine. Preferably the aminoheteroaryloxy is a C₂-C₁₀ aminoheteroarylamino. The group is bonded to the remainder of the molecule through the oxygen atom.

“Heteroarylcarbonylamino” refers to a heteroaryl-C(O)—NH— group in which the heteroaryl is as defined herein. Preferably the Heteroarylcarbonylamino is a C₂-C₁₀ Heteroarylcarbonylamino. The group is bonded to the remainder of the molecule through the amino atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocyclyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocyclyl substituents include azetidinyl, pyrrolidyl, tetrahydrofuryl, tetrahydrothiofuranyl, piperidinyl, piperazinyl, tetrahydropyridinyl, morpholino, 1,3-diazapane, 1,4-diazapane, 1,4-oxazepane, and 1,4-oxathiapane.

“Heterocyclyloxy” refers to a heterocyclyl-O— group in which the heterocycloalkyl is as defined herein. Preferably the heterocyclyloxy is a C₂-C₁₀ heterocyclyloxy. The group is bonded to the remainder of the molecule through the oxygen atom.

“Heterocyclylamino” refers to a heterocyclyl-NH— group in which the heterocycloalkyl is as defined herein. Preferably the heterocyclylamino is a C₂-C₁₀ heterocyclylamino. The group is bonded to the remainder of the molecule through the nitrogen atom.

“Aminoheterocyclylamino” refers to a NH₂-heterocyclyl-NH— group in which the heterocycloalkylamino is as defined herein, further substituted with an amine at one of the ring members. Preferably the aminoheterocyclylamino is a C₂-C₁₀ aminoheterocyclylamino. The group is bonded to the remainder of the molecule through the nitrogen atom.

“Heterocyclylcarbonylamino” refers to a heterocyclyl-C(O)—NH— group in which the heterocyclyl is as defined herein. Preferably the Heterocyclylcarbonylamino is a C₂-C₁₀ Heterocyclylcarbonylamino. The group is bonded to the remainder of the molecule through the amino atom. Examples of suitable heterocyclylcarbonylamino substituents include azetidinylcarbonylamino, piperidinylcarbonylamino and piperazinylcarbonylamino.

“Alkylheterocyclyl” refers to an alkyl-heterocyclyl- group in which alkyl and heterocyclyl groups are as defined herein. Preferably the alkyl is a C₁-C₆alkyl group bound to the heterocyclyl group via either a carbon or heteroatom. The heterocyclic ring is preferably from 3 to 10 membered, more preferably 4 to 7 membered. The group is bonded to the remainder of the molecule via one of the ring-member atoms.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and for diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

In one embodiment the compounds of the present invention are selected from, but not limited to the group consisting of:

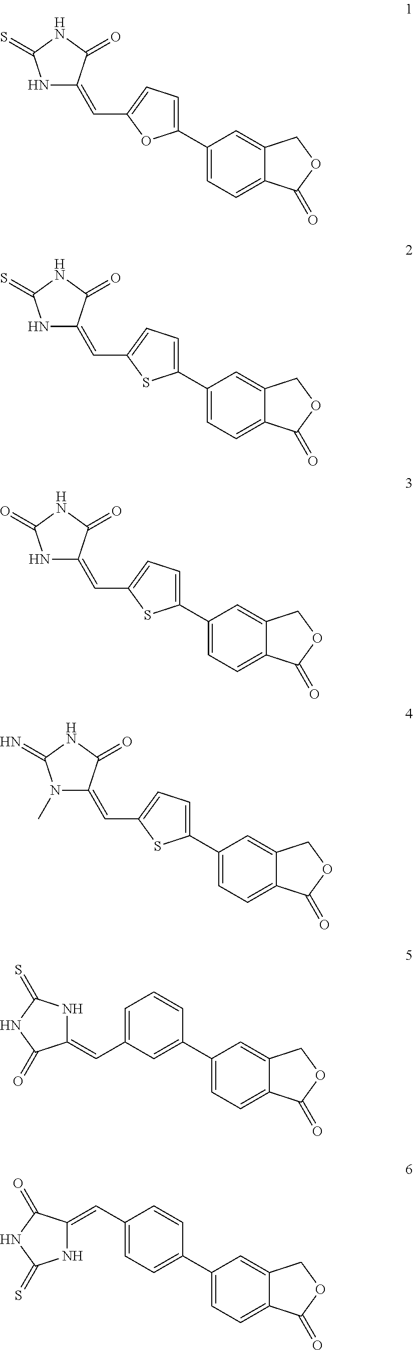 [SMILES: O=C1NC(=S)N/C1=C/c1ccc(-c2ccc3c(c2)COC3=O)o1 ; O=C1NC(=S)N/C1=C/c1ccc(-c2ccc3c(c2)COC3=O)s1 ; O=C1NC(=O)/C(=C\c2ccc(-c3ccc4c(c3)COC4=O)s2)N1 ; CN1C(=N)NC(=O)/C1=C\c1ccc(-c2ccc3c(c2)COC3=O)s1 ; O=C1NC(=S)N/C1=C\c1cccc(-c2ccc3c(c2)COC3=O)c1 ; O=C1NC(=S)N/C1=C\c1ccc(-c2ccc3c(c2)COC3=O)cc1]

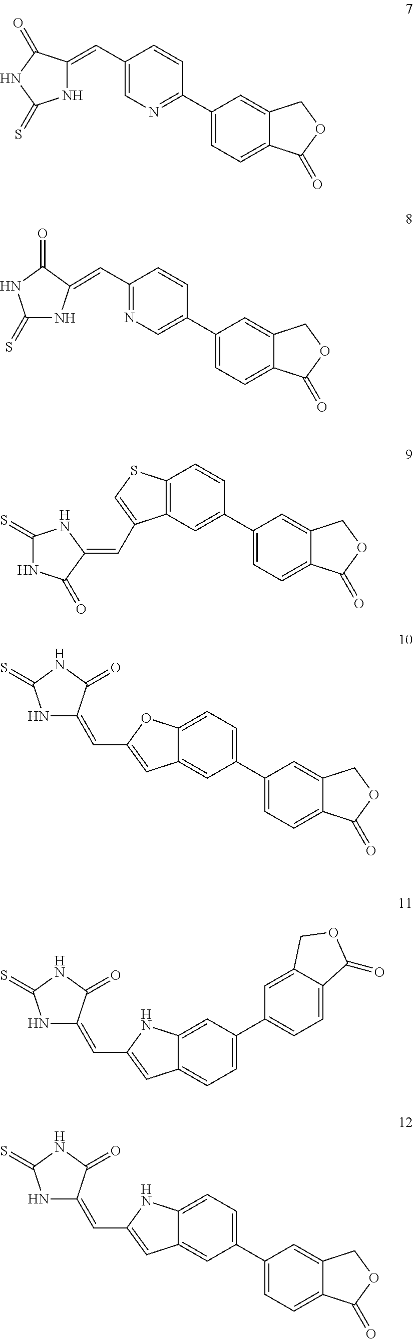 [SMILES: O=C1NC(=S)N/C1=C\c1ccc(-c2ccc3c(c2)COC3=O)nc1 ; O=C1NC(=S)N/C1=C\c1ccc(-c2ccc3c(c2)COC3=O)cn1 ; O=C1NC(=S)N/C1=C\c1csc2ccc(-c3ccc4c(c3)COC4=O)cc12 ; O=C1NC(=S)N/C1=C/c1cc2cc(-c3ccc4c(c3)COC4=O)ccc2o1 ; O=C1NC(=S)N/C1=C/c1cc2ccc(-c3ccc4c(c3)COC4=O)cc2[nH]1 ; O=C1NC(=S)N/C1=C/c1cc2cc(-c3ccc4c(c3)COC4=O)ccc2[nH]1]

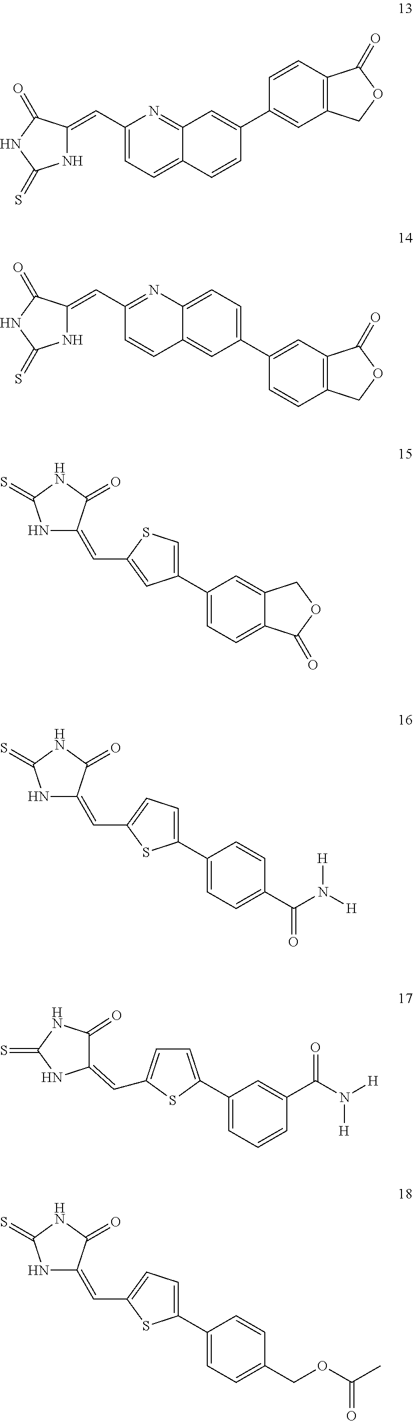 [SMILES: O=C1NC(=S)N/C1=C\c1ccc2ccc(-c3ccc4c(c3)COC4=O)cc2n1 ; O=C1NC(=S)N/C1=C\c1ccc2cc(-c3ccc4c(c3)C(=O)OC4)ccc2n1 ; O=C1NC(=S)N/C1=C/c1cc(-c2ccc3c(c2)COC3=O)cs1 ; NC(=O)c1ccc(-c2ccc(/C=C3/NC(=S)NC3=O)s2)cc1 ; NC(=O)c1cccc(-c2ccc(/C=C3/NC(=S)NC3=O)s2)c1 ; CC(=O)OCc1ccc(-c2ccc(/C=C3/NC(=S)NC3=O)s2)cc1]

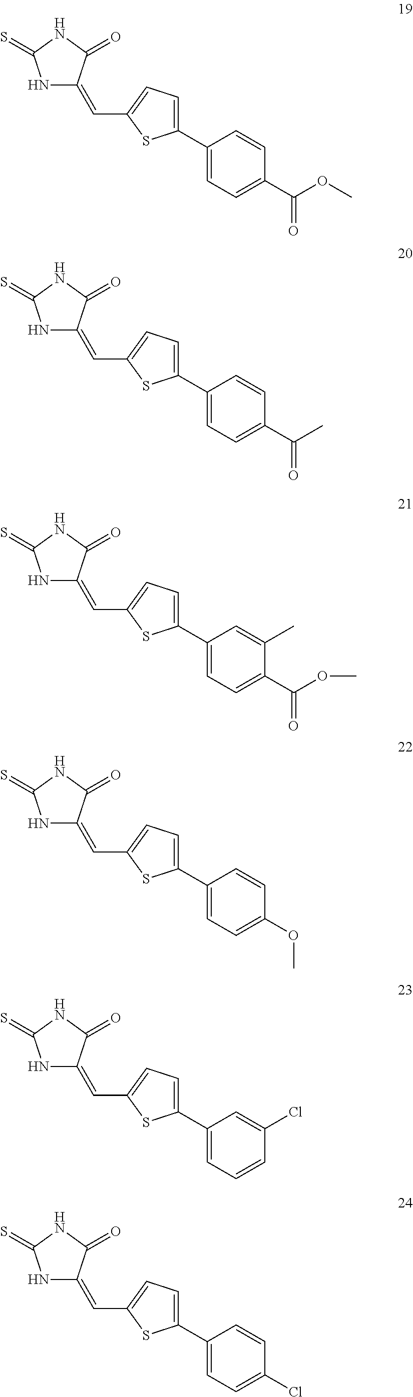 [SMILES: COC(=O)c1ccc(-c2ccc(/C=C3/NC(=S)NC3=O)s2)cc1 ; CC(=O)c1ccc(-c2ccc(/C=C3/NC(=S)NC3=O)s2)cc1 ; COC(=O)c1ccc(-c2ccc(/C=C3/NC(=S)NC3=O)s2)cc1C ; COc1ccc(-c2ccc(/C=C3/NC(=S)NC3=O)s2)cc1 ; O=C1NC(=S)N/C1=C/c1ccc(-c2cccc(Cl)c2)s1 ; O=C1NC(=S)N/C1=C/c1ccc(-c2ccc(Cl)cc2)s1]

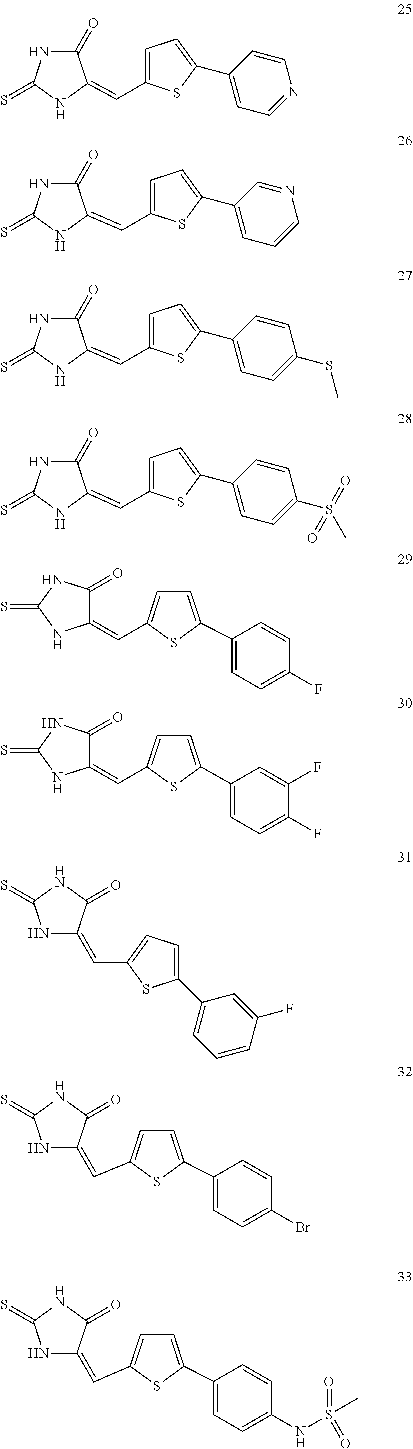

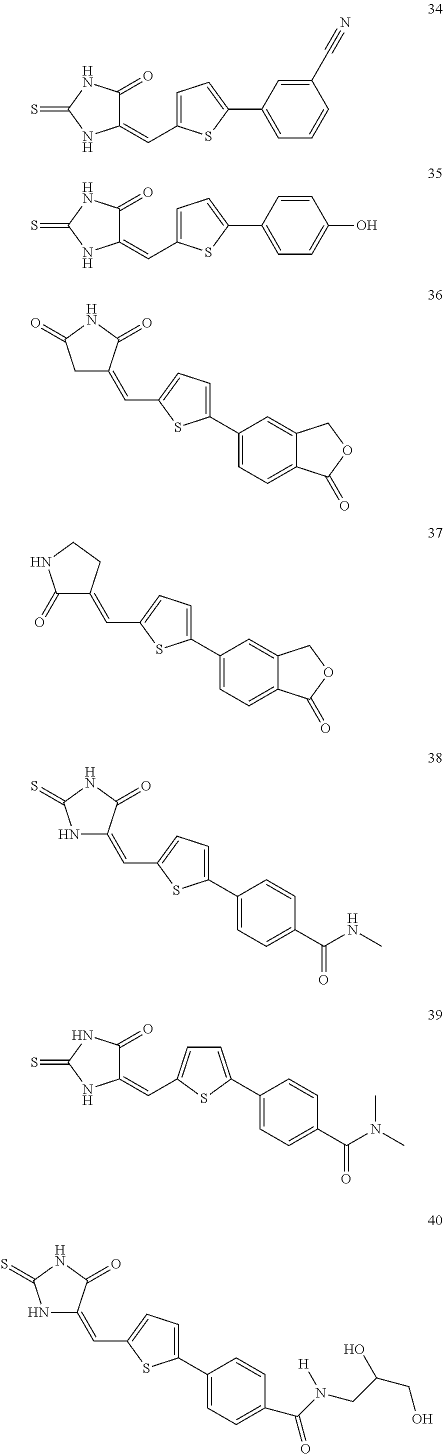

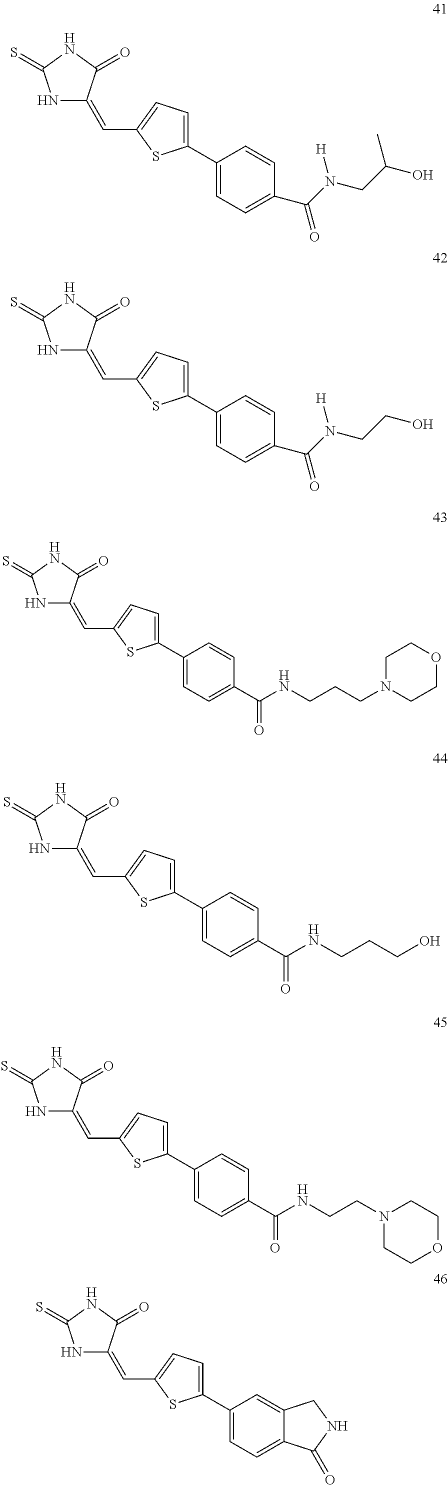 [SMILES: CC(O)CNC(=O)c1ccc(-c2ccc(/C=C3/NC(=S)NC3=O)s2)cc1 ; O=C1NC(=S)N/C1=C/c1ccc(-c2ccc(C(=O)NCCO)cc2)s1 ; O=C1NC(=S)N/C1=C/c1ccc(-c2ccc(C(=O)NCCCN3CCOCC3)cc2)s1 ; O=C1NC(=S)N/C1=C/c1ccc(-c2ccc(C(=O)NCCCO)cc2)s1 ; O=C1NC(=S)N/C1=C/c1ccc(-c2ccc(C(=O)NCCN3CCOCC3)cc2)s1 ; O=C1NC(=S)N/C1=C/c1ccc(-c2ccc3c(c2)CNC3=O)s1]

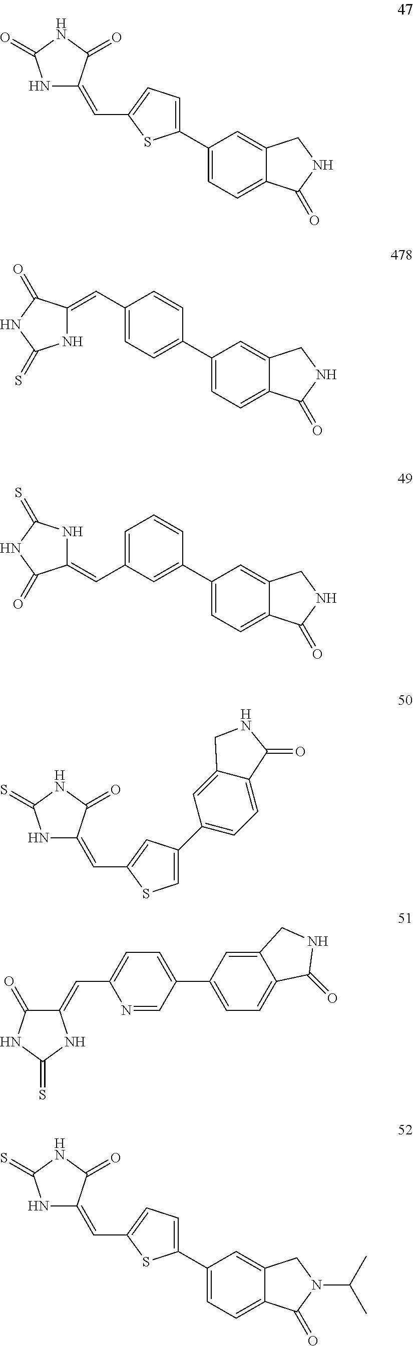 [SMILES: O=C1NC(=O)/C(=C\c2ccc(-c3ccc4c(c3)CNC4=O)s2)N1 ; O=C1NC(=S)N/C1=C\c1ccc(-c2ccc3c(c2)CNC3=O)cc1 ; O=C1NC(=S)N/C1=C\c1cccc(-c2ccc3c(c2)CNC3=O)c1 ; O=C1NC(=S)N/C1=C/c1cc(-c2ccc3c(c2)CNC3=O)cs1 ; O=C1NC(=S)N/C1=C\c1ccc(-c2ccc3c(c2)CNC3=O)cn1 ; CC(C)N1Cc2cc(-c3ccc(/C=C4/NC(=S)NC4=O)s3)ccc2C1=O]

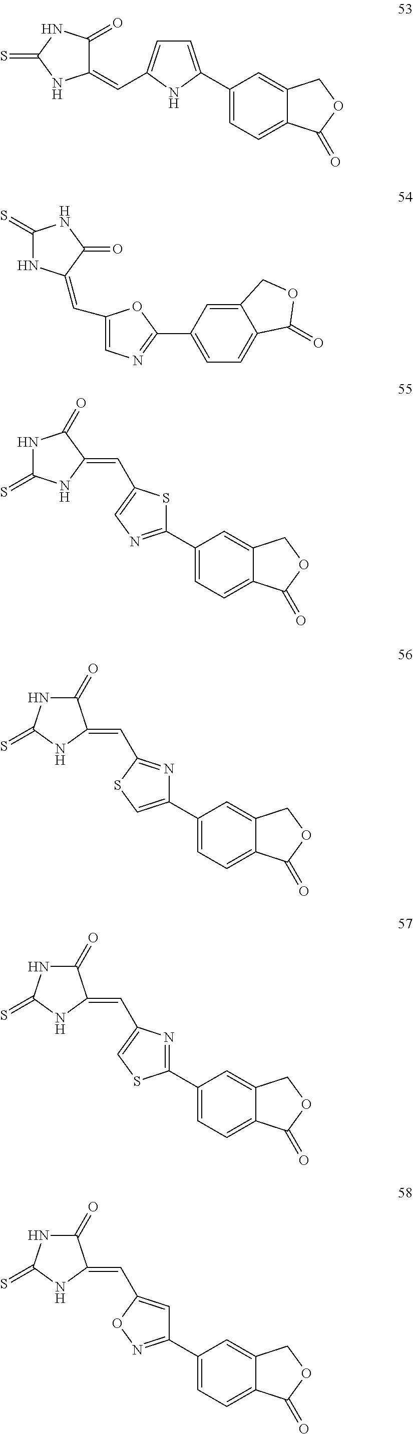 [SMILES: O=C1NC(=S)N/C1=C/c1ccc(-c2ccc3c(c2)COC3=O)[nH]1 ; O=C1NC(=S)N/C1=C/c1cnc(-c2ccc3c(c2)COC3=O)o1 ; O=C1NC(=S)N/C1=C\c1cnc(-c2ccc3c(c2)COC3=O)s1 ; O=C1NC(=S)N/C1=C\c1nc(-c2ccc3c(c2)COC3=O)cs1 ; O=C1NC(=S)N/C1=C\c1csc(-c2ccc3c(c2)COC3=O)n1 ; O=C1NC(=S)N/C1=C\c1cc(-c2ccc3c(c2)COC3=O)no1]

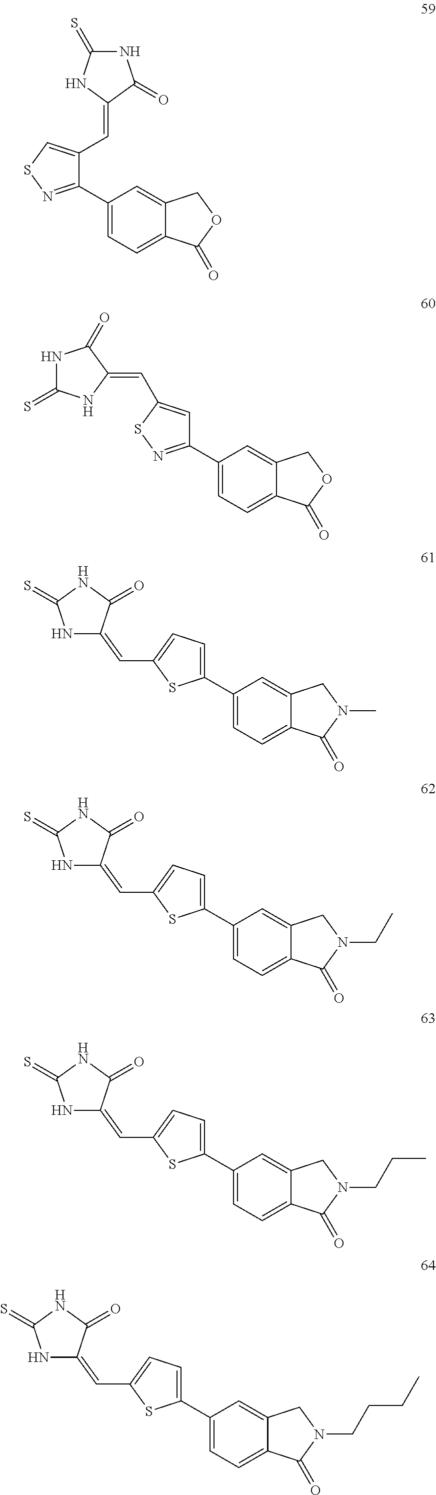 [SMILES: O=C1NC(=S)N/C1=C\c1csnc1-c1ccc2c(c1)COC2=O ; O=C1NC(=S)N/C1=C\c1cc(-c2ccc3c(c2)COC3=O)ns1 ; CN1Cc2cc(-c3ccc(/C=C4/NC(=S)NC4=O)s3)ccc2C1=O ; CCN1Cc2cc(-c3ccc(/C=C4/NC(=S)NC4=O)s3)ccc2C1=O ; CCCN1Cc2cc(-c3ccc(/C=C4/NC(=S)NC4=O)s3)ccc2C1=O ; CCCCN1Cc2cc(-c3ccc(/C=C4/NC(=S)NC4=O)s3)ccc2C1=O]

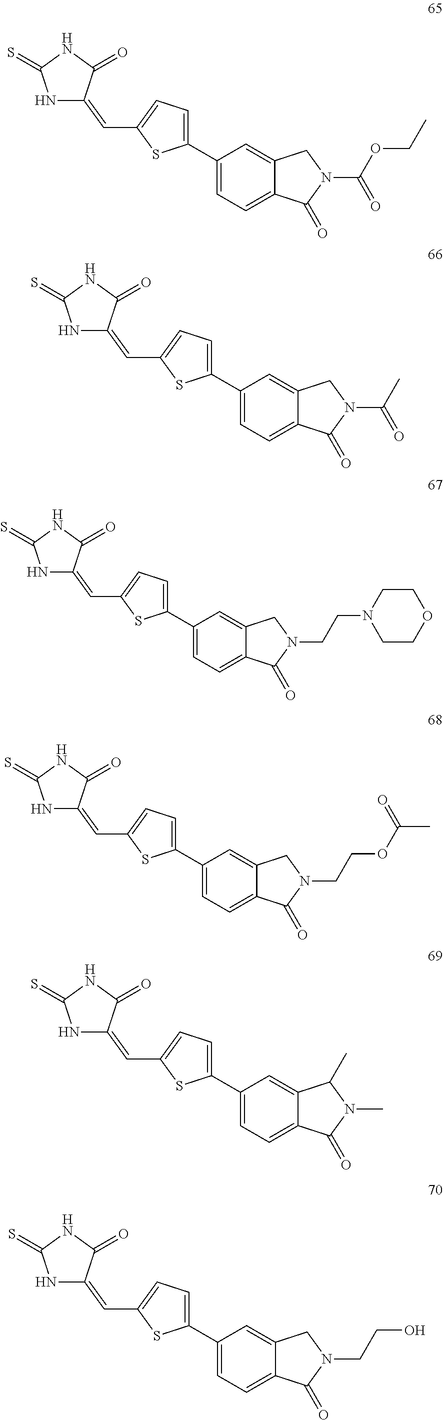 [SMILES: CCOC(=O)N1Cc2cc(-c3ccc(/C=C4/NC(=S)NC4=O)s3)ccc2C1=O ; CC(=O)N1Cc2cc(-c3ccc(/C=C4/NC(=S)NC4=O)s3)ccc2C1=O ; O=C1NC(=S)N/C1=C/c1ccc(-c2ccc3c(c2)CN(CCN2CCOCC2)C3=O)s1 ; CC(=O)OCCN1Cc2cc(-c3ccc(/C=C4/NC(=S)NC4=O)s3)ccc2C1=O ; CC1c2cc(-c3ccc(/C=C4/NC(=S)NC4=O)s3)ccc2C(=O)N1C ; O=C1NC(=S)N/C1=C/c1ccc(-c2ccc3c(c2)CN(CCO)C3=O)s1]

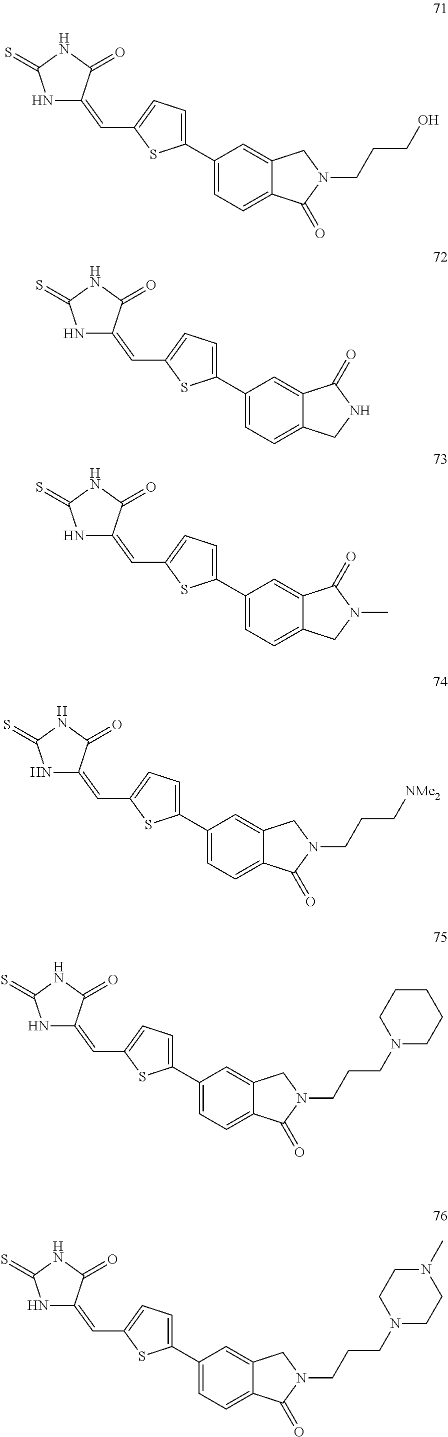 [SMILES: O=C1NC(=S)N/C1=C/c1ccc(-c2ccc3c(c2)CN(CCCO)C3=O)s1 ; O=C1NC(=S)N/C1=C/c1ccc(-c2ccc3c(c2)C(=O)NC3)s1 ; CN1Cc2ccc(-c3ccc(/C=C4/NC(=S)NC4=O)s3)cc2C1=O ; CN(C)CCCN1Cc2cc(-c3ccc(/C=C4/NC(=S)NC4=O)s3)ccc2C1=O ; O=C1NC(=S)N/C1=C/c1ccc(-c2ccc3c(c2)CN(CCCN2CCCCC2)C3=O)s1 ; CN1CCN(CCCN2Cc3cc(-c4ccc(/C=C5/NC(=S)NC5=O)s4)ccc3C2=O)CC1]

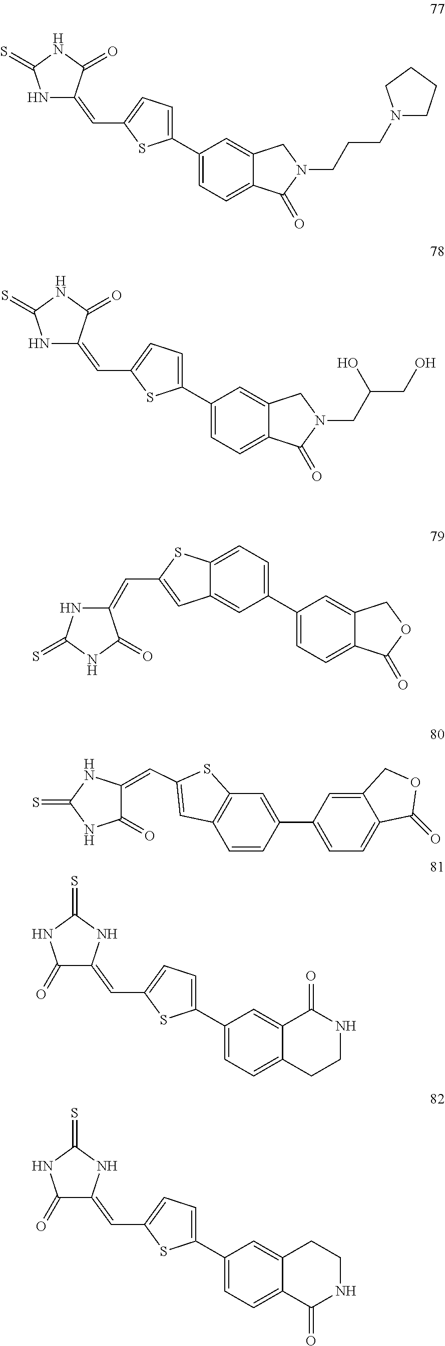 [SMILES: O=C1NC(=S)N/C1=C/c1ccc(-c2ccc3c(c2)CN(CCCN2CCCC2)C3=O)s1 ; O=C1NC(=S)N/C1=C/c1ccc(-c2ccc3c(c2)CN(CC(O)CO)C3=O)s1 ; O=C1NC(=S)N/C1=C/c1cc2cc(-c3ccc4c(c3)COC4=O)ccc2s1 ; O=C1NC(=S)N/C1=C/c1cc2ccc(-c3ccc4c(c3)COC4=O)cc2s1 ; O=C1NC(=S)N/C1=C\c1ccc(-c2ccc3c(c2)C(=O)NCC3)s1 ; O=C1NC(=S)N/C1=C\c1ccc(-c2ccc3c(c2)CCNC3=O)s1]

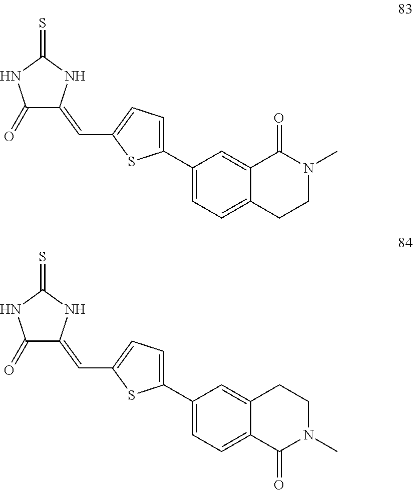 [SMILES: CN1CCc2ccc(-c3ccc(/C=C4\NC(=S)NC4=O)s3)cc2C1=O ; CN1CCc2cc(-c3ccc(/C=C4\NC(=S)NC4=O)s3)ccc2C1=O]

In another aspect of the present invention, there is provided a pharmaceutical composition including the compound of the present invention, as herein described and a pharmaceutically acceptable carrier, excipient, diluent and/or adjuvant.

The compounds of the present invention have been identified by the screening methods previously described in WO 2005/083098, the entire contents of which are incorporated herein by reference, and show an ability to inhibit the cytolytic activity of mouse, and human perforin. Given the degree of sequence homology of native perforin from different species, and the fact that the compounds identified by the present inventors are capable of inhibiting the cytolytic activity of human and mouse perforin, it is contemplated that the compounds of the present invention will also demonstrate an ability to inhibit the cytolytic activity of perforin from other species.

The terms “perforin”, “cytolysin”, “pore-forming protein (pfp)” and “C9-like protein” are used interchangeably herein and preferably encompass perforin polypeptides and fragments thereof in various forms, including naturally occurring or synthetic variants. Examples of perforins encompassed by the present invention include human perforin having an amino acid sequence as shown in FIG. 1. Also encompassed by the present invention are mouse and rat, perforin isoforms, although perforins derived from other species, including those that may be made by lower organisms such as bacteria, are also envisaged.

As used herein, the term “native perforin” preferably refers to a perforin polypeptide molecule having an amino acid sequence that occurs in nature (e.g., a natural protein). Native perforin, or naturally occurring perforin, may be identified as one of the main constituents of cytocidal granules, is found to migrate with a molecular mass of approximately 66 kDa upon reduction and SDS-polyacrylamide gel electrophoresis, and migrates more slowly under non-reducing conditions (70-75 kDa), suggestive of a tightly disulphide-bonded structure in its native form. In the presence of calcium ions (Ca²⁺), perforin monomers aggregate into tubular structures that span the lipid bilayer, producing circular lesions (varying between 6 and 20 nm in diameter) that are thought to grow in diameter through the progressive recruitment of additional monomers.

Variants of perforin may exhibit amino acid sequences that are at least 80% identical to a native perforin polypeptide or fragment thereof. Also contemplated are embodiments in which a variant comprises an amino acid sequence that is at least 90% identical, preferably at least 95% identical, more preferably at least 98% identical, even more preferably at least 99% identical, or most preferably at least 99.9% identical to the native perforin polypeptide or fragment thereof. Percent identity may be determined by visual inspection and mathematical calculation. Among the naturally occurring variants and fragments thereof provided are variants of native perforin that retain native biological activity or a substantial equivalent thereof. Also provided herein are naturally occurring variants that have enhanced biological activity as compared to a native perforin molecule.

Variants of perforin preferably include polypeptides that are substantially homologous to the native form of perforin, but which have an amino acid sequence different from that of the native form because of one or more deletions, insertions or substitutions. Preferred embodiments include polypeptides that comprise from one to ten deletions, insertions or substitutions of amino acid residues when compared to a native sequence. A given sequence may be replaced, for example, by a residue having similar physiochemical characteristics. Examples of conservative substitution of one aliphatic residue for another, such as Ile, Val, Leu or Ala for one another; substitution of one polar residue for another, such as between Lys and Arg, Glu and Asp, or Gln and Asn; or substitutions of one aromatic residue for another, such as Phe, Trp or Tyr for one another. Other conservative substitutions, e.g., involving substitutions of entire regions having similar hydrophobicity characteristics, are well known in the art. Variants may also be defined by the truncation of a native perforin polypeptide. Further variants encompassed by the present invention include, but are not limited to, deglycosylated perforin polypeptides, or fragments thereof, or those polypeptides demonstrating increased glycosylation when compared to native perforin. Also encompassed are perforin polypeptide variants with increased hydration. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue of a perforin polypeptide is preferably replaced with another amino acid residue from the same side chain family. Mutations may occur along all or part of a perforin coding sequence and the resultant mutants can be screened for perforin activity to identify variants that demonstrate the same or increased perforin activity in comparison to a native perforin molecule.

As used herein, the terms “perforin activity”, “biological activity of perforin” and the like preferably refer to the cytolytic activity of a perforin polypeptide; that is, its ability to bind to a target cell membrane and polymerise into pore-like transmembrane channels leading to cell lysis. The activity also includes the capacity to synergise with other toxins such as granule toxins and other molecules to induce apoptosis. The target cell can be any cell that is capable of being lysed by native perforin.

The biological activity of perforin can be assessed by the skilled addressee by any number of means known in the art including, but not limited to, the measurement of target cell lysis, the delivery of granzyme B molecules into the target cell, the measurement of target cell membrane disruption (such as by changes in ion transport), the induction of apoptosis in the target cell, the modification of vesicular trafficking and the general assessment of target cell death. The target cell may be a red blood cell (RBC) and hence a common means of measuring perforin activity is by a RBC lysis test. It may also be any nucleated cell.

As used herein, the term “fragment” preferably refers to a portion of a perforin polypeptide, or a variant thereof. Such fragments would retain biological activity as compared to a native perforin molecule.

In a further preferred embodiment, a fragment of a perforin polypeptide may consist of the biologically active C-terminal domain. Such fragments may generally be identified using techniques well known to those skilled in the art in identifying perforin activity, as herein described. Perforin polypeptide fragments may also be identified by screening fragments for their ability to react with perforin-specific antibodies and/or antisera. Antisera and antibodies are “perforin-specific” if they specifically bind to a perforin polypeptide or a variant or fragment thereof (i.e., they react with a perforin in an enzyme-linked immunosorbent assay [ELISA] or other immunoassay, and do not react detectably with unrelated polypeptides). Such antisera and antibodies may be prepared as described herein, and using well-known techniques (see, for example, Harlow and Lane, Antibodies: A Laboratory Manual, Cold Spring Harbor Laboratory, 1988).

It would be understood by one skilled in the art that the present invention is applicable to any species, including, but not limited to, human, rat, mouse, bird, horse, and lower organisms such as bacteria.

The compounds of the present invention have been identified by their ability to inhibit perforin activity, and as such, may be referred to herein as “inhibitors”, “perforin inhibitors”, “inhibitors of perforin activity”, and the like.

Preferably, the compounds of the present invention inhibit an activity of a perforin molecule, or a fragment or variant thereof, by binding the perforin molecule, or a fragment or variant thereof, and preventing the perforin molecule from contacting a target cell.

Alternatively, and without being limited by theory, the inhibitor may act in other ways including but not limited to preventing calcium binding by perforin molecules, preventing normal folding of perforin into an active configuration, preventing perforin from polymerising into a form capable of forming a transmembrane pore, or preventing perforin from effectively delivering other granule contents such as granzymes to induce apoptosis.

Alternatively, and without being limited by theory, the compounds of the present invention may inhibit the activity of a perforin molecule, or a fragment or variant thereof, by modulating a target cell, a receptor on the target cell or an interacting molecule such as a ligand on the surface of the target cell to which perforin is targeted such that the cell is modified to be less responsive to the perforin molecule.

The compounds of the present invention may be derived from natural sources, such as from bacterial, fungal, plant or animal extracts. Alternatively, numerous means are available to the skilled addressee for synthesis of the compound of the present invention.

### Pharmaceutically Acceptable Salts

The term “pharmaceutically acceptable” as applied to salts of the present invention and/or used in methods of the present invention refers to salts which are, within the scope of sound medical judgment, suitable for use in contact with the tissues of humans and lower animals without undue toxicity, irritation, allergic, or a like negative response that exceeds a reasonable risk/therapeutic benefit ratio. Preferably, a pharmaceutically acceptable salt is a salt that is suitable for administration to a patient. Accordingly, the present invention also extends to a pharmaceutically acceptable salt of any one of the compounds of the present invention.

Pharmaceutically acceptable salts are generally known in the art, and in the case of the present invention, include relatively non-toxic, organic or inorganic salts of the compounds of the present invention. Examples of such salts include, but are not limited to, acid addition salts such as hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naphthylate salts, mesylate salts, tosylate salts, citrate salts, lactate salts, maleate salts, succinate salts, tartrate salts, fumarate salts, and the like (see, for example. Berge et al., J. Pharm. Sci. 1977, 66:1-19). In addition, pharmaceutically acceptable salts also include basic salts such as alkali metal salts, alkaline earth salts, and ammonium salts. For example, pharmaceutically acceptable basic salts include salts of aluminum, calcium, lithium, magnesium, potassium, sodium, zinc, and the like. In addition, organic salts may also be used including, e.g., salts of lysine, N,N′-dibenzylethylenediamine, chloroprocaine, choline, diethanolamine, ethylenediamine, meglumine (N-methylglucamine), procaine and tris. The basic nitrogen-containing groups in the compounds of the present invention can be quaternized with various organic agents including, e.g., alkyl halides (such as lower alkyl halide including methyl, ethyl, propyl, and butyl chlorides, bromides and iodides), long chain halides (e.g., decyl, lauryl, myristyl and stearyl chlorides, bromides and iodides), aralkyl halides (e.g., benzyl and phenethyl bromides), dialkyl sulfates (e.g., dimethyl, diethyl, dibutyl and diamyl sulfates).

The pharmaceutically acceptable salts of the compounds of the present invention also can exist in the form of solvates, e.g., with water, methanol, ethanol, dimethylformamide, ethyl acetate, and the like, and mixtures thereof.

### Derivatives

The present invention also provides derivatives of the natural or synthetic compounds of the present invention through modification by conventional chemical, physical and biochemical means (see, e.g., Blondelle et al., 1996, Trends in Biotech. 14:60), or subjected to directed or random chemical modifications, such as acylation, alkylation, esterification, amidification, and the resultant analogs can be screened for their ability to modulate perforin activity, as herein described.

In a preferred embodiment, a derivative of the present invention is an ester, amide or hydrate of any one of the compounds of the present invention and/or used in methods of the invention. The term “pharmaceutically acceptable” preferably refers to esters, amides, or hydrates which are, within the scope of sound medical judgment, suitable for use in contact with the tissues of humans and lower animals without undue toxicity, irritation, allergic, or a like negative response that exceeds a reasonable risk/therapeutic benefit ratio.

Preferably, the pharmaceutically acceptable esters, amides, hydrates are esters, amides, hydrates suitable for administration to a patient.

Pharmaceutically acceptable esters can be made by reacting a hydroxyl group in the compounds of the present invention with a pharmaceutically acceptable organic acid, or by reacting a carboxylic acid group in the compounds with a pharmaceutically acceptable alcohol such as methanol, ethanol, propanol, etc. The organic acids used to form acid addition salts described above can all be useful.

Pharmaceutically acceptable amides can be prepared by reacting an amino functional group of the compounds of the above formulas with a pharmaceutically acceptable organic acid, as will be apparent to skilled artisans.

Derivatives of the present invention also encompass mimetics. Mimetics may be desirable where the active compound is difficult or expensive to synthesize or where it is unsuitable for a particular method of administration, e.g., peptides are generally unsuitable active agents for oral compositions as they tend to be quickly degraded by proteases in the alimentary canal. Mimetic design, synthesis, and testing are generally used to avoid large-scale screening of molecules for a target property.

When designing a mimetic, it is desirable to firstly determine the particular regions of the compound that are critical and/or important in determining the target property. In the case of a peptide, this can be done by systematically varying the amino acid residues in the peptide (e.g., by substituting each residue in turn). These parts or residues constituting the active region of the compound are known as its “pharmacophore”.

Once the pharmacophore has been found, its structure is modelled according to its physical properties (e.g., stereochemistry, bonding, size, and/or charge), using data from a range of sources (e.g. spectroscopic techniques, X-ray diffraction data, and NMR). Computational analysis, similarity mapping (which models the charge and/or volume of a pharmacophore, rather than the bonding between atoms), and other techniques can be used in this modelling process.

In a variant of this approach, the three dimensional structure of the compound and its binding partner are modelled. This can be especially useful where the compound and/or binding partner change conformation on binding, allowing the model to take account of this in the design of the mimetic.

A template molecule is then selected, and chemical groups that mimic the pharmacophore can be grafted onto the template. The template molecule and the chemical groups grafted on to it can conveniently be selected so that the mimetic is easy to synthesize, is likely to be pharmacologically acceptable, does not degrade in vivo, and retains the biological activity of the lead compound. The mimetics found are then screened to ascertain the extent they exhibit the target property, or to what extent they inhibit it. Further optimization or modification can then be carried out to arrive at one or more final mimetics for in vivo or clinical testing.

The compounds of the present invention may also be amended by adding one or more protected amino and/or hydroxyl groups by methods known to the skilled addressee. If the protective groups present are different, from one another, in many cases they can be removed selectively.

The term “amino protective group” is generally known to those skilled in the art and relates to groups which are suitable for protecting (for blocking) an amino group from chemical reactions, but which are easily removable after the desired chemical reaction has been carried out at other positions in the molecule. Typical groups of this type are, in particular, unsubstituted or substituted acyl, aryl, aralkoxymethyl or aralkyl groups. Since the protective groups are removed after the desired reaction (or reaction sequence), their nature and size is otherwise uncritical. Preferably, however, those having 1-20, in particular 1-8, C atoms are preferred. The expression “acyl group” is to be interpreted in the widest sense in connection with the present process. It includes acyl groups derived from aliphatic, araliphatic, aromatic or heterocyclic carboxylic acids or sulfonic acids and also, in particular, alkoxycarbonyl, aryloxycarbonyl and especially aralkoxycarbonyl groups. Examples of acyl groups of this type are alkanoyl such as acetyl, propionyl, butyryl; aralkanoyl such as phenylacetyl; aroyl such as benzoyl or toluoyl; aryloxy-alkanoyl such as POA; alkoxycarbonyl such as methoxycarbonyl, ethoxycarbonyl, 2,2,2-trichloroethoxycarbonyl, BOC, 2-iodoethoxycarbonyl; aralkyloxycarbonyl such as CBZ (“carbobenzoxy”), 4-methoxy-benzyloxycarbonyl, FMOC; arylsulfonyl such as Mtr Pbf or Pmc. Preferred amino protective groups are BOC and Mtr, additionally CBZ, Fmoc, benzyl and acetyl.

The term “hydroxyl protective group” is likewise generally known and relates to groups which are suitable for protecting a hydroxyl group from chemical reactions, but which are easily removable after the desired chemical reaction has been carried out at other positions in the molecule. Typical groups of this type are the abovementioned unsubstituted or substituted aryl, aralkyl or acyl groups and additionally also alkyl groups. The nature and size of the hydroxyl protective groups is not critical, since they are removed again after the desired chemical reaction or reaction, sequence; groups containing 1-20, in particular 1-10, C atoms are preferred. Examples of hydroxyl protective groups are, inter alia, benzyl, p-nitro-benzyl, p-toluenesulfonyl, tert-butyl and acetyl, benzyl and tert-butyl being particularly preferred. The COOH groups in aspartic acid and glutamic acid are preferably protected in the form of their tert-butyl esters (e.g. Asp(OtBu)).

The ability of the derivatives of the present invention to inhibit the cytolytic activity of perforin can be assessed by any number of means available to the skilled addressee, as taught, for example, in WO 2005/083098.

The present invention also provides compositions comprising a complex of a perforin inhibitor, or a pharmaceutically acceptable salt or a derivative thereof, as herein described, bound to a targeting molecule that is capable of enhancing delivery of the compound by providing for increased specificity, efficiency and duration of therapeutic action. Such targeting molecules comprise immunoconjugates, fusion proteins, and liposomes, microparticles, bioerodable polymers, gels, and foams. The targeting molecule may also comprise a targeting receptor molecule which enhancing the delivery of the perforin inhibitor to a cell or tissue, particularly to a cell or tissue which expresses a ligand to that receptor. The receptor may be derived from natural sources, or it may be synthesized by methods known in the art. In these compositions, the compounds of the present invention may remain substantially inactive or unavailable in the absence of a targeted receptor molecule to which they specifically bound.

The term “ligand” refers to a specific binding partner of a receptor and includes, without limitation, receptor agonists, partial agonists, mixed agonists, antagonists, drugs, hormones, transmitters, autocoids, growth factors, cytokines, prosthetic groups, coenzymes, cofactors, regulatory factors, antigens, haptens, vitamins, nucleic acids and synthetic heteropolymers comprising amino acids, nucleotides, carbohydrates or nonbiologic monomers, including analogs and derivatives thereof, and conjugates or complexes formed by attaching or binding any of these molecules to a second molecule. The term “receptor” refers to a specific binding partner of a ligand and includes, without limitation, membrane receptors, soluble receptors, cloned receptors, recombinant receptors, hormone receptors, drug receptors, transmitter receptors, autocoid receptors, cytokine receptors, antibodies, antibody fragments, engineered antibodies, antibody mimics, molecular recognition units, adhesion molecules, agglutinins, integrins, selectins, nucleic acids and synthetic heteropolymers comprising amino acids, nucleotides, carbohydrates or nonbiologic monomers, including analogs and derivatives thereof, and conjugates or complexes formed by attaching or binding any of these molecules to a second molecule.

“Prodrug derivatives” are also included in the scope of the present invention, wherein the perforin inhibitor, or a derivative thereof, is further modified with, for example, alkyl or acyl groups, sugars or oligopeptides, which are rapidly cleaved in the body to give the active compounds according to the invention. That is, the term “prodrug” refers to a precursor or modified compound of the present invention that is not fully active or available until converted in vivo to its therapeutically active or available form.

The term “synthetic receptor” refers to any naturally occurring, recombinant, biologically produced or synthetic ligand or receptor which is designed, selected or engineered to specifically bind a drug. The terms “therapeutic receptor” and “pathophysiologic receptor” refer to the molecular site of drug action. The term “therapeutic target” refers to an object of therapeutic intervention, including any physiologic or pathologic entity comprising therapeutic receptors, such as a specified organ, tissue or type of cell, platelet, corpuscle, microorganism, molecular complex or molecule.

### Pharmaceutical Compositions

In yet another aspect of the present invention, there is provided a pharmaceutical composition including a compound of the present invention, or a pharmaceutically acceptable salt or a derivative thereof, as herein described (also referred to herein as an “active compound”). In a preferred embodiment, the pharmaceutical composition may comprise a pharmaceutically acceptable carrier, excipient, diluent and/or adjuvant.

Pharmaceutical compositions of the present invention may be employed alone or in conjunction with other compounds, such as therapeutic compounds.

As used herein, the term “pharmaceutically acceptable carrier” preferably includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is generally formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, or liquid polyetheylene glycol, and the like), and suitable mixtures thereof. The proper fluidity can be maintained, for example, by the use of a coating such as lecithin, by the maintenance of the required particle size in the case of a dispersion or by the use of surfactants. Prevention of the action of microorganisms can be achieved by incorporation of various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, or sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required; followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yield a powder of the active ingredient plus any additional desired ingredient from a previously sterile-filtered solution thereof.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring.

For administration by inhalation, the compounds are delivered in the form of an aerosol spray from a pressurised container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished with nasal sprays or suppositories. The compounds can be prepared in the form of suppositories (e.g., with conventional suppository bases such as cocoa butter and other glycerides) or retention enemas for rectal delivery.

For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. “Dosage unit form” as used herein preferably refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosages for use in humans. The dosage lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate perforin activity is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell, R. J. et al. (1996) Current Opinion in Biotechnology 7:89-94 and in Shea, K. J. (1994) Trends in Polymer Science 2:166-173. Such “imprinted” affinity matrices are amenable to ligand-binding assays, whereby the immobilized monoclonal antibody component is replaced by an appropriately imprinted matrix. An example of the use of such matrices in this way can be seen in Vlatakis, G. et al. (1993) Nature 361:645-647. Through the use of isotope-labeling, the “free” concentration of compound which modulates the expression or activity of perforin can be readily monitored and used in calculations of IC₅₀. Such “imprinted” affinity matrices can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized, based on its individual IC₅₀. A rudimentary example of such a “biosensor” is discussed in Kriz, D. et al. (1995) Analytical Chemistry 67:2142-2144.

The skilled artisan will appreciate that certain factors may influence the dosage and timing required to effectively treat a subject, including the activity of the specific compound employed, the age, body weight, general health, gender, and diet of the subject, the time of administration, the route of administration, the rate of excretion, any drug combination, the degree of expression or activity to be modulated, the severity of the disease or disorder, previous treatments and other diseases present.

The pharmaceutical compositions according to the present invention can be included in a container, pack, or dispenser together with instructions for administration.

### Uses

It is another aspect of the present invention to provide a method of inhibiting activity of a perforin molecule, or a fragment or variant thereof, on a cell, said method comprising exposing the cell to a compound, or a pharmaceutically acceptable salt or a derivative thereof, as herein described. The cell may be a target cell (as herein described), or, alternatively, it may be a CTL and/or NK cell that express perforin. The exposing of the cell to the compound, or a pharmaceutically acceptable salt or a derivative thereof (as herein described), may occur in vitro, ex vivo or in vivo.

Where the exposing of a cell to the compound, occurs in vitro or ex vivo, for example, the method of the present invention may be used as a diagnostic tool to determine the efficacy of certain compounds (alone or in combination) for inhibiting perforin activity in a patient. For example, a CTL and/or NK cell that expresses perforin may be removed from a patient and exposed to one or more compounds of the present invention (or pharmaceutically acceptable salts or derivatives thereof) in the presence of a suitable target cell (as herein described). The target cell may, though need not be, from the same patient. In another example, a target cell may be removed from a patient and exposed to one or more compounds of the present invention (or pharmaceutically acceptable salts or derivatives thereof) in the presence of perforin. The ability of the compound (or compounds) to inhibit the activity of perforin can be assessed by measuring the degree of target cell lysis by any method known to one skilled in the art. Thus, one may be able to ascertain whether a certain compound is more efficacious than another and tailor a specific treatment regime to that patient.

In a preferred embodiment, the exposing of the cell to the compound, or a pharmaceutically acceptable salt or a derivative thereof, as herein described is in vivo.

### Methods of Treatment

In yet a further aspect of the present invention, there is provided a prophylactic or therapeutic method of treating a subject at risk of or susceptible to a disease or disorder, or having a disease or disorder, associated with aberrant perforin expression and/or activity. Such disease or disorder will generally be associated with either an increase in levels of perforin molecules, an increase in perforin activity as compared to a healthy population, or a pathological attack of the subject's tissues or by CTL, NK cells or other lymphocytes that utilise the perforin pathway.

In a preferred embodiment, the prophylactic or therapeutic method comprises the steps of administering a compound according to the present invention, or a pharmaceutically acceptable salt or a derivative thereof (as herein described), to a subject who has a disease or disorder, a symptom of disease or disorder, or predisposition toward a disease or disorder associated with undesired perforin activity as herein described, for the purpose to cure, heal alleviate, relieve, alter, remedy, ameliorate, improve, or affect the disease or disorder, the symptoms of the disease or disorder, or the predisposition towards the disease or disorder.

The prophylactic or therapeutic methods of the present invention may also comprise the administering of a combination of the compounds according to the present invention, or pharmaceutically acceptable salts or derivatives thereof (as herein described), to a subject who has a disease or disorder, a symptom of disease or disorder, or predisposition toward a disease or disorder associated with undesired perforin activity as herein described, for the purpose to cure, heal alleviate, relieve, alter, remedy, ameliorate, improve, or affect the disease or disorder, the symptoms of the disease or disorder, or the predisposition towards the disease or disorder. It is envisaged that certain combinations of compounds of the present invention (or pharmaceutically acceptable salts or derivatives thereof) may provide enhanced inhibition of perforin activity in comparison to prophylactic or therapeutic methods that utilise only one of the compounds of the present invention (or pharmaceutically acceptable salts or derivatives thereof).

It would also be appreciated by one skilled in the art that the prophylactic or therapeutic methods as herein described could be used in any number of combinations with other treatment modalities currently employed in the art.

With regard to both prophylactic and therapeutic methods of treatment, such treatments may be specifically tailored or modified, based on knowledge obtained from the field of pharmacogenomics. “Pharmacogenomics”, as used herein, preferably refers to the application of genomics technologies such as gene sequencing, statistical genetics, and gene expression analysis to drugs in clinical development and on the market. More preferably, the term refers to the study of how a patient's genes determine his or her response to a drug (e.g., a patient's “drug response phenotype”, or “drug response genotype”). Thus, another aspect of the present invention provides methods for tailoring an individual's prophylactic or therapeutic treatment with either the perforin molecules of the present invention or agents that modulate perforin expression and/or activity (such as those identified by screening assays as herein described), according to that individual's drug response genotype. Pharmacogenomics allows a clinician or physician to target prophylactic or therapeutic treatments to patients who will most benefit from the treatment and to avoid treatment of patients who will experience toxic drug-related side effects.

Conditions in which perforin expression and/or activity is increased, and where it is desirable to reduce said activity, may be identified by those skilled in the art by any or a combination of diagnostic or prognostic assays known in the art. For example, a biological sample obtained from a subject (e.g. blood, serum, plasma, urine, saliva, and/or cells derived therefrom) may be analysed for perforin expression and/or activity or the presence of CTL, NK cells or other lymphocytes capable of using perforin to induce tissue damage, as hereinbefore described. Such conditions include, but are not limited to, juvenile diabetes mellitus (type 1 or insulin dependent), graft-versus-host disease, chronic or acute allograft rejection, malaria and any other conditions associated with cytotoxic T lymphocyte- or natural killer cell-mediated immune pathology.

Thus, in one embodiment of the present invention, the prophylactic and therapeutic methods of treatment are applicable to the treatment and/or prevention of immune mediated conditions such as, but not limited to autoimmune diseases, such as juvenile diabetes mellitus (type 1 or insulin dependent), crohns disease, Guillain-Barre syndrome, lupus erythematosus, psoriasis, rheumatoid arthritis, vasculitis and Wegener's granulmatosis, as well as other conditions including but not limited to graft-versus-host disease, chronic or acute allograft rejection, infectious diseases, including mosquito-borne diseases of the Plasmodium genus, such as malaria, in particular cerebral malaria, and conditions associated with cytotoxic T lymphocyte- or natural killer cell-mediated immune pathology.

It is considered that the above methods are suitable for the prophylactic and therapeutic treatment of any species, including, but not limited to, all mammals including humans, cannines, felines, cattle, horses, rats and mice, as well as birds, reptiles and lower organisms such as bacteria.

For the above mentioned indications, the appropriate dosage will vary depending on, e.g. the compound employed, the age, sex, weight and general physical condition of the subject, the mode of administration, the nature and/or severity of the condition or the desired effect. By balancing these features it is well within the general skill of a medical practitioner to determine appropriate dosages. By way of example, however, suitable daily dosages are in the range of from about from about 0.1 to about 2000 mg/kg, preferably from about 0.2 to about 100 mg/kg, more preferably from about 0.5 to about 200 mg/kg, even more preferably from about 1 to about 50 mg/kg of body weight.

To assist in modifying those cells that may be targeted for lysis by perforin, the compounds employed in the prophylactic or therapeutics methods of the present invention may be attached to an identifying moiety such as an antibody so that the moiety identifies and targets the compound to those cells which require the modification of perforin activity. In conjunction with the treatment of diseases or disorders associated with undesired perforin expression and/or activity, pharmacogenomics (i.e., the study of the relationship between an individual's genotype and that individual's response to a foreign compound or drug) may also be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent to modulate perforin expression and/or activity, as well as tailoring the dosage and/or therapeutic regimen of such treatment.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms.

One pharmacogenomic approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug's target is known (i.e., perforin), all common variants of that gene can be fairly easily identified in the population and it can be determined if having one version of the gene versus another is associated with a particular drug response.

Alternatively, a method termed the “gene expression profiling” can be utilized to identify genes that predict drug response. For example, the gene expression of an animal dosed with a drug (e.g., a compound according to the present invention) can give an indication whether gene pathways related to toxicity have been turned on.

Information generated from more than one of the above pharmacogenomic approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a therapeutic agent as hereinbefore described.

Monitoring the influence of agents (e.g., drugs) on the activity of perforin can be applied in clinical trials. For example, the effectiveness of a compound of the present invention to inhibit perforin activity or the lytic or pro-apoptotic activity of CTL or NK cells can be monitored in clinical trials of subjects exhibiting enhanced perforin, CTL or NK cell activity as compared to a healthy population. In such clinical trials, the activity of perforin, and preferably, other genes that have been implicated in, for example, conditions associated with undesired perforin expression and/or activity (i.e. surrogate markers) can be used as a “read out” or markers of the phenotype of a particular cell.

Examples of the procedures used in the present invention will now be more fully described. It should be understood, however, that the following description is illustrative only and should not be taken in any way as a restriction on the generality of the invention described above.

### EXAMPLES

### Example 1

### A. Methods for Preparing Compounds of Formula (I) of the Invention

The following examples are representative of the present invention, and provide detailed methods for preparing exemplary compounds of the present invention.

NMR spectra were obtained on a Bruker Avarice-400 spectrometer at 400 MHz for ¹H and 100 MHz for ¹³C spectra, referenced to Me₄Si. Low resolution mass spectra were obtained on a Thermo Finnigan Surveyor MSQ. High resolution mass spectra were recorded on a Varian VG 7070 spectrometer at nominal 5000 resolution. Analyses were performed by the Microchemical Laboratory, University of Otago, Dunedin, NZ. Melting points were determined using an Electrothermal Model 9200 or Gallenkamp digital melting point apparatus, and are as read. Column chromatography was carried out on silica gel, (Merck 230-400 mesh) unless otherwise stated.

### Example 2

### General Procedure A

### 5-(5-(1,3-Dioxolan-2-yl)furan-2-yl)isobenzofuran-1(3H)-one (85) (Scheme 1)

5-Bromo-2-furaldehyde was protected as the cyclic acetal according to a literature procedure¹. This cyclic acetal (666 mg, 3.04 mmol) was dissolved in toluene (27 mL), to which was added a suspension of 5-(4,4,5,5-tetramethyl-1,3,2-dioxaborolan-2-yl)isobenzofuran-1(3H)-one (527 mg, 2.03 mmol) in EtOH (10 mL). This boronate ester was prepared in turn from 5-bromoisobenzofuran-1(3H)-one² according to a literature procedure.³ The entire mixture was heated at reflux under nitrogen for 2 h., then upon cooling, all solvents were removed under reduced pressure and the resulting residue was partitioned between water (50 mL) and CH₂Cl₂ (50 mL). Two further CH₂Cl₂ (50 mL) extractions were performed, then the combined organic fractions dried (Na₂SO₄), filtered, and the solvent removed under reduced pressure to afford a residue which was purified by flash column chromatography on silica gel (10% EtOAc/hexanes as eluant). The title compound was isolated as a beige solid (416 mg, 75%). ¹H NMR [400 MHz, (CD₃)₂SO] δ 7.98 (s, 1H), 7.92 (dd, J=8.1, 1.2 Hz, 1H), 7.88 (d, J=8.1 Hz, 1H), 7.21 (d, J=3.5 Hz, 1H), 6.73 (d, J=3.4 Hz, 1H), 5.98 (s, 1H), 5.44 (s, 2H), 4.05-4.14 (m, 2H), 3.94-4.03 (m, 2H).

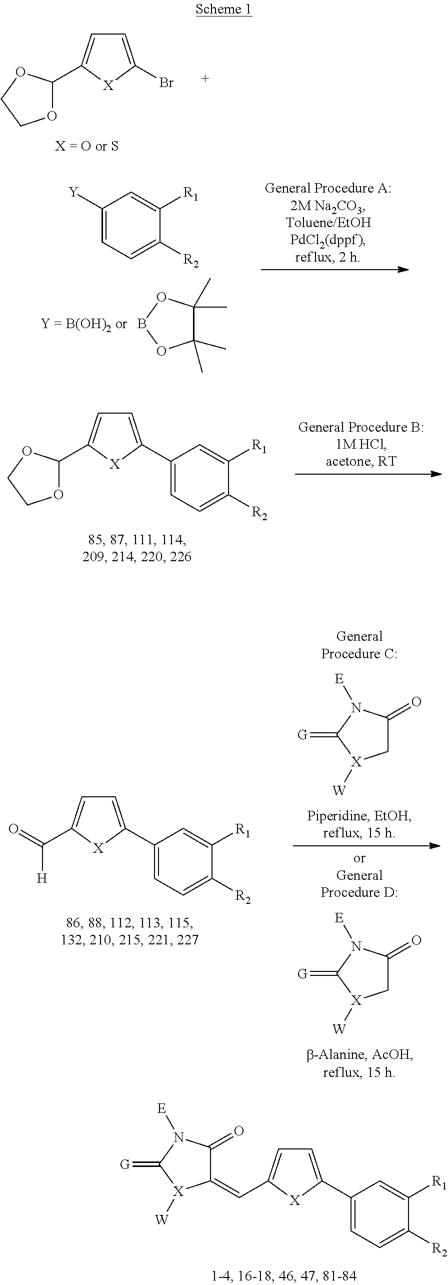

### Example 3

### General Procedure B

### 5-(1-Oxo-1,3-dihydroisobenzofuran-5-yl)furan-2-carbaldehyde (86) (Scheme 1)

5-(5-(1,3-Dioxolan-2-yl)furan-2-yl)isobenzofuran-1(3H)-one (416 mg, 1.53 mmol) was dissolved in acetone (20 mL), to which was added 1 M HCl (4 mL). The resulting solution was stirred at RT for 3 h., at which point an off-white precipitate had crashed out of solution. The mixture was diluted with water (100 mL) and extracted with CH₂Cl₂ (4×100 mL). The combined CH₂Cl₂ fractions were dried (Na₂SO₄), filtered, and the solvent removed under reduced pressure to give a solid which was triturated with Et₂O to afford the title compound as a beige solid upon filtration (292 mg, 84%), mp (Et₂O) 229-231° C. ¹H NMR [400 MHz, (CD₃)₂SO] δ 9.68 (s, 1H), 8.17 (br s, 1H), 8.09 (dt, J=8.0, 0.7 Hz, 1H), 7.96 (d, J=8.0 Hz, 1H), 7.71 (d, J=3.8 Hz, 1H), 7.53 (d, J=3.8 Hz, 1H), 5.48 (s, 2H). Anal. (C₁₃H₈O₄) H, N, C; +0.5.

### Example 4

### General Procedure C

### (E,Z)-5-((5-(1-Oxo-1,3-dihydroisobenzofuran-5-yl)furan-2-yl)methylene)-2-thioxoimidazolidin-4-one (1) (Scheme 1)

5-(1-Oxo-1,3-dihydroisobenzofuran-5-yl)furan-2-carbaldehyde (50 mg, 0.22 mmol) was suspended in EtOH (10 mL), to which was added 2-thiohydantoin (26 mg, 0.23 mmol) and piperidine (1 drop). This mixture was stirred at RT for 72 h. The title compound was collected by filtration, directly from the reaction mixture, as a brown solid (57 mg, 80%), mp (EtOH)>300° C. ¹H NMR [400 MHz, (CD₃)₂SO] δ 12.20 (br s, 1H), 12.02 (br s, 1H), 8.18 (br s, 1 H), 8.15 (d, J=8.2 Hz, 1H), 7.92 (d, J=8.1 Hz, 1H), 7.44 (d, J=3.8 Hz, 1H), 7.38 (d, J=3.8 Hz, 1H), 6.44 (s, 1H), 5.49 (s, 2H). LRMS (APCI⁺) calcd for C₁₆H₁₁N₂O₄S 327 (MH⁺), found 327. Anal. (C₁₆H₁₀N₂O₄S.0.75H₂O) C, H, N.

### Example 5

### 5-(5-(1,3-Dioxolan-2-yl)thiophen-2-yl)isobenzofuran-1(3H)-one (87)

5-Bromo-2-thiophenecarboxaldehyde was protected as the cyclic acetal according to a literature procedure'. This compound was then reacted with 5-(4,4,5,5-tetramethyl-1,3,2-dioxaborolan-2-yl)isobenzofuran-1(3H)-one according to general procedure A.

Purification of the resulting product by flash column chromatography on silica gel (CH₂Cl₂ as eluant) gave the title compound as a cream solid (56%). ¹H NMR [400 MHz, (CD₃)₂SO] δ 7.95 (br s, 1H), 7.84-7.90 (m, 2H), 7.63 (d, J=3.8 Hz, 1H), 7.29 (d, J=3.7 Hz, 1H), 6.08 (s, 1H), 5.43 (s, 2H), 4.01-4.11 (m, 2H), 3.92-4.00 (m, 2H). LRMS (APCI⁺) calcd for C₁₅H₁₃O₄S 289 (MH⁺), found 289.

### Example 6

### 5-(1-Oxo-1,3-dihydroisobenzofuran-5-yl)thiophene-2-carbaldehyde (88)

5-(5-(1,3-Dioxolan-2-yl)thiophen-2-yl)isobenzofuran-1(3H)-one was deprotected according to general procedure B. Trituration with Et₂O gave the title compound as a biege solid (89%), mp (Et₂O) 224-226° C. ¹H NMR [400 MHz, (CD₃)₂SO] δ 9.96 (s, 1H), 8.12 (br s, 1H), 8.11 (d, J=4.0 Hz, 1H), 8.02 (dd, J=8.1, 1.5 Hz, 1H), 7.93 (d, J=7.9 Hz, 1H), 7.33 (d, J=4.0 Hz, 1H), 5.47 (s, 2H). Anal. (C₁₃H₈O₃) H. C; +0.5.

### Example 7

### General Procedure D

### (E,Z)-5-((5-(1-Oxo-1,3-dihydroisobenzofuran-5-yl)thiophen-2-yl)methylene)-2-thioxoimidazolidin-4-one (2) (Scheme 1)

5-(1-Oxo-1,3-dihydroisobenzofuran-5-yl)thiophene-2-carbaldehyde (50 mg, 0.21 mmol), 2-thiohydantoin (26 mg, 0.23 mmol) and β-alanine (20 mg, 0.23 mmol) were suspended in AcOH (5 mL) and the mixture heated at reflux for 15 h. Upon cooling, a yellow-brown solid crystallized out of solution and was collected by filtration. This material did not require further purification. The title compound was isolated as a orange-brown solid after drying under vacuum (52 mg, 78%), mp (AcOH)>300° C. ¹H NMR [400 MHz, (CD₃)₂SO] δ 12.40 (br s, 1H), 12.02 (br s, 1H), 7.99 (br s, 1H), 7.93 (dd, J=8.1, 1.4 Hz, 1H), 7.83-7.91 (m, 3H), 6.65 (s, 1H), 5.45 (s, 2H). Anal. (C₁₆H₁₀N₂O₃S₂) H, N. C; +0.5.

### Example 8

### (E,Z)-5-((5-(1-Oxo-1,3-dihydroisobenzofuran-5-yl)thiophen-2-yl)methylene)imidazolidine-2,4-dione (3)

The title compound was prepared by reaction of 5-(1-oxo-1,3-dihydroisobenzofuran-5-yl)thiophene-2-carbaldehyde with hydantoin according to general procedure C. Trituration with THF afforded the desired product as a yellow solid (44%), mp (EtOH)>300° C. ¹H NMR [400 MHz, (CD₃)₂SO] δ 10.20-11.50 (v br s, 2H), 7.96 (br s, 1H), 7.86-7.92 (m, 2H), 7.81 (d, J=4.0 Hz, 1H), 7.65 (d, J=4.0 Hz, 1H), 6.60 (s, 1H), 5.49 (s, 2H). Anal. (C₁₆H₁₀N₂O₄S) C, H, N.

### Example 9

### (E,Z)-2-Imino-1-methyl-5-((5-(1-oxo-1,3-dihydroisobenzofuran-5-yl)thiophen-2-yl)methylene)imidazolidin-4-one (4)

Reaction of 5-(1-oxo-1,3-dihydroisobenzofuran-5-yl)thiophene-2-carbaldehyde with creatinine according to general procedure D gave the title compound. Trituration with dioxane gave the desired product as a yellow-orange solid (95%), mp (AcOH)>300° C. ¹H NMR [400 MHz, (CD₃)₂SO] δ 11.91 (v br s, 1H), 7.94 (br s, 1H), 7.96 (br s, 1H), 7.84-7.91 (m, 2H), 7.67 (d, J=3.9 Hz, 1H), 7.63 (d, J=4.2 Hz, 1H), 6.61 (s, 1H), 5.44 (s, 2H), 3.21 (s, 3H). HRMS (FAB⁺) calcd for C₁₇H₁₄N₃O₃S 340.0756 (MH⁺), found 340.07550. Anal. (C₁₇H₁₃N₃O₃S.0.75H₂O) C, H, N.

### Example 10

### 5-(3-(Dimethoxymethyl)phenyl)isobenzofuran-1(3H)-one (89) (Scheme 2)

3-Bromobenzaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a white solid (59%). ¹H NMR [400 MHz, (CD₃)₂SO] δ 7.97 (d, J=0.5 Hz, 1H), 7.93 (d, J=8.0 Hz, 1H); 7.87 (dd, J=8.0, 1.4 Hz, 1H), 7.72-7.77 (m, 2H), 7.52-7.58 (m, 1H), 7.47 (d, J=7.7 Hz, 1H), 5.48 (s, 1H), 5.47 (s, 2H), 3.30 (s, 6H). LRMS (APCI⁺) calcd for C₁₇H₁₇O₄ 285 (MH⁺), found 285.

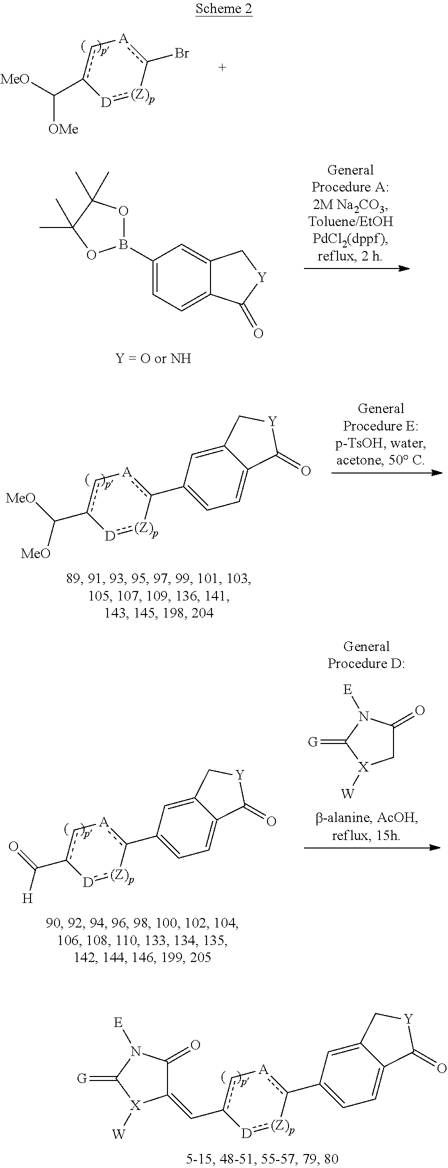

### Example 11

### General Procedure E

### 3-(1-Oxo-1,3-dihydroisobenzofuran-5-yl)benzaldehyde (90) (Scheme 2)

5-(3-(Dimethoxymethyl)phenyl)isobenzofuran-1(3H)-one (97 mg, 0.34 mmol) was dissolved in a mixture of acetone (6 mL) and water (2 mL), then toluenesulfonic acid (20 mg) added, and the mixture stirred at 50° C. for 5 h. After cooling, the reaction mixture was partitioned between sat. NaHCO₃ solution (50 mL) and CH₂Cl₂ (50 mL). An additional extraction was carried out with CH₂Cl₂ (50 mL), then the combined organic extracts dried (Na₂SO₄), filtered, and the solvent removed under reduced pressure to afford the aldehyde which did not require further purification. The desired product was isolated as a cream solid (86%). ¹NMR [400 MHz, (CD₃)₂SO] δ 10.13 (s, 1 H), 8.30 (t, J=1.6 Hz, 1H), 8.12 (dq, J=7.7, 1.9, 1.1 Hz, 1H), 8.06 (t, J=0.9 Hz, 1H), 8.00 (dt, J=7.7, 1.3 Hz, 1H), 7.97 (d, J=1.1 Hz, 2H), 7.77 (t, J=7.7 Hz, 1H), 5.49 (s, 2 H). LRMS (APCI⁺) calcd for C₁₃H₁₁O₃ 239 (MH⁺), found 239.

### Example 12

### (E,Z)-5-(3-(1-Oxo-1,3-dihydroisobenzofuran-5-yl)benzylidene)-2-thioxoimidazolidin-4-one (5) (Scheme 2)

Reaction of 3-(1-oxo-1,3-dihydroisobenzofuran-5-yl)benzaldehyde with 2-thiohydantoin according to general procedure D gave the title compound. Trituration with Et₂O gave the desired product as a yellow solid (65%), mp (AcOH) 285-288° C. ¹H NMR [400 MHz, (CD₃)₂SO] δ 12.33 (br s, 2H), 8.04 (br s, 2H), 7.92-8.00 (m, 2H), 7.81 (br d, J=7.8 Hz, 1H), 7.74 (br d, J=8.0 Hz, 1H), 7.57 (t, J=7.8 Hz, 1H), 6.58 (s, 1H), 5.50 (s, 2H). LRMS (APCI⁺) calcd for C₁₈H₁₁N₂O₃S 335 (M-H), found 335. Anal. (C₁₈H₁₂N₂O₃S.0.5H₂O) C, H, N.

### Example 13

### 5-(4-(Dimethoxymethyl)phenyl)isobenzofuran-1(3H)-one (91)

4-Bromobenzaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a white solid (51%). ¹H NMR [400 MHz, (CD₃)₂SO] δ 7.97 (d, J=0.4 Hz, 1H), 7.93 (dd, J=8.0, 0.5 Hz, 1H), 7.88 (dd, J=8.0, 1.4 Hz, 1H), 7.78 (d, J=8.4 Hz, 2H), 7.53 (d, J=8.2 Hz, 2H), 5.47 (s, 2H), 5.46 (s, 1H), 3.30 (s, 6H). LRMS (APCI⁺) calcd for C₁₇H₁₇O₄ 285 (MH⁺), found 285.

### Example 14

### 4-(1-Oxo-1,3-dihydroisobenzofuran-5-yl)benzaldehyde (92)

5-(4-(Dimethoxymethyl)phenyl)isobenzofuran-1(3H)-one was deprotected according to general procedure E to give the title compound as a white solid (87%). ¹H NMR [400 MHz, (CD₃)₂SO] δ 10.10 (s, 1H), 8.03-8.08 (m, 3H), 7.95-8.02 (m, 4H), 5.49 (s, 2H). LRMS (APCI⁺) calcd for C₁₅H₁₁O₃ 239 (MH⁺), found 239.

### Example 15

### (E,Z)-5-(4-(1-Oxo-1,3-dihydroisobenzofuran-5-yl)benzylidene)-2-thioxoimidazolidin-4-one (6)

Reaction of 4-(1-oxo-1,3-dihydroisobenzofuran-5-yl)benzaldehyde with 2-thiohydantoin according to general procedure D gave the title compound. The resulting solid was isolated by filtration from the reaction mixture and did not require further purification. The desired product was obtained as a yellow solid (89%), mp (AcOH)>300° C. ¹H NMR [400 MHz, (CD₃)₂SO] δ 12.29 (s, 2H), 8.03 (br s, 1H), 7.88-7.97 (m, 4 H), 7.83 (d, J=8.5 Hz, 2H), 6.54 (s, 1H), 5.48 (s, 2H). LAMS (APCI⁻) calcd for C₁₈H₁₁N₂O₃S 335 (M-H), found 335. Anal. (C₁₈H₁₂N₂O₃S) C, H, N.

### Example 16

### 5-(6-(Dimethoxymethyl)pyridin-3-yl)isobenzofuran-1(3H)-one (93)

2-Bromopyridine-5-carboxaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (CH₂Cl₂, followed by 10% acetone/CH₂Cl₂ as eluant) gave the desired product as a white solid (34%). ¹H NMR [400 MHz, (CD₃)₂SO] δ 8.79 (s, 1H), 8.19 (s, 1H), 8.13 (d, J=8.1 Hz, 1H), 8.01 (d, J=8.0 Hz, 1H), 7.91 (dd, J=8.2, 2.0 Hz, 1H), 7.82 (d, J=8.2 Hz, 1H), 5.52 (s, 1H), 5.45 (s, 2H), 3.38 (s, 6H). LRMS (APCI⁺) calcd for C₁₆H₁₆NO₄ 286 (MH⁺), found 286.

### Example 17

### 5-(1-Oxo-1,3-dihydroisobenzofuran-5-yl)picolinaldehyde (94)

5(6-(Dimethoxymethyl)pyridin-3-yl)isobenzofuran-1(3H)-one was deprotected according to general method E to give the title compound as a white solid (41%). ¹H NMR [400 MHz, (CD₃)₂SO] δ 10.18 (s, 1H), 9.22 (dd, J=2.1, 0.8 Hz, 1H), 8.48 (br s, 1H), 8.37-8.42 (m, 2H), 8.33 (d, J=8.2 Hz, 1H), 8.00 (d, J=8.2 Hz, 1H), 5.52 (s, 2H). LRMS (APCI') calcd for C₁₄H₁₀NO₃ 240 (MH⁺), found 240.

### Example 18

### ((E,Z)-5-((5-(1-oxo-1,3-dihydroisobenzofuran-5-yl)pyridin-2-yl)methylene)-2-thioxoimidazolidin-4-one (7)

Reaction of 5-(1-oxo-1,3-dihydroisobenzofuran-5-yl)picolinaldehyde with 2-thiohydantoin according to general procedure D gave the title compound as a yellow solid (89%), mp (AcOH)>300° C. ¹H NMR [400 MHz, (CD₃)₂SO] δ 12.38 (br s, 2H), 9.00 (d, J=2.2 Hz, 1H), 8.45 (s, 1H), 8.38 (d, J=8.1 Hz, 2H), 8.17 (d, J=8.4 Hz, 1H), 7.97 (d, J=8.1 Hz, 1H), 6.57 (s, 1H), 5.50 (s, 2H). LRMS (APCI⁺) calcd for C₁₇H₁₀N₃O₃S 336 (M-H), found 336. Anal. (C₁₇H₁₁N₃O₃S) C, H, N.

### Example 19

### 5-(6-(Dimethoxymethyl)pyridin-3-yl)isobenzofuran-1(3H)-one (95)

5-Bromopyridine-2-carboxaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (CH₂Cl₂, followed by 10% acetone/CH₂Cl₂ as eluant) gave the desired product as an off-white solid (68%). ¹H NMR [400 MHz, CDCl_{3]} δ 8.87 (d, j=1.9 Hz, 1H), 8.03 (d, J=8.0 Hz, 1H), 7.97 (dd, J=8.1, 2.4 Hz, 1H), 7.74 (d, J=8.1 Hz, 1H), 7.66-7.72 (m, 2H), 5.45 (s, 1H), 5.40 (s, 2H), 3.45 (s, 6H). LRMS (APCI⁺) calcd for C₁₆H₁₆NO₄ 286 (MH⁺), found 286.

### Example 20

### 5-(1-Oxo-1,3-dihydroisobenzofuran-5-yl)picolinaldehyde (96)

5-(6-(Dimethoxymethyl)pyridin-3-yl)isobenzofuran-1(3H)-one was deprotected according to general method E to give the title compound as an off-white solid (88%). ¹H NMR [400 MHz, (CD₃)₂SO] δ 10.06 (s, 1H), 9.23 (dd, J=2.3, 0.7 Hz, 1H), 8.44 (ddd, J=8.1, 2.3, 0.7 Hz, 1H), 8.16 (d, J=0.7 Hz, 1H), 7.98-8.09 (m, 3H), 5.51 (s, 2H). LRMS (APCI⁺) calcd for C₁₄H₁₀NO₃ 240 (MH⁺), found 240.

### Example 21

### (E,Z)-5-((5-(1-oxo-1,3-dihydroisobenzofuran-5-yl)pyridin-2-yl)methylene)-2-thioxoimidazolidin-4-one (8)

Reaction of 5-(1-oxo-1,3-dihydroisobenzofuran-5-yl)picolinaldehyde with 2-thiohydantoin according to general procedure D, followed by trituration with Et₂O, gave the title compound as a yellow-green solid (80%), mp (AcOH)>300° C. ¹H NMR [400 MHz, (CD₃)₂SO] δ 12.54 (br s, 1H), 11.52 (br s, 1H), 9.15 (d, J=2.4 Hz, 1H), 8.29 (dd, J=8.2, 2.5 Hz, 1H), 8.09 (s, 1H), 7.96-8.02 (m, 2H), 7.87 (d, J=8.2 Hz, 1H), 6.69 (s, 1H), 5.50 (s, 2H). LRMS (APCI⁻) calcd for C₁₇H₁₀N₃O₃S 336 (M-H), found 336. Anal. (C₁₇H₁₁N₃O₃S) C, H, N.

### Example 22

### 5-(3-(Dimethoxymethyl)benzo[b]thiophen-5-yl)isobenzofuran-1(3H)-one (97)

5-Bromobenzothiophene-3-carboxaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a white solid (56%). ¹H NMR [400 MHz, (CD₃)₂SO] δ 8.25 (d, J=1.5 Hz, 1H), 8.16 (d, J=8.5 Hz, 1H), 8.01 (d, J=0.4 Hz, 1H), 7.96 (d, J=8.1 Hz, 1H), 7.92 (dd, J=8.1, 1.4 Hz, 1H), 7.84 (s, 1H), 7.77 (dd, J=8.4, 1.8 Hz, 1H), 5.84 (s, 1H), 5.50 (s, 2H), 3.30 (s, 6H). LRMS (APCI⁺) calcd for C₁₉H₁₇O₄S 341 (MH⁺), found 341.

### Example 23

### 5-(1-Oxo-1,3-dihydroisobenzofuran-5-yl)benzo[b]thiophene-3-carbaldehyde (98)

5-(3-(Dimethoxymethyl)benzo[b]thiophen-5-yl)isobenzofuran-1(3H)-one was deprotected according to general method E to give the title compound as a white solid (100%). ¹H NMR [400 MHz, (CD₃)₂SO] δ 10.19 (s, 1H), 9.07 (s, 1H), 8.88 (d, J=1.5 Hz, 1H), 8.28 (d, J=8.0 Hz, 1H), 8.04 (br s, 1H), 7.92-8.00 (m, 2H), 7.89 (dd, J=8.5, 1.9 Hz, 1H), 5.51 (s, 2H). LRMS (APCI⁺) calcd for C₁₇H₁₁O₃S 295 (MH⁺), found 295.

### Example 24

### (E,Z)-5-4(5-(1-Oxo-1,3-dihydroisobenzofuran-5-yl)benzo[b]thiophen-3-yl)methylene)-2-thioxoimidazolidin-4-one (9)

Reaction of 5-(1-oxo-1,3-dihydroisobenzofuran-5-yl)benzo[b]thiophene-3-carbaldehyde with 2-thiohydantoin according to general procedure D gave the title compound. The resulting solid was isolated by filtration from the reaction mixture, and did not require further purification. The desired product was obtained as a yellow solid (97%), mp (AcOH)>300° C. ¹H NMR [400 MHz, (CD₃)₂SO] δ 12.28 (br s, 2 H), 8.56 (s, 1H), 8.41 (d, J=1.5 Hz, 1H), 8.22 (d, J=8.5 Hz, 1H), 8.16 (d, J=0.7 Hz, 1H), 8.07 (dd, J=8.0, 1.4 Hz, 1H), 7.94 (d, J=8.0 Hz, 1H), 7.86 (dd, J=8.5, 1.7 Hz, 1H), 6.95 (s, 1H), 5.50 (s, 2H). Anal. (C₂₀H₁₂N₂O₃S₂.0.5H₂O) C, H, N.

### Example 25

### 5-(2-(Dimethoxymethyl)benzofuran-5-yl)isobenzofuran-1(3H)-one (99)

5-Bromobenzofuran-2-carbaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a white solid (53%). ¹H NMR [400 MHz; CDCl₃] δ 7.99 (d, J=8.0 Hz, 1H), 7.81 (d, J=1.8 Hz, 1H), 7.77 (dd, J=8.0, 1.4 Hz, 1H), 7.68 (d, J=0.6 Hz, 1H), 7.60 (d, J=8.6 Hz, 1H), 7.54 (d, J=8.6, 1.9 Hz, 1H), 6.76 (t, J=0.8 Hz, 1H), 5.60 (d, J=0.8 Hz, 1H), 5.38 (s, 2H), 3.45 (s, 6H). LRMS (APCI⁺) calcd for C₁₉H₁₇O₅ 325 (MH⁺), found 325.

### Example 26

### 5-(1-Oxo-1,3-dihydroisobenzofuran-5-yl)benzofuran-2-carbaldehyde (100)

5-(2-(Dimethoxymethyl)benzofuran-5-yl)isobenzofuran-1(3H)-one was deprotected according to general method E to give the title compound as a white solid (80%). ¹H NMR [400 MHz, CDCl₃] δ9.93 (s, 1H), 7.98-8.03 (m, 2H), 7.69-7.79 (m, 4H), 7.63 (d, J=0.8 Hz, 1H), 5.40 (s, 2H). LRMS (APCI⁺) calcd for C₁₇H₁₁O₄ 279 (MH⁺), found 279.

### Example 27

### (2)-5-((5-(1-Oxo-1,3-dihydroisobenzofuran-5-yl)benzofuran-2-yl)methylene)-2-thioxoimidazolidin-4-one (10)

Reaction of 5-(1-oxo-1,3-dihydroisobenzofuran-5-yl)benzofuran-2-carbaldehyde with 2-thiohydantoin according to general procedure D gave the title compound. The resulting solid was isolated by filtration from the reaction mixture and did not require further purification. The desired product was obtained as a bright yellow solid (87%), mp (AcOH)>300° C. ¹H NMR [400 MHz, (CD₃)₂SO] δ 12.23 (br s, 2H), 8.06 (d, J=0.9 Hz, 1H), 8.00 (s, 1H), 7.93 (d, J=0.8 Hz, 2H), 7.77-7.82 (m, 2H), 7.50 (s, 1H), 6.56 (s, 1H), 5.48 (s, 2H). LRMS (APCI⁺) calcd for C₂₀H₁₃N₂O₄S 377 (MH⁺), found 377. Anal. (C₂₀H₁₂N₂O₄S0.10AcOH) C, H, N.

### Example 28

### 5-(2-(Dimethoxymethyl)-1H-indol-6-yl)isobenzofuran-1(3H)-one (101)

6-Bromo-1H-indole-2-carbaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a pale yellow solid (29%). ¹H NMR [400 MHz, CDCl₃] δ 8.53 (br s, 1H), 7.97 (d, J=8.1 Hz, 1H), 7.79 (d, J=7.9 Hz, 1H), 7.68-7.73 (m, 2H), 7.61 (t, J=0.8 Hz, 1H), 7.39 (dd, J=8.2, 1.6 Hz, 1H), 6.58 (t, J=1.0 Hz, 1H), 5.67 (d, J=0.5 Hz, 1H), 5.37 (s, 2H), 3.41 (s, 6H). LRMS (APCI⁺) calcd for C₁₉H₁₈NO₄ 324 (MH⁺), found 324.

### Example 29

### 6-(1-Oxo-1,3-dihydroisobenzofuran-5-yl)-1H-indole-2-carbaldehyde (102)

5-(2-(Dimethoxymethyl)-1H-indol-6-yl)isobenzofuran-1(3H)-one was deprotected according to general method E to give the title compound as a pale yellow solid (69%). ¹H NMR [400 MHz, CDCl₃] δ 9.89 (s, 1H), 9.12 (br s, 1H), 8.01 (d, J=8.0 Hz, 1H), 7.87 (d, J=8.4 Hz, 1H), 7.81 (d, J=7.5 Hz, 1H), 7.73 (s, 1H), 7.67 (s, 1H), 7.46 (dd, J=8.4, 1.5 Hz, 1H), 7.32 (q, J=0.9 Hz, 1H), 5.39 (s, 2H). LRMS (APCI⁺) calcd for C₁₇H₁₂NO₃ 278 (MH⁺), found 278.

### Example 30

### (E,Z)-5-((6-(1-Oxo-1,3-dihydroisobenzofuran-5-yl)-1H-indol-2-yl)methylene)-2-thioxoimidazolidin-4-one (11)

Reaction of 6-(1-oxo-1,3-dihydroisobenzofuran-5-yl)-1H-indole-2-carbaldehyde with 2-thiohydantoin according to general procedure D gave the title compound. The resulting solid was isolated by filtration from the reaction mixture and did not require further purification. The desired product was obtained as a bright red solid (74%), mp (AcOH)>300° C. ¹H NMR [400 MHz, (CD₃)₂SO] Observe E- and Z- isomers separately. δ 12.10-12.79 (br m, 2H), 12.03 (s, 0.8H), 11.591 (s, 0.2H), 7.90-8.02 (m, 4 H), 7.74 (s, 0.2H), 7.73 (d, J=8.4 Hz, 0.8H), 7.48 (dd, J=8.4, 1.6 Hz, 0.8H), 7.47 (dd, J=8.3, 1.7 Hz, 0.2H), 7.38 (s, 0.2H), 7.16 (s, 0.8H), 6.72 (s, 0.8H), 6.59 (s, 0.2H), 5.47 (s, 2H). LRMS (APCI⁺) calcd for C₂₀H₁₄N₃O₃S 376 (MH⁺), found 376. Anal. (C₂₀H₁₃N₃O₃S.0.25AcOH) C, H, N.

### Example 31

### 5-(2-(Dimethoxymethyl)-1H-indol-5-yl)isobenzofuran-1(3H)-one (103)

5-Bromo-1H-indole-2-carbaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired, compound as a pink solid (67%). ¹H NMR [400 MHz, CDCl₃] δ 8.50 (br s, 1H), 7.96 (d, J=8.0 Hz, 1H), 7.86 (s, 1H), 7.80 (d, J=8.0 Hz, 1H), 7.71 (s, 1H), 7.47 (s, 2H), 6.61 (d, J=1.8 Hz, 1H), 5.67 (s, 1H), 5.37 (s, 2H), 3.41 (s, 6H). LRMS (APCI⁺) calcd for C₁₉H₁₈NO₄ 324 (MH⁺), found 324.

### Example 32

### 5-(1-Oxo-1,3-dihydroisobenzofuran-5-yl)-1H-indole-2-carbaldehyde (104)

5-(2-(Dimethoxymethyl)-1H-indol-5-yl)isobenzofuran-1(3H)-one was deprotected according to general method E to give the title compound as a pale yellow solid (94%). ¹H NMR [400 MHz, CDCl₃] δ 12.12 (br s, 1H), 9.90 (s, 1H), 8.17 (d, J=1.2 Hz, 1H), 7.98 (s, 1H), 7.92 (s, 2H), 7.76 (dd, J=8.7, 1.8 Hz, 1H), 7.60 (d, J=8.7 Hz, 1H), 7.49 (d, J=0.8 Hz, 1H), 5.47 (s, 2H). LRMS (APCI⁺) calcd for C₁₇H₁₂NO₃ 278 (MH⁺), found 278.

### Example 33

### (E,Z)-5-((5-(1-Oxo-1,3-dihydroisobenzofuran-5-yl)-1H-indol-2-yl)methylene)-2-thioxoimidazolidin-4-one (12)

Reaction of 5-(1-oxo-1,3-dihydroisobenzofuran-5-yl)-1H-indole-2-carbaldehyde with 2-thiohydantoin according to general procedure D gave the title compound. The resulting solid was isolated by filtration from the reaction mixture and did not require further purification. The desired product was obtained as an orange-brown solid (78%), mp (AcOH)>300° C. ¹H NMR [400 MHz, (CD₃)₂SO] Observe E- and Z-isomers separately. δ 12.09-12.74 (br m, 2H), 12.03 (s, 0.8H), 11.59 (s, 0.2H), 7.90-8.03 (m, 4H), 7.74 (s, 0.2H), 7.72 (d, J=8.4 Hz, 0.8H), 7.48 (dd, J=8.4, 1.6 Hz, 0.8H), 7.46 (dd, J=8.3, 1.6 Hz, 0.2H), 7.37 (s, 0.2H), 7.16 (s, 0.8H), 6.72 (s, 0.8H), 6.59 (s, 0.2H), 5.47 (s, 2H). LRMS (APCI⁺) calcd for C₂₀H₁₄N₃O₃S 376 (MH⁺), found 376. Anal. (C₂₀H₁₃N₃O₃S.0.20AcOH) C, H, N.

### Example 34

### 5-(2-(Dimethoxymethyl)quinolin-7-yl)isobenzofuran-1(3H)-one (105)

7-Bromoquinoline-2-carbaldehyde was protected as the dimethyl acetal according to a literature procedure⁵ [except using HCl (g) as catalyst instead of p-TsOH], then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a yellow solid (62%). ¹H NMR [400 MHz, CDCl₃] δ 8.44 (t, J=0.9 Hz, 1H), 8.26 (d, J=8.3 Hz, 1H), 8.05 (d, J=7.9 Hz, 1H), 7.96 (d, J=8.5 Hz, 1H), 7.91 (dt, J=8.0, 0.8 Hz, 1H), 7.82-7.85 (m, 2H), 7.73 (d, J=8.5 Hz, 1H), 5.52 (s, 1H), 5.41 (s, 2H), 3.50 (s, 6H). LRMS (APCI⁺) calcd for C₂₀H₁₈NO₄ 336 (MH⁺), found 336.

### Example 35

### 7-(1-Oxo-1,3-dihydroisobenzofuran-5-yl)quinoline-2-carbaldehyde (106)

5-(2-(Dimethoxymethyl)quinolin-7-yl)isobenzofuran-1(3H)-one was deprotected according to general method B to give the title compound as a pale yellow solid (61%). ¹H NMR [400 MHz, CDCl₃] δ 10.25 (s, 1H), 8.52 (t, J=0.9 Hz, 1H), 8.38 (d, J=8.4 Hz, 1H), 8.09 (d, J=8.4 Hz, 1H), 8.08 (d, J=8.0 Hz, 1H), 8.05 (d, J=8.5 Hz, 1H), 7.97 (dd, J=8.5, 1.8 Hz, 1H), 7.94 (d, J=8.6 Hz, 1H), 7.87 (s, 1H), 5.43 (s, 2H). LRMS (APCI⁺) calcd for C₁₈H₁₂NO₃ 290 (MH⁺), found 290.

### Example 36

### (E,Z)-5-((6-(1-Oxo-1,3-dihydroisobenzofuran-5-yl)quinolin-2-yl)methylene)-2-thioxoimidazolidin-4-one (13)

Reaction of 7-(1-oxo-1,3-dihydroisobenzofuran-5-yl)quinoline-2-carbaldehyde with 2-thiohydantoin according to general procedure D gave the title compound. The resulting solid was isolated by filtration from the reaction mixture and did not require further purification. The desired product was obtained as a yellow-green solid (85%), mp (AcOH)>310° C. ¹H NMR [400 MHz, (CD₃)₂SO] δ 11.81 (br s, 2H), 8.77 (s, 1H), 8.47 (d, J=8.4 Hz, 1H), 8.20 (s, 1H), 8.12 (dd, J=8.6, 2.7 Hz, 2H), 8.03 (dd, J=8.5, 2.9 Hz, 2H), 7.87 (d, J=8.4 Hz, 1H), 6.74 (s, 1H), 5.54 (s, 2H). LRMS (APCI⁺) calcd for C₂₁H₁₄N₃O₃S 388 (MH⁺), found 388. Anal. (C₂₁H₁₃N₃O₃S.0.30AcOH) C, H, N.

### Example 37

### 5-(2-(Dimethoxymethyl)quinolin-6-yl)isobenzofuran-1(3H)-one (107)

6-Bromoquinoline-2-carbaldehyde was protected as the dimethyl acetal according to a literature procedure⁵ [except using HCl (g) as catalyst instead of p-TsOH], then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired compound as a pale yellow solid (84%). ¹H NMR [400 MHz, CDCl₃] δ 8.28 (dd, J=8.6, 1.6 Hz, 2H), 8.07 (d, J=2.0 Hz, 1H), 8.05 (d, J=8.0 Hz, 1H), 7.99 (dd, J=8.8, 2.1 Hz, 1H), 7.88 (d, J=8.0 Hz, 1H), 7.80 (s, 1H), 7.74 (d, J=8.5 Hz, 1H), 5.52 (s, 1H), 5.41 (s, 2H), 3.50 (s, 6H). LRMS (APCI⁺) calcd for C₁₂H₁₃BrNO₂ 283 (MH⁺), found 283.

### Example 38

### 6-(1-Oxo-1,3-dihydroisobenzofuran-5-yl)quinoline-2-carbaldehyde (108)

5-(2-(Dimethoxymethyl)quinolin-6-yl)isobenzofuran-1(3H)-one was deprotected according to general method B to give the title compound as a pale yellow solid (78%). ¹H NMR [400 MHz, CDCl₃] δ 10.26 (s, 1H), 8.40 (d, J=8.4 Hz, 1H), 8.39 (d, J=8.7 Hz, 1H), 8.04-8.14 (m, 4H), 7.91 (dt, J=8.0, 0.6 Hz, 1H), 7.83 (s, 1H), 5.43 (s, 2H). LRMS (APCI⁺) calcd for C₂₀H₁₈NO₄ 336 (MH⁺), found 336.

### Example 39

### (E,Z)-5-((6-(1-Oxo-1,3-dihydroisobenzofuran-5-yl)quinolin-2-yl)methylene)-2-thioxoimidazolidin-4-one (14)

Reaction of 6-(1-oxo-1,3-dihydroisobenzofuran-5-yl)quinoline-2-carbaldehyde with 2-thiohydantoin according to general procedure D gave the title compound. The resulting solid was isolated by filtration from the reaction mixture and did not require further purification. The desired product was obtained as a yellow-green solid (82%), mp (AcOH)>310° C. ¹H NMR [400 MHz, (CD₃)₂SO] δ 12.50 (br s, 1H), 11.86 (br s, 1H), 8.48 (t, J=8.2 Hz, 2H), 8.41 (d, J=2.1 Hz, 1H), 8.23 (dd, J=8.9, 2.1 Hz, 1H), 8.16 (s, 1H), 8.09 (dd; J=8.1, 1.3 Hz, 1H), 7.99 (d, J=8.0 Hz, 1H), 7.88 (d, J=8.5 Hz, 1H), 6.74 (s, 1H), 5.51 (s, 2H). LRMS (APCI⁺) calcd for C₂₁H₁₄N₃O₃S 388 (MH⁺), found 388. Anal. (C₂₁H₁₃N₃O₃S) C, H, N.

### Example 40

### 5-(5-(Dimethoxymethyl)thiophen-3-yl)isobenzofuran-1(3H)-one (109)

4-Bromo-2-thiophenecarboxaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired product as a white solid (44%). ¹H NMR [400 MHz, (CD₃)₂SO] δ 8.11 (d, J=1.6 Hz, 1H), 8.01 (d, J=0.6 Hz, 1H), 7.94 (dd, J=8.0, 1.4 Hz, 1H), 7.85 (d, J=8.0 Hz, 1H), 7.62 (dd, J=1.5, 0.8 Hz, 1H), 5.69 (s, 1H), 5.43 (s, 2H), 3.28 (s, 6H). LRMS (APCI⁺) calcd for C₁₅H₁₅O₄S 291 (MH⁺), found 291.

### Example 41

### 4-(1-Oxo-1,3-dihydroisobenzofuran-5-yl)thiophene-2-carbaldehyde (110)

5-(5-(Dimethoxymethyl)thiophen-3-yl)isobenzofuran-1(3H)-one was deprotected according to general method E to give the title compound as a white solid (87%). ¹H NMR [400 MHz, (CD₃)₂SO] δ 10.00 (d, J=1.3 Hz, 1H), 8.67 (t, J=1.4 Hz, 1H), 8.59 (d, J=2.0 Hz, 1H), 8.08 (d, J=0.6 Hz, 1H), 8.01 (dd, J=8.1, 1.4 Hz, 1H), 7.93 (d, J=7.9 Hz, 1H), 5.48 (s, 2H).

### Example 42

### (E,Z)-5-((4-(1-Oxo-1,3-dihydroisobenzofuran-5-yl)thiophen-2-yl)methylene)-2-thioxoimidazolidin-4-one (15)

Reaction of 4-(1-oxo-1,3-dihydroisobenzofuran-5-yl)thiophene-2-carbaldehyde with 2-thiohydantoin according to general procedure D gave the title compound. Trituration with dioxane gave the desired product as a yellow solid (80%), mp (AcOH)>300° C. ¹H NMR [400 MHz, (CD₃)₂SO] δ 12.39 (s, 1H), 12.21 (s, 1 H), 8.30-8.35 (m, 2H), 8.07 (br d, J=8.3 Hz, 2H), 7.94 (d, J=8.1 Hz, 1H), 6.56 (s, 1H), 5.49 (s, 2H). LRMS (APCI⁺) calcd for C₁₆H₉N₂O₃S₂ 341 (M-H), found 341. Anal. (C₁₆H₁₀N₂O₃S₂.0.25H₂O) C, H, N.

### Example 43

### 4-(5-(1,3-Dioxolan-2-yl)thiophen-2-yl)benzamide (111) (Scheme 1)

5-Bromo-2-thiophenecarboxaldehyde was protected as the cyclic acetal according to a literature procedure¹. This compound and 4-aminocarbonylphenylboronic acid were reacted according to general procedure A to give the title compound. Purification was carried out by flash column chromatography on silica gel (10% acetone/CH₂Cl₂ as eluant) to give the desired product as an off-white solid (64%). ¹H NMR [400 MHz, (CD₃)₂SO] δ 7.97 (br s, 1H), 7.91 (d, J=8.5 Hz, 2H), 7.72 (d, J=8.5 Hz, 2H), 7.52 (d, J=3.7 Hz, 1H), 7.34 (br s, 1H), 7.24 (d, J=3.7 Hz, 1H), 6.05 (s, 1H), 4.02-4.10 (m, 2H), 3.92-4.00 (m, 2H). LRMS (APCI⁺) calcd for C₁₄H₁₄NO₃S 276 (MH⁺), found 276.

### Example 44

### 4-(5-Formylthiophen-2-yl)benzamide (112)

4-(5-(1,3-Dioxolan-2-yl)thiophen-2-yl)benzamide was deprotected according to general procedure B. The title compound was isolated as a white solid (90%). ¹H NMR [400 MHz, (CD₃)₂SO] δ 9.94 (s, 1H), 8.07 (d, J=4.0 Hz, 1H), 8.05 (br s, 1H), 7.97 (d, J=8.6 Hz, 2H), 7.89 (d, J=8.6 Hz, 2H), 7.85 (d, J=4.0 Hz, 1H), 7.43 (br s, 1H). LRMS (APCI⁻) calcd for C₁₂H₉NO₂S 231 (M), found 231.

### Example 45

### (E,Z)-4-(5-((5-oxo-2-thioxoimidazolidin-4-ylidene)methyl)thiophen-2-yl)benzamide (16)

Reaction of 4-(5-formylthiophen-2-yl)benzamide with 2-thiohydantoin according to general procedure D gave the title compound. The resulting solid was isolated by filtration from the reaction mixture and did not require further purification. The desired product was obtained as an orange solid (92%), mp (AcOH)>300° C. ¹H NMR [400 MHz, (CD₃)₂SO] δ 12.38 (br s, 1H), 11.97 (br s, 1H), 8.00 (br s, 1H), 7.94 (d, J=8.5 Hz, 2H), 7.85 (br s, 1 H), 7.79 (d, J=8.5 Hz, 2H), 7.75 (d, J=4.0 Hz, 1H), 7.37 (br s, 1H), 6.64 (s, 1H). LAMS (APCI⁺) calcd for C₁₅H₁₂N₃O₂S₂ 330 (MH⁺), found 330. Anal. (C₁₅H₁₁N₃O₂S₂) C, H, N.

### Example 46

### 3-(5-Formylthiophen-2-yl)benzamide (113)

5-Bromo-2-thiophenecarboxaldehyde was protected as the cyclic acetal according to a literature procedure. This compound and 3-aminocarbonylphenylboronic acid were reacted according to general procedure A to give the intermediate 3-(5-(1,3-dioxolan-2-yl)thiophen-2-yl)benzamide. This was purified by flash column chromatography on silica gel (10% acetone/CH₂Cl₂ as eluant) to give the desired product as an off-white solid, which was deprotected directly according to general procedure B. 3-(5-Formylthiophen-2-yl)benzamide was isolated as a brown solid (73%, 2 steps). ¹H NMR [400 MHz, (CD₃)₂SO] 0.94 (s, 1H), 8.26 (s, 1H), 8.13 (s, 1H), 8.07 (d, J=2.9 Hz, 1H), 7.93 (t, J=8.8 Hz, 2H), 7.81 (d, J=3.1 Hz, 1H), 7.57 (t, J=7.6 Hz, 1H), 7.48 (s, 1H). LRMS (APCI⁺) calcd for C₁₂H₉NO₂S 232 (MH⁺), found 232.

### Example 47

### (E,Z)-3-(5-((5-Oxo-2-thioxoimidazolidin-4-ylidene)methyl)thiophen-2-yl)benzamide (17)

Reaction of 3-(5-formylthiophen-2-yl)benzamide with 2-thiohydantoin according to general procedure D gave the title compound. The resulting solid was isolated by filtration from the reaction mixture as a brown solid (91%), mp (AcOH)>295° C. ¹H NMR [400 MHz, (CD₃)₂SO] δ 12.34 (s, 1H), 11.99 (s, 1H), 8.19 (s, 1H), 8.09 (s, 1H), 7.77-7.86 (m, 3H), 7.71 (d, J=3.8 Hz, 1H), 7.53 (t, J=7.1 Hz, 1H), 7.46 (s, 1H), 6.64 (s, 1H). Anal. (C₁₅H₁₁N₃O₂S₂.0.5H₂O) C, H. N; +0.5.

### Example 48

### (4-(5-(1,3-Dioxolan-2-yl)thiophen-2-yl)phenyl)methanol (114)

5-Bromo-2-thiophenecarboxaldehyde was protected as the cyclic acetal according to a literature procedure¹. This compound and 4-(hydroxymethyl)benzeneboronic acid were reacted according to general procedure A to give the title compound. Purification was carried out by flash column chromatography on silica gel (10% acetone/CH₂Cl₂ as eluant) to give the desired product as a pale yellow solid (66%). ¹H NMR [400 MHz, (CD₃)₂SO] δ 7.60 (d, J=8.3 Hz, 2H), 7.36 (d, J=3.6 Hz, 1H), 7.35 (d, J=8.4 Hz, 2H), 7.20 (d, J=73.8 Hz, 1H), 6.04 (s, 1H), 5.19 (1, J=5.6 Hz, 1H), 4.51 (d, J=5.5 Hz, 2H), 4.10-4.07 (m, 2H), 3.93-4.00 (m, 2H). LRMS (APCI⁺) calcd for C₁₄H₁₅O₃S 263 (MH), found 263. This material contained ca 5% of deprotected aldehyde which was carried into the next step.

### Example 49

### 5-(4-(Hydroxymethyl)phenyl)thiophene-2-carbaldehyde (115)

(4-(5-(1,3-Dioxolan-2-yl)thiophen-2-yl)phenyl)methanol was deprotected according to general procedure B. The title compound was isolated as a yellow solid (100%). ¹H NMR [400 MHz, (CD₃)₂SO] δ 9.90 (s, 1H), 8.03 (d, J=3.9 Hz, 1H), 7.76 (d, J=8.3 Hz, 2H), 7.72 (d, J=4.0 Hz, 1H), 7.42 (d, J=8.4 Hz, 2H), 5.26 (t, J=5.7 Hz, 1H), 4.54 (d, J=5.6 Hz, 2H). LRMS (APCI⁺) calcd for C₁₂H₁₁O₂S 219 (MH⁺), found 219.

### Example 50

### (E,Z)-4-(5-((5-Oxo-2-thioxoimidazolidin-4-ylidene)methyl)thiophen-2-yl)benzyl acetate (18)

Reaction of 5-(4-(hydroxymethyl)phenyl)thiophene-2-carbaldehyde with 2-thiohydantoin according to general procedure D gave the title compound. Purification by flash column chromatography on silica gel (20% THF/hexanes) gave a product which was further purified by recrystallisation from THF/n-pentane. The desired product was obtained as an orange solid (18%), mp (THF/n-pentane) 230-234° C. ¹H NMR [400 MHz, (CD₃)₂SO] δ 12.37 (s, 1H), 11.95 (s, 1H), 7.83 (d, J=4.0 Hz, 1H), 7.72 (d, J=8.3 Hz, 2 H), 7.65 (d, J=4.0 Hz, 1H), 7.44 (d, J=8.4 Hz, 2H), 6.63 (s, 1H), 5.10 (s, 2H), 2.08 (s, 3H). LRMS (APCI⁺) calcd for C₁₇H₁₅N₂O₃S₂ 359 (MH⁺), found 359. Anal. (C₁₇H₁₄N₂O₃S₂) C, H, N.

### Example 51

### Methyl 4-(5-formylthiophen-2-yl)benzoate (116) (Scheme 3)

Methyl 4-bromobenzoate and 5-formyl-2-thiopheneboronic acid were reacted under Suzuki conditions according to general procedure A. Purification of the resulting product by flash column chromatography on silica gel (10% EtOAc/hexanes as eluant) gave the title compound as a white solid (26%). ¹H NMR [400 MHz, (CD₃)₂SO] δ 9.95 (s, 1H), 8.08 (d, J=4.0 Hz, 1H), 8.04 (dt, J=8.6, 1.8 Hz, 2H), 7.95 (dt, J=8.6, 1.8 Hz, 2H), 7.89 (d, J=4.0 Hz, 1H), 3.88 (s, 3 H). LRMS (APCI⁺) calcd for C₁₃H₁₁O₃S 247 (MH⁺), found 247.

### Example 52

### (E,Z)-Methyl 4-(5-((5-oxo-2-thioxoimidazolidin-4-ylidene)methyl)thiophen-2-yl)benzoate (19) (Scheme 3)

Reaction of methyl 4-(5-formylthiophen-2-yl)benzoate with 2-thiohydantoin according to general procedure D gave the title compound. Trituration with dioxane gave the desired product as a bright orange solid (75%), mp (AcOH)>300° C. ¹H NMR [400 MHz, (CD₃)₂SO] δ 12.37 (br s, 1H), 12.00 (br s, 1H), 8.01 (d, J=8.6 Hz, 2H), 7.83-7.88 (m, 3H), 7.79 (d, J=4.0 Hz, 1H), 6.65 (s, 1H), 3.87 (s, 3H). Anal. (C₁₆H₁₂N₂O₃S₂) C, H, N.

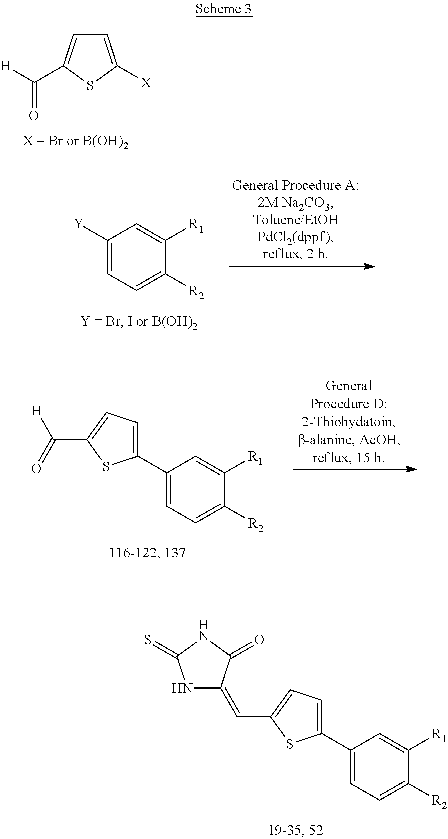

### Example 53

### 5-(4-Acetylphenyl)thiophene-2-carbaldehyde (117)

4-Iodoacetophenone and 5-formyl-2-thiopheneboronic acid were reacted under Suzuki conditions according to general procedure A. Purification of the resulting product by flash column chromatography on silica gel (10% EtOAc/hexanes as eluant) gave the title compound as a pale yellow solid (31%). ¹H NMR [400 MHz, (CD₃)₂SO]δ 9.95 (s, 1H), 8.08 (d, J=4.0 Hz, 1H), 8.04 (d, J=8.6 Hz, 2H), 7.95 (d, J=8.6 Hz, 2H), 7.90 (d, J=4.0 Hz, 1H), 2.16 (s, 3H). LRMS (APCI⁺) calcd for C₁₃H₁₁O₂S 231 (MH⁺), found 231.

### Example 54

### (E,Z)-5-((5-(4-Acetylphenyl)thiophen-2-yl)methylene)-2-thioxoimidazolidin-4-one (20)

Reaction of 5-(4-acetylphenyl)thiophene-2-carbaldehyde with 2-thiohydantoin according to general procedure D gave the title compound. Trituration with dioxane gave the desired product as a pale brown solid (79%), mp (AcOH)>300° C. ¹H NMR [400 MHz, (CD₃)₂SO]δ 12.38 (br s, 1H), 12.00 (br s, 1H), 8.02 (d, J=8.5 Hz, 2H), 7.83-7.89 (m, 3H), 7.81 (d, J=4.0 Hz, 1H), 6.65 (s, 1H), 2.60 (s, 3H). Anal. (C₁₆H₁₂N₂O₂S₂.0.25H₂O) C, H, N.

### Example 55

### Methyl 4-(5-formylthiophen-2-yl)-2-methylbenzoate (118)

Methyl 4-bromo-2-methylbenzoate and 5-formyl-2-thiopheneboronic acid were reacted under Suzuki conditions according to general procedure A. Purification of the resulting product by flash column chromatography on silica gel (10% EtOAc/hexanes as eluant) gave the title compound as a pale yellow solid (19%). ¹H NMR [400 MHz, (CD₃)₂SO] δ 9.94 (s, 1H), 8.07 (d, J=4.0 Hz, 1H), 7.91 (d, J=8.2 Hz, 1H), 7.86 (d, J=4.0 Hz, 1H), 7.78-7.81 (m, 1H), 7.74 (dd, J=8.2, 1.7 Hz, 1H), 3.85 (s, 3H), 2.59 (s, 3H). LAMS (APCI⁺) calcd for C₁₄H₁₃O₃S 261 (MH⁺), found 261.

### Example 56

### (E,Z)-Methyl 2-methyl-4-(5-((5-oxo-2-thioxoimidazolidin-4-ylidene)methyl)thiophen-2-yl)benzoate (21)

Reaction of methyl 4-(5-formylthiophen-2-yl)-2-methylbenzoate with 2-thiohydantoin according to general procedure D gave the title compound. Trituration with dioxane gave the desired product as an orange solid (57%), mp (AcOH) 289° C. (dec.). ¹H NMR [400 MHz, (CD₃)₂SO] δ 12.37 (br s, 1H), 11.99 (br s, 1H), 7.89 (d, J=8.2 Hz, 1H), 7.84 (d, J=3.8 Hz, 1H), 7.76 (d, J=4.0 Hz, 1H), 7.68 (br s, 1H), 7.64 (dd, J=8.2, 1.8 Hz, 1H), 6.65 (s, 1H), 3.84 (s, 3H), 2.58 (s, 3H). Anal. (C₁₇H₁₄N₂O₃S₂) C, H, N.

### Example 57

### (E,Z)-5-((5-(4-Methoxyphenyl)thiophen-2-yl)methylene)-2-thioxoimidazolidin-4-one (22)

Reaction of 5-(4-Methoxyphenyl)thiophene-2-carbaldehyde with 2-thiohydantoin according to general procedure D gave the title compound. Trituration with dioxane gave the desired product as a purple-brown solid (38%), mp (AcOH) 288-293° C. ¹H NMR [400 MHz, (CD₃)₂SO] δ 12.32 (br s, 1H), 11.90 (br s, 1H), 7.79 (d, J=4.0 Hz, 1H), 7.65 J=8.8 Hz, 2H), 7.51 (d, J=4.0 Hz, 1H), 7.02 (d, J=8:9 Hz, 2H), 6.61 (s, 1H), 3.81 (s, 3 H). HRMS (EI⁺) calcd for C₁₅H₁₂N₂O₂S₂ 316.0340 (M⁺), found 316.0338.

### Example 58

### (E,Z)-5-((5-(3-Chlorophenyl)thiophen-2-yl)methylene)-2-thioxoimidazolidin-4-one (23)

Reaction of 5-(3-chlorophenyl)thiophene-2-carbaldehyde with 2-thiohydantoin according to general procedure D gave the title compound as a brown solid (45%), mp (AcOH) 281-283° C. ¹H NMR [400 MHz, (CD₃)₂SO] δ 12.33 (s, 1H), 11.96 (s, 1H), 7.81 (d, J=3.7 Hz, 1H), 7.79 (t, J=1.7 Hz, 1H), 7.73 (d, J=3.9 Hz, 1H), 7.66 (d, J=6.6 Hz, 1H), 7.48 (t, J=7.9 Hz, 1H), 7.40 (d, J=8.0 Hz, 1H), 6.63 (s, 1H). Anal. (C₁₄H₉ClN₂OS₂.0.25H₂O) C, H, N.

### Example 59

### (E,Z)-5-((5-(4-Chlorophenyl)thiophen-2-yl)methylene)-2-thioxoimidazolidin-4-one (24)

Reaction of 5-(4-chlorophenyl)thiophene-2-carbaldehyde with 2-thiohydantoin according to general procedure D gave the title compound as a yellow solid (75%), mp (AcOH)>300° C. ¹H NMR [400 MHz, (CD₃)₂SO] δ 12.36 (s, 1H), 11.96 (s, 1H), 7.81 (d, J=3.9 Hz, 1H), 7.73 (d, J=8.6 Hz, 2H), 7.66 (d, J=3.9 Hz, 1H), 7.49 (d, J=8.6 Hz, 2H), 6.62 (s, 1H). Anal. (C₁₄H₉ClN₂OS₂) C, H, N.

### Example 60

### (E,Z)-5-((5-(Pyridin-4-yl)thiophen-2-yl)methylene)-2-thioxoimidazolidin-4-one (25)

Reaction of 5-(pyridin-4-yl)thiophene-2-carbaldehyde with 2-thiohydantoin according to general procedure D gave the title compound as a brown solid (63%), mp (AcOH)>295° C. ¹H NMR [400 MHz, (CD₃)₂SO] δ 12.45 (s, 1H), 12.05 (s, 1H), 8.64 (d, J=4.9 Hz, 2H), 7.97 (d, J=3.9 Hz, 1H), 7.90 (d, J=3.9 Hz, 1H), 7.77 (d, J=5.9 Hz, 2H), 6.66 (s, 1H). Anal. (C₁₃H₉N₃OS₂.1.5H₂O) C, H, N.

### Example 61

### (E,Z)-5-((5-(Pyridin-3-yl)thiophen-2-yl)methylene)-2-thioxoimidazolidin-4-one (26)

Reaction of 5-(pyridin-3-yl)thiophene-2-carbaldehyde with 2-thiohydantoin according to general procedure D gave the title compound as an orange solid (66%), mp (AcOH)>295° C. ¹H NMR [400 MHz, (CD₃)₂SO] δ 12.34 (s, 1H), 11.98 (s, 1H), 8.95 (d, J=1.8 Hz, 1H), 8.54 (dd, J=1.5, 4.8 Hz, 1H), 8.07-8.10 (m, 1H), 7.86 (d, J=3.9 Hz, 1H), 7.76 (d, J=3.9 Hz, 1H), 7.46-7.49 (m, 1H), 6.64 (s, 1H). Anal. (C₁₃H₉N₃OS₂.0.25H₂O) C, H, N.

### Example 62

### 5-(4-(Methylthio)phenyl)thiophene-2-carbaldehyde (119)

The title compound was prepared by reaction of 5-bromothiophen-2-carboxaldehyde and 4-(methylthio)phenylboronic acid according to general method A, followed by purification by flash column chromatography on silica gel (20% EtOAc/hexanes as eluant). 5-(4-(Methylthio)phenyl)thiophene-2-carbaldehyde was obtained as a yellow solid (94%). ¹H NMR [400 MHz, (CD₃)₂SO] δ 9.88 (s, 1H), 7.72 (d, J=4.0 Hz, 1H), 7.59 (dd, J=2.3, 8.6 Hz, 2H), 7.36 (d, J=4.0 Hz, 1H), 7.28 (dd, J=2.3, 8.5 Hz, 2H), 2.52 (s, 3H). LRMS (APCI⁺) calcd for C₁₂H₁₁OS₂ 235 (MH⁺), found 235.

### Example 63

### ((E,Z)-5-((5-(4-(Methylthio)phenyl)thiophen-2-yl)methylene)-2-thioxoimidazolidin-4-one (27)

Reaction of 5-(4-(methylthio)phenyl)thiophene-2-carbaldehyde with 2-thiohydantoin according to general procedure D gave the title compound as a bright orange solid (80%), mp (AcOH) 289-293° C. ¹H NMR [400 MHz, (CD₃)₂SO] δ 12.34 (s, 1H), 11.96 (s, 1H), 7.79 (d, J=3.9 Hz, 1H), 7.65 (d, J=8.5 Hz, 2H), 7.60 (d, J=4.0 Hz, 1H), 7.32 (td, J=2.3, 8.5 Hz, 2H), 6.61 (s, 1H), 2.53 (s, 3H). Anal. (C₁₅H₁₂N₂OS₃) C, H, N.

### Example 64

### 5-(4-(Methylsulfonyl)phenyl)thiophene-2-carbaldehyde (120)

The title compound was prepared by reaction of 5-bromothiophen-2-carboxaldehyde and 4-(methylsulfonyl)phenylboronic acid according to general method A and was isolated as a brown solid (28%). ¹H NMR [400 MHz, (CD₃)₂SO] δ 9.96 (s, 1H), 8.10 (d, J=4.0 Hz, 1H), 8.07 (d, J=8.6 Hz, 2H), 8.01 (d, J=8.6 Hz, 2H), 7.93 (d, J=4.0 Hz, 1H), 3.26 (s, 3 H). LRMS (APCI⁺) calcd for C₁₂H₁₁O₃S₂ 267 (MH⁺), found 267.

### Example 65

### (E,Z)-5-((5-(4-(Methylsulfonyl)phenyl)thiophen-2-yl)methylene)-2-thioxoimidazolidin-4-one (28)

Reaction of 5-(4-(Methylsulfonyl)phenyl)thiophene-2-carbaldehyde with 2-thiohydantoin according to general procedure D gave the title compound as a brown solid (73%), mp (AcOH) 301-304° C. ¹H NMR [400 MHz, (CD₃)₂SO] δ 12.31 (s, 1H), 11.97 (s, 1H), 7.97 (s, 4H), 7.75-7.85 (m, 2H), 6.64 (s, 1H), 3.24 (s, 3H). HRMS (FAB⁺) calcd for C₁₅H₁₃N₂O₃S₃ 365.0088 (MH⁺), found 365.0089.

### Example 66

### (E,Z)-5-((5-(4-Fluorophenyl)thiophen-2-yl)methylene)-2-thioxoimidazolidin-4-one (29)

Reaction of 5-(4-fluorophenyl)thiophene-2-carbaldehyde with 2-thiohydantoin according to general procedure D gave the title compound as an orange solid (87%), mp (AcOH)>300° C. ¹H NMR [400 MHz, (CD₃)₂SO] δ 12.27 (s, 1H), 11.96 (s, 1H), 7.74-7.81 (m, 3H), 7.60 (d, J=4.0 Hz, 1H), 7.29 (t, J=8.8 Hz, 2H), 6.60 (s, 1H). Anal. (C₁₄H₉FN₂OS₂) C, H, N.

### Example 67

### (E,Z)-5-((5-(3,4-Difluorophenyl)thiophen-2-yl)methylene)-2-thioxoimidazolidin-4-one (30)

Reaction of 5-(3,4-difluorophenyl)thiophene-2-carbaldehyde with 2-thiohydantoin according to general procedure D gave the title compound as an orange solid (75%), mp (AcOH)>300° C. ¹H NMR [400 MHz, (CD₃)₂SO] δ 12.33 (s, 1H), 11.95 (s, 1H), 7.81-7.87 (m, 2H), 7.68 (d, J=4.0 Hz, 1H), 7.48-7.56 (m, 2H), 6.61 (s, 1H). Anal. (C₁₄H₈F₂N₂₀S₂.0.25H₂O) C, H, N.

### Example 68

### (E,Z)-5-((5-(3-Fluorophenyl)thiophen-2-yl)methylene)-2-thioxoimidazolidin-4-one (31)

Reaction of 5-(3-fluorophenyl)thiophene-2-carbaldehyde with 2-thiohydantoin according to general procedure D gave the title compound as an orange solid (75%), mp (AcOH) 285-289° C. ¹H NMR [400 MHz, (CD₃)₂SO] δ 12.35 (s, 1H), 11.96 (s, 1H), 7.82 (d, J=4.0 Hz, 1H), 7.72 (d, J=4.0 Hz, 1H), 7.48-7.60 (m, 3H), 7.19 (t, J=9.0 Hz, 1H), 6.62 (s, 1H). Anal. (C₁₄H₉FN₂OS₂.1.25H₂O) C, N, H; +0.5.

### Example 69

### (E,Z)-5-((5-(4-Bromophenyl)thiophen-2-yl)methylene)-2-thioxoimidazolidin-4-one (32)

Reaction of 5-(4-bromophenyl)thiophene-2-carbaldehyde with 2-thiohydantoin according to general procedure D gave the title compound as a yellow solid (72%), mp (AcOH)>300° C. ¹H NMR [400 MHz, (CD₃)₂SO] δ 12.12 (s, 1H), 11.99 (s, 1H), 7.79 (m, 1H), 7.62-7.68 (m, 5H), 6.60 (s, 1H). HRMS (ESI−TOF) calcd for C₁₄H₈⁷⁹BrN₂OS₂ 362.9267 (M-H), found 362.9256. HRMS (ESI−TOF) calcd for C₁₄H₈⁸¹BrN₂OS₂ 364.9246 (M-H), found 364.9230.

### Example 70

### N-(4-(5-Formylthiophen-2-yl)phenyl)methanesulfonamide (121)

The title compound was prepared by reaction of 5-bromothiophen-2-carboxaldehyde and 4-(methylsulfonamido)phenylboronic acid according to general method A and was isolated as a brown solid (11%). [400 MHz, (CD₃)₂SO] δ 10.07 (s, 1H), 9.89 (s, 1H), 8.01 (d, J=154.0 Hz, 1H), 7.78 (d, J=8.9 Hz, 2H), 7.65 (d, J=4.0 Hz, 1H), 7.27 (d, J=8.7 Hz, 2H), 3.04 (s, 3H). LRMS (APCI⁺) calcd for C₁₂H₁₂NO₃S₂ 282 (MH⁺), found 282.

### Example 71

### (E,Z)-N-(4-(5-((5-Oxo-2-thioxoimidazolidin-4-ylidene)methyl)thiophen-2-yl)phenyl)methanesulfonamide (33)

Reaction of N-(4-(5-formylthiophen-2-yl)phenyl)methanesulfonamide with 2-thiohydantoin according to general procedure D gave the title compound as a brown solid (56%), mp (AcOH)>295° C. ¹H NMR [400 MHz, (CD₃)₂SO] δ 12.31 (s, 1H), 11.94 (s, 1H), 9.93 (s, 1H), 7.80 (d, J=3.8 Hz, 1H), 7.66-7.70 (m, 2H), 7.56 (d, J=4.0 Hz, 1H), 7.26 (d, J=8.7 Hz, 2H), 6.61 (s, 1H), 3.04 (s, 3H). Anal. (C₁₅H₁₃N₃O₃S₃) C, H, N.

### Example 72

### (E,Z)-3-(5-((5-Oxo-2-thioxoimidazolidin-4-ylidene)methyl)thiophen-2-yl)benzonitrile (34)

Reaction of 3-(5-formylthiophen-2-yl)benzonitrile with 2-thiohydantoin according to general procedure D, followed by trituration with Et₂O, gave the title compound as a brown solid (39%), mp (AcOH) 278-281° C. ¹H NMR [400 MHz, (CD₃)₂SO] δ 12.06 (br s, 2H), 8.21 (t, J=1.5 Hz, 1H), 8.00 (ddd, J=9.0, 1.8, 1.0 Hz, 1H), 7.76-7.84 (m, 3H), 7.65 (t, J=7.9 Hz, 1H), 6.60 (s, 1H). Anal. (Cₜ₅H₉N₃OS₂.0.75H₂O) C, H. N; +0.5.

### Example 73

### 5-(4-Hydroxyphenyl)thiophene-2-carbaldehyde (122)

5-Formylthiophen-2-ylboronic acid and 4-bromophenol were reacted according to general procedure A, to give the title compound as a yellow solid (36%). ¹H NMR in agreement with literature values.⁶

### Example 74

### (E,Z)-5-((5-(4-Hydroxyphenyl)thiophen-2-yl)methylene)-2-thioxoimidazolidin-4-one (35)

Reaction of 5-(4-hydroxyphenyl)thiophene-2-carbaldehyde with 2-thiohydantoin according to general procedure D, followed by trituration with Et₂O, gave the title compound as a red-purple solid (88%), mp (AcOH)>300° C. ¹H NMR [400 MHz, (CD₃)₂SO] δ 12.32 (s, 1H), 11.88 (s, 1H), 9.78 (s, 1H), 7.78 (d, J=4.0 Hz, 1H), 7.54 (d, J=8.7 Hz, 2H), 7.44 (d, J=4.0 Hz, 1H), 6.84 (d, J=8.7 Hz, 2H), 6.61 (s, 1H). Anal (C₁₄H₁₀N₂O₄S₂) C, H, N.

### Example 75

### (E,Z)-3-((5-(1-Oxo-1,3-dihydroisobenzofuran-5-yl)thiophen-2-yl)methylene)pyrrolidine-2,5-dione (36) (Scheme 4)

5-(1-Oxo-1,3-dihydroisobenzofuran-5-yl)thiophene-2-carbaldehyde (100 mg, 0.41 mmol) was suspended in MeOH (5 mL), to which was added triphenylphosphoranylidenesuccinimide⁷ (147 mg, 0.41 mmol). This mixture was heated at reflux for 1 h. Upon cooling, the resulting crude product was collected by filtration from the reaction mixture and purified by trituration with acetone. The title compound was isolated as a yellow solid (83 mg, 62%), mp (acetone)>295° C. ¹H NMR [400 MHz, (CD₃)₂SO] δ 11.43 (s, 1H), 8.05 (s, 1H), 7.97 (dd, J=1.5, 8.1 Hz, 1H), 7.90 (d, J=8.1 Hz, 1H), 7.85 (d, J=3.9 Hz, 1H), 7.64 (m, 2H), 5.46 (s, 2H), 3.55 (s, 2H). LRMS (APCI⁺) calcd for C₁₂H₁₂NO₄S 326 (MH⁺), found 326. Anal. (C₁₂H₁₁NO₄S) C, H, N.

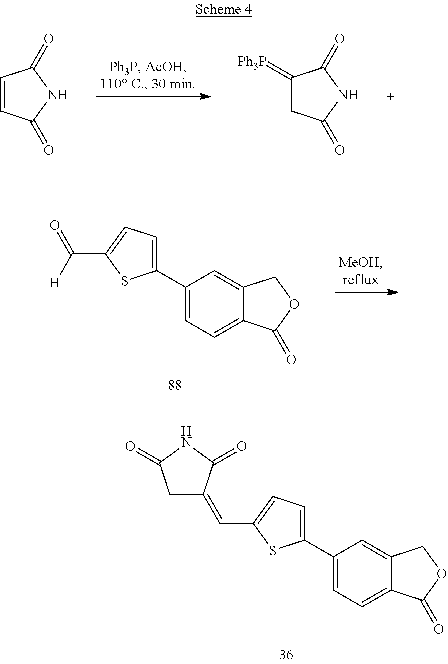

### Example 76

### (E,Z)-1-(4-Methoxybenzyl)-3-((5-(1-oxo-1,3-dihydroisobenzofuran-5-yl)thiophen-2-yl)methylene)pyrrolidin-2-one (123) (Scheme 5)

Diethyl 1-(4-methoxybenzyl)-2-oxopyrrolidin-3-ylphosphonate (prepared by adapting a literature procedure⁸) (233 mg, 0.68 mmol) was dissolved in THF (6 mL), to which was added 18-crown-6 (902 mg, 3.41 mmol). This mixture was cooled to −78° C. under N₂, then a toluene solution of potassium bis(trimethylsilyl)amide (1.50 mL of a 0.5 M solution, 0.75 mmol) added dropwise. After 30 min., a solution of 5-(1-oxo-1,3-dihydroisobenzofuran-5-yl)thiophene-2-carbaldehyde (200 mg, 0.82 mmol) in THF (10 mL) was added and the solution allowed to warns to room temperature and stir for 18 h. NH₄Cl (saturated, 50 mL) was added to the reaction mixture which was extracted with CH₂Cl₂ (3×50 mL). The combined organic fractions were dried (Na₂SO₄) and the solvent removed under reduced pressure to yield a solid which was purified by flash column chromatography on silica gel (5% acetone/CH₂Cl₂ as eluant). The title compound was isolated as a yellow solid (60 mg, 17%). ¹H NMR [400 MHz, (CD₃)₂SO] δ 8.00 (d, J=0.6 Hz, 1H), 7.93 (dd, J=1.5, 8.1 Hz, 1H), 7.87 (d, J=8.1 Hz, 1H), 7.82 (d, J=3.9 Hz, 1H), 7.51 (d, J=4.0 Hz, 1H), 7.44 (t, J=2.7 Hz, 1H), 7.21 (dt, J=2.4, 8.7 Hz, 2H), 6.92 (dt, J=2.5, 8.7 Hz, 2H), 5.44 (s, 2H), 4.48 (s, 2H), 3.74 (s, 3H), 3.41 (t, J=6.2 Hz, 2H), 2.97 (m, 2H). HRMS (FAB⁺) calcd for C₂₅H₂₂NO₄S 432.1270 (MH⁺), found 432.1269.

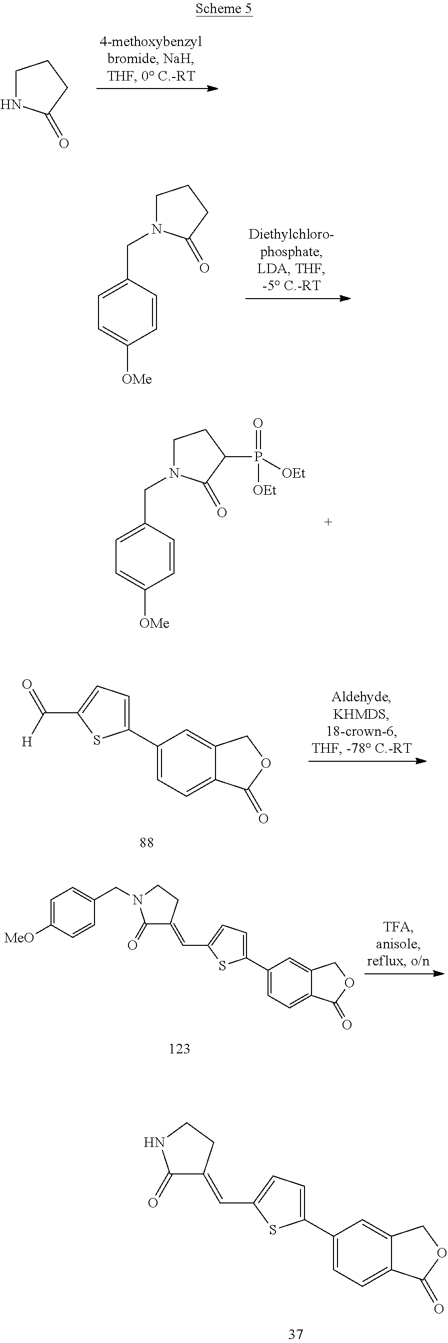 [SMILES: O=C1CCCN1 ; COc1ccc(CN2CCCC2=O)cc1 ; CCOP(=O)(OCC)C1CCN(Cc2ccc(OC)cc2)C1=O ; O=Cc1ccc(-c2ccc3c(c2)COC3=O)s1 ; COc1ccc(CN2CC/C(=C\c3ccc(-c4ccc5c(c4)COC5=O)s3)C2=O)cc1 ; O=C1NCC/C1=C\c1ccc(-c2ccc3c(c2)COC3=O)s1]

### Example 77

### (E,Z)-3-((5-(1-Oxo-1,3-dihydro isobenzofuran-5-yl)thiophen-2-yl)methylene)pyrrolidin-2-one (37) (Scheme 5)

(E,Z)-1-(4-Methoxybenzyl)-3-((5-(1-oxo-1,3-dihydroisobenzofuran-5-yl)thiophen-2-yl)methylene)pyrrolidin-2-one (71 mg, 0.17 mmol) was dissolved in TFA (5 mL), to which was added anisole (5 drops). This mixture was heated at reflux for 15 h. then the solvent removed under reduced pressure. The resulting residue was dissolved in 5% MeOH/CH₂Cl₂ (50 mL) and washed with sat. NaHCO₃ (50 mL). The solution was dried over Na₂SO₄, filtered, and evaporated onto silica prior to purification by flash column chromatography (5% MeOH/CH₂Cl₂ as eluant) to afford the title compound as a yellow solid (39 mg, 71%), m.p. (Et₂O/CH₂Cl₂) 224-227° C. ¹H NMR [400 MHz, (CD₃)₂SO] δ 8.17 (br s, 1H), 3.01 (br s, 1H), 7.93 (dd, J=8.1, 1.5 Hz, 1H), 7.88 (d, J=7.9 Hz, 1 Hz), 7.81 (d, J=3.9 Hz, 1H), 7.48 (d, J=4.0 Hz, 1H), 7.34 (t, J=2.8 Hz, 1H), 5.45 (s, 2H), 3.44 (t, J=6.3 Hz, 2H), 3.01 (td, J=6.4, 2.8 Hz, 2H). LAMS (APCI⁺) calcd for C₁₇H₁₄NO₃S 312 (MH⁺), found 312. Anal. (C₁₂H₁₃NO₃S) C, H, N.

### Example 78

### General Procedure F

4-(5-Formylthiophen-2-yl)-N-methylbenzamide (124) (Scheme 6)

4-(5-Formylthiophen-2-yl)benzoic acid (85 mg, 0.67 mmol) was dissolved in THF (5 mL), to which was added pyridine (289 mg, 3.66 mmol) and pentafluorophenyl trifluoroacetate (512 mg, 1.83 mmol). This mixture was stirred for 2 h. at R.T. The solvent was removed under reduced pressure to give a crude solid which was dissolved in EtOAc (50 mL) and washed with 1 M HCl (2×50 mL), water (50 mL), sat. NaHCO₃ (2×50 mL), brine (50 mL) and dried (Na₂SO₄). The solvent was removed under reduced pressure and the resulting solid purified by filtration through a plug of silica gel (20% EtOAc/hexanes as eluant). The resulting yellow solid was dissolved in THF (5 mL), to which was added methylamine solution (0.8 mL of a 2 M solution in methanol). Complete reaction of the ester was observed by TLC after 1 h. at R.T., then 1 M HCl (5 mL) was added to hydrolyze any imine. After stirring at R.T. for an additional hour, the reaction mixture was diluted with water (20 mL) and extracted with EtOAc (2×20 mL). The combined organic extracts were dried (Na₂SO₄) and the solvent removed under reduced pressure to afford a crude product which was purified by trituration with Et₂O, giving the title compound as a pale yellow solid (72 mg, 80%). ¹H NMR [400 MHz, (CD₃)₂SO] δ 9.93 (s, 1H), 8.47-8.53 (br m, 1H), 8.07 (d, J=4.0 Hz, 1H), 7.93 (d, J=8.7 Hz, 2H), 7.89 (d, J=8.7 Hz, 2H), 7.84 (d, J=4.0 Hz, 1H), 2.80 (d, J=4.5 Hz, 3H). LRMS (AFCI⁺) calcd for C₁₃H₁₂NO₂S 246 (MH⁺), found 246.

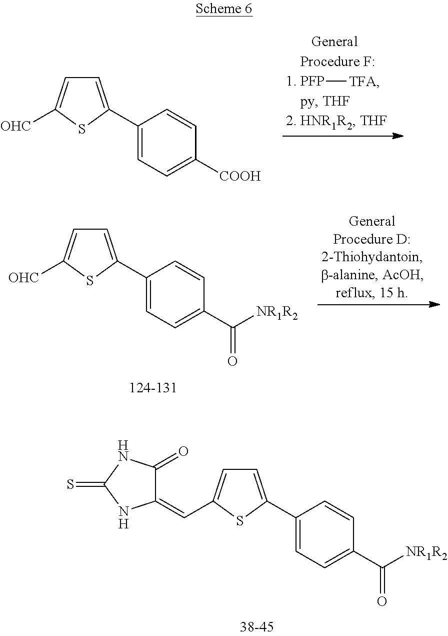

### Example 79

### (E,Z)-N-Methyl-4-(5-((5-oxo-2-thioxoimidazolidin-4-ylidene)methyl)thiophen-2-yl)benzamide (38) (Scheme 6)

Reaction of 4-(5-formylthiophen-2-yl)-N-methylbenzamide with 2-thiohydantoin according to general procedure D gave the title compound which was obtained by filtration from the reaction mixture as a dark orange solid (76%), mp (AcOH)>300° C. ¹H NMR [400 MHz, (CD₃)₂SO] δ 12.37 (br s, 1H), 11.97 (br s, 1H), 8.47 (br q, J=4.5 Hz, 1H), 7.90 (d, J=8.5 Hz, 2H), 7.84 (d, J=4.0 Hz, 1H), 7.80 (d, J=8.5 Hz, 2H), 7.74 (d, J=4.0 Hz, 1H), 2.79 (d, J=4.5 Hz, 3H). LRMS (APCI⁻) calcd for C₁₆H₁₂N₃O₂S₂ 342 (M-H), found 342. Anal. (C₁₆H₁₃N₃O₂S₂) C, H, N.

### Example 80

### 4-(5-Formylthiophen-2-yl)-N,N-dimethylbenzamide (125)

4-(5-Formylthiophen-2-yl)benzoic acid was reacted with pentafluorophenyl trifluoroacetate, followed by 40% dimethylamine in water according to general procedure F. The crude product was purified by trituration with Et₂O to give the title compound as a pale yellow solid (100%). ¹H NMR [400 MHz, (CD₃)₂SO] δ 9.93 (s, 1H), 8.06 (d, J=4.0 Hz, 1H), 7.86 (d, J=8.5 Hz, 2H), 7.81 (d, J=4.0 Hz, 1H), 7.51 (d, J=8.5 Hz, 2H), 2.51 (s, 6H). LRMS (APCI⁺) calcd for C₁₄H₁₄NO₂S 260 (MH⁺), found 260.

### Example 81

### (E,Z)-N,N-Dimethyl-4-(5-((5-oxo-2-thioxoimidazolidin-4-ylidene)methyl)thiophen-2-yl)benzamide (39)

Reaction of 4-(5-formylthiophen-2-yl)-N,N-dimethylbenzamide with 2-thiohydantoin according to general procedure D gave the title compound which was obtained by filtration from the reaction mixture as a brown-orange solid (62%), mp (AcOH)>295° C. ¹H NMR [400 MHz, (CD₃)₂SO] δ 12.38 (br s, 1H), 11.97 (br s, 1H), 7.85 (d, J=3.8 Hz, 1H), 7.77 (d, J=8.4 Hz, 2H), 7.71 (d, J=4.0 Hz, 1H), 7.48 (d, J=2 H), 6.64 (s, 1H), 2.97 (s, 6H). LRMS (APCI⁻) calcd for C₁₇H₁₄N₃O₂S₂ 356 (M-H), found 356. Anal. (C₁₇H₁₅N₃O₂S₂) C, H, N.

### Example 82

### N-(2,3-Dihydroxypropyl)-4-(5-formylthiophen-2-yl)benzamide (126)

4-(5-Formylthiophen-2-yl)benzoic acid was reacted with pentafluorophenyl trifluoroacetate, followed by 3-aminopropane-1,2-diol according to general procedure F. In this case the reaction mixture was treated with 1 M HCl to hydrolyze any imine prior to isolation. The title compound was isolated as a pale yellow solid (45%). ¹H NMR [400 MHz, (CD₃)₂SO] δ 9.94 (s, 1H), 8.47 (t, J=5.5 Hz, 1H), 8.07 (d, J=4.0 Hz, 1H), 7.96 (d, J=8.5 Hz, 2 H), 7.90 (d, J=8.5 Hz, 2H), 7.85 (d, J=4.0 Hz, 1H), 4.78 (d, J=5.0 Hz, 1H), 4.54 (t, J=5.8 Hz, 1H), 3.71-3.60 (m, 1H), 3.48-3.32 (m, 3H), 3.23 (m, 1H). LRMS (APCI⁺) calcd for C₁₅H₁₆NO₄S 306 (MH⁺), found 306.

### Example 83

### (E,Z)-N-(2,3-Dihydroxypropyl)-4-(5-((5-oxo-2-thioxoimidazolidin-4-ylidene)methyl)thiophen-2-yl)benzamide (40)

Reaction of N-(2,3-dihydroxypropyl)-4-(5-formylthiophen-2-yl)benzamide with 2-thiohydantoin according to general procedure D gave the title compound which was obtained by filtration from the reaction mixture as an orange powder (32%), mp (AcOH) 287° C. dec. ¹H NMR [400 MHz, (CD₃)₂SO] δ 12.38 (s, 1H), 11.98 (s, 1H), 8.43 (t, J=5.7 Hz, 1H), 7.93 (d, J=8.6 Hz, 2H), 7.85 (d, J=4.0 Hz, 2H), 7.80 (d, J=8.6 Hz, 2H), 7.76 (d, J=4.0 Hz, 2H), 6.65 (s, 1H), 4.78 (d, J=5.0 Hz, 1H), 4.54 (t, J=5.8 Hz, 1H), 3.66 (tt, J=10.5, 5.3 Hz, 1H), 3.36 (t, J=5.6 Hz, 1H), 3.21 (m, 1H). HRMS (ESI−TOF) calcd for C₁₈H₁₈N₃O₄S₂ 404.0733 (MH⁺), found 404.0713. Anal. (C₁₈H₁₇N₃O₄S₂) C, H, N.

### Example 84

### 4-(5-Formylthiophen-2-yl)-N-(2-hydroxypropyl)benzamide (127)

4-(5-Formylthiophen-2-yl)benzoic acid was reacted with pentafluorophenyl trifluoroacetate, followed by 1-aminopropan-2-ol according to general procedure F. In this case the reaction mixture was treated with 1 M HCl to hydrolyze any imine prior to isolation. The title compound was isolated as a pale yellow solid (58%). ¹H NMR [400 MHz, (CD₃)₂SO] δ 9.94 (s, 1H), 8.54 (t, J=5.7 Hz, 1H), 8.08 (d, J=4.0 Hz, 1H), 7.96 (d, J=8.6 Hz, 2H), 7.91 (d, J=8.6 Hz, 2H), 7.86 (d, J=4.0 Hz, 1H), 3.84-3.75 (m, 1H), 3.22 (ddd, J=9.9, 6.8, 4.0 Hz, 2H), 1.07 (d, J=8.0 Hz, 3H); OH too broad to integrate. LRMS (APCI⁺) calcd for C₁₅H₁₆NO₃S 290 (MH), found 290.

### Example 85

### (E,Z)-N-(2-Hydroxypropyl)-4-(5-((5-oxo-2-thioxoimidazolidin-4-ylidene)methyl)thiophen-2-yl)benzamide (41)

Reaction of 4-(5-formylthiophen-2-yl)-N-(2-hydroxypropyl)benzamide with 2-thiohydantoin according to general procedure D gave the title compound which was obtained by filtration from the reaction mixture as an orange powder (26%), mp (AcOH) 277° C. dec. ¹H NMR [400 MHz, (CD₃)₂SO] Observe E- and Z-isomers separately. δ 12.44 (s, 0.5H), 12.33 (s, 0.5H), 12.16 (s, 0.5H), 12.03 (s, 0.5H), 8.49 (dt, J=5.9, 5.7, 3.4 Hz, 1H), 7.95-7.90 (m, 2H), 7.87 (d, J=4.0 Hz, 0.5H), 7.83-7.75 (m, 3H), 7.68 (d, J=4.0 Hz, 0.5H), 6.84 (s, 0.5H), 6.66 (s, 0.5H), 4.88-4.65 (m, 1H), 3.97-3.61 (m, 1H), 3.21 (dd, J=9.8, 6.0 Hz, 2H), 1.07 (d, J=8.0 Hz, 3H). Anal. (C₁₈H₁₇N₃O₃S₂) C, H, N.

### Example 86

### 4-(5-Formylthiophen-2-yl)-N-(2-hydroxyethyl)benzamide (128)

4-(5-Formylthiophen-2-yl)benzoic acid was reacted with pentafluorophenyl trifluoroacetate, followed by ethanolamine according to general procedure F. In this case the reaction mixture was treated with 1 M HCl to hydrolyze any imine prior to isolation. The title compound was isolated as a pale yellow solid (40%). ¹H NMR [400 MHz, (CD₃)₂SO] δ 9.94 (s, 1H), 8.52 (t, J=5.50 Hz, 1H), 8.07 (d, J=4.0 Hz, 1H), 7.96 (d, J=8.7 Hz, 2H), 7.90 (d, J=8.7 Hz, 2H), 7.85 (d, J=4.0 Hz, 1H), 4.71 (s, 1H), 3.53 (t, J=6.2 Hz, 2H), 3.35 (dd, J=12.0, 6.1 Hz, 2H). LRMS (APCI⁺) calcd for C₁₄H₁₄NO₃S 276 (MH⁺), found 276.

### Example 87

### (E,Z)-N-(2-Hydroxyethyl)-4-(5-((5-oxo-2-thioxoimidazolidin-4-ylidene)methyl)thiophen-2-yl)benzamide (42)

Reaction of 4-(5-formylthiophen-2-yl)-N-(2-hydroxyethyl)benzamide with 2-thiohydantoin according to general procedure D gave the title compound which was obtained by filtration from the reaction mixture as an orange powder (61%), mp (AcOH) 281-284° C. ¹H NMR [400 MHz, (CD₃)₂SO] δ 12.38 (s, 1H), 11.97 (s, 1H), 8.47 (t, J=5.6 Hz, 1H), 7.93 (d, J=8.5 Hz, 2H), 7.85 (d, J=4.0 Hz, 1H), 7.80 (d, J=8.5 Hz, 2H), 7.75 (d, J=4.0 Hz, 1H), 6.65 (s, 1H), 4.70 (t, J=5.6 Hz, 1H), 3.53 (q, J=6.0 Hz, 2H), 3.35 (q, J=6.0 Hz, 2H). FIRMS (ESI−TOF) calcd for C₁₂H₁₄N₃O₃S₂ 372.0477 (M-H), found 372.0465. Anal. (C₁₇H₁₅N₃O₃S₂) C, H, N.

### Example 88

### 4-(5-Formylthiophen-2-yl)-N-(3-morpholinopropyl)benzamide (129)

4-(5-Formylthiophen-2-yl)benzoic acid was reacted with pentafluorophenyl trifluoroacetate, followed by 4-(3-aminopropyl)morpholine according to general procedure F. In this case the reaction mixture was treated with 1 M HCl to hydrolyze any imine prior to isolation.

The title compound was isolated as a pale yellow solid (100%). ¹H NMR [400 MHz, (CD₃)₂SO] δ 9.94 (s, 1H), 8.56 (t, J=5.6 Hz, 1H), 8.07 (d, J=4.0 Hz, 1H), 7.93 (d J=8.8 Hz, 2H), 7.90 (d, J=8.8 Hz, 2H), 7.85 (d, J=4.0 Hz, 1H), 3.62-3.54 (m, 4H), 3.31 (dd, J=12.7, 6.9 Hz, 4H), 2.44-2.30 (m, 4H), 1.71 (m, 2H). LRMS (APCI⁺) calcd for C₁₉H₂₃N₂O₃S 359 (MH⁺), found 359.

### Example 89

### (E,Z)-N-(3-Morpholinopropyl)-4-(5-((5-oxo-2-thioxoimidazolidin-4-ylidene)methyl)thiophen-2-yl)benzamide (43)

Reaction of 4-(5-formylthiophen-2-yl)-N-(3-morpholinopropyl)benzamide with 2-thiohydantoin according to general procedure D gave the title compound which was obtained by filtration from the reaction mixture as an orange powder (9%), mp (AcOH) 287° C. dec. ¹H NMR [400 MHz, (CD₃)₂SO] Observe E- and Z- isomers separately. δ 12.18 (m, 2H), 8.52 (t, J=5.5 Hz, 1H), 7.90 (d, J=8.5 Hz, 2 H), 7.80 (m, 3H), 7.74 (d, J=4.0 Hz, 1H), 6.83 (s, 0.2 Hz), 6.62 (s, 0.8 Hz), 3.54-3.61 (m, 4H), 3.32 (m, 2H), 2.35 (m, 6H), 1.70 (m, 2H). HRMS (ESI−TOF) calcd for C₂₂H₂₅N₄O₃S₂ 457.1363 (MH⁺), found 457.1369.

### Example 90

### 4-(5-Formylthiophen-2-yl)-N-(3-hydroxypropyl)benzamide (130)

4-(5-Formylthiophen-2-yl)benzoic acid was reacted with pentafluorophenyl trifluoroacetate, followed by 3-aminopropanol according to general procedure F. In this case the reaction mixture was treated with 1 M HCl to hydrolyze any imine prior to isolation. The title compound was isolated as a pale yellow solid (72%). ¹H NMR [400 MHz, (CD₃)₂SO] δ 9.94 (s, 1H), 8.52 (t, J=5.5 Hz, 1H), 8.07 (d, J=4.0 Hz, 1H), 7.93 (d, J=8.7 Hz, 2 H), 7.89 (d, J=8.7 Hz, 2H), 7.85 (d, J=4.0 Hz, 1H), 4.45 (t, J=5.2 Hz, 1H), 3.48 (dd, J=11.4, 6.2 Hz, 2H), 3.33 (dd, J=12.7, 7.1 Hz, 2H), 1.69 (m, 2H). LRMS (APCI⁺) calcd for C₁₅H₁₆NO₃S⁺ 290 (MH⁺), found 359.

### Example 91

### (E,Z)-N-(3-Hydroxypropyl)-4-(5-((5-oxo-2-thioxoimidazolidin-4-ylidene)methyl)thiophen-2-yl)benzamide (44)

Reaction of 4-(5-formylthiophen-2-yl)-N-(3-hydroxypropyl)benzamide with 2-thiohydantoin according to general procedure D gave the title compound which was obtained by filtration from the reaction mixture as an orange powder (55%), mp (AcOH) 282-285° C. ¹H NMR [400 MHz, (CD₃)₂SO] Observe E- and Z-isomers separately. δ 12.39 (s, 0.5H), 12.28 (s, 0.5H), 12.10 (s, 0.5H), 11.97 (s, 0.5H), 8.51-8.43 (m, 1H), 7.95-7.88 (m, 2H), 7.86 (d, J=4.1 Hz, 1H), 7.83-7.73 (m, 2H), 7.67 (d, J=4.0 Hz, 1H), 6.83 (s, 0.5H), 6.65 (s, 0.5H), 4.45 (t, J=4.2, 4.2 Hz, 1H), 3.48 (dd, J=9.9, 5.8 Hz, 2H), 3.33 (dd, J=13.1, 6.8 Hz, 2H), 1.69 (m, 2H). Anal. (C₁₉H₁₇N₃O₃S₂) C, H, N.

### Example 92

### 4-(5-Formylthiophen-2-yl)-N-(2-morpholinoethyl)benzamide (131)

4-(5-Formylthiophen-2-yl)benzoic acid was reacted with pentafluorophenyl trifluoroacetate, followed by 4-(2-aminoethyl)morpholine according to general procedure F. In this case the reaction mixture was treated with 1 M HCl to hydrolyze any imine prior to isolation. The title compound was isolated as a pale yellow solid (100%). ¹H NMR [400 MHz, (CD₃)₂SO] δ 9.94 (s, 1H), 8.49 (t, J=5.6 Hz, 1H), 8.07 (d, J=4.0 Hz, 1H), 7.93 (d, J=8.8 Hz, 2H). 7.90 (d, J=8.8 Hz, 2H), 7.84 (d, J=4.0 Hz, 1H), 3.51-3.66 (m, 4H), 3.41 (dd, J=12.9, 6.7 Hz, 4H), 2.45 (m, 4H). LRMS (APCI⁺) calcd for C₁₈H₂₁N₂O₃S (MH⁺) 345, found 345.

### Example 93

### (E,Z)-N-(2-Morpholinoethyl)-4-(5-((5-oxo-2-thioxoimidazolidin-4-ylidene)methyl)thiophen-2-yl)benzamide (45)

Reaction of 4-(5-formylthiophen-2-yl)-N-(2-morpholinoethyl)benzamide with 2-thiohydantoin according to general procedure D gave the title compound which was obtained by filtration from the reaction mixture as an orange powder (38%), mp (AcOH) 248-252° C. ¹H NMR [400 MHz, (CD₃)₂SO] δ 12.32 (s, 1H), 11.94 (s, 1H), 8.45 (t, J=5.5 Hz, 1H), 7.90 (d, J=8.5 Hz, 2H), 7.84 (d, J=3.9 Hz, 1H), 7.80 (d, J=8.5 Hz, 2H), 7.75 (d, J=4.0 Hz, 1H), 6.63 (s, 1H), 3.51-3.67 (m, 4H), 3.41 (dd, J=12.8, 6.6 Hz, 4H), 2.45 (m 4H). Anal. (C₂₁H₂₂N₄O₃S₂1.5 H₂O) C, H, N.

### Example 94

### 5-(1-Oxoisoindolin-5-yl)thiophene-2-carbaldehyde (132) (Scheme 1)

5-Bromo-2-thiophenecarboxaldehyde was protected as the cyclic acetal according to a literature procedure¹. This compound was then reacted with 5-(4,4,5,5-tetramethyl-1,3,2-dioxaborolan-2-yl)isoindolin-1-one³ according to general procedure A. Purification of the resulting product by flash column chromatography on silica gel (2% MeOH/CH₂Cl₂ as eluant), followed by trituration with Et₂O, gave 5-(5-(1,3-dioxolan-2-yl)thiophen-2-yl)isoindolin-1-one as a pale yellow solid, containing ˜10% of the deprotected aldehyde. This solid was therefore completely deprotected according to general procedure B. Purification was carried out by trituration with CH₂Cl₂, giving the title compound as a pale yellow solid (52% over both steps). ¹H NMR [400 MHz, (CD₃)₂SO] δ 9.94 (s, 1H), 8.64 (br s, 1H), 8.08 (d, J=4.0 Hz, 1H), 8.02 (br s, 1H), 7.92 (dd, J=7.9, 1.5 Hz, 1H), 7.85 (d, J=4.0 Hz, 1H), 7.75 (d, J=7.9 Hz, 1H), 3.54 (s, 2H). LRMS (APCI⁺) calcd for C₁₃H₁₀NO₂S 244 (MH⁺), found 244.

### Example 95

### (E,Z)-5-(5-((5-Oxo-2-thioxoimidazolidin-4-ylidene)methyl)thiophen-2-yl)isoindolin-1-one (46)

Reaction of 5-(1-oxoisoindolin-5-yl)thiophene-2-carbaldehyde with 2-thiohydantoin according to general procedure D gave the title compound. Trituration with dioxane gave the desired product as an orange solid (57%), mp (DMSO/H₂O)>300° C. ¹H NMR [400 MHz, (CD₃)₂SO] δ 12.38 (s, 1H), 11.98 (s, 1H), 8.57 (s, 1H), 7.91 (s, 1H), 7.86 (d, J=3.9 Hz, 1H), 7.83 (dd, J=1.4, 7.9 Hz, 1H), 7.76 (d, J=3.9 Hz, 1H), 7.71 (d, J=7.9 Hz, 1H), 6.65 (s, 1H), 4.43 (s, 2H). Anal. (C₁₆H₁₁N₃O₂S₂.1.5H₂O) C, H, N; +0.5.

### Example 96

### (E,Z)-5-((5-(1-Oxoisoindolin-5-yl)thiophen-2-yl)methylene)imidazolidine-2,4-dione (47)

Reaction of 5-(1-oxoisoindolin-5-yl)thiophene-2-carbaldehyde with hydantoin according to general procedure D gave the title compound as a yellow-brown solid (41%), mp (DMSO/H₂O)>295° C. ¹H NMR [400 MHz, (CD₃)₂SO] δ 11.27 (s, 1H), 10.39 (s, 1H), 8.55 (s, 1H), 7.87 (s, 1H), 7.80 (dd, J=1.4, 7.9 Hz, 1H), 7.71 (m, 2H), 7.62 (d, J=4.2 Hz, 1H), 6.59 (s, 1H), 4.42 (s, 2H). Anal. (C₁₆H₁₁N₃O₃S.1.25H₂O) C, H, N.

### Example 97

### 4-(1-Oxoisoindolin-5-yl)benzaldehyde (133) (Scheme 2)

4-Bromobenzaldehyde was protected as the dimethyl acetal according to a literature procedure⁵, then reacted with 5-(4,4,5,5-tetramethyl-1,3,2-dioxaborolan-2-yl)isoindolin-1-one according to general procedure A. Purification by flash column chromatography on silica gel (EtOAc as eluant) gave the desired product as a cream solid (84%), which was deprotected directly to the corresponding aldehyde according to general procedure E. The title compound was isolated as a cream solid (78%). ¹H NMR [400 MHz, (CD₃)₂SO] δ 10.08 (s, 1H), 8.60 (br s, 1H), 8.03 (d, J=8.5 Hz, 2H), 7.95-8.00 (m, 3H), 7.84-7.89 (m, 1H), 7.78 (d, J=7.9 Hz, 1H), 4.46 (s, 2H). LRMS (APCI⁺) calcd for C₁₅H₁₂NO₂ 238 (MH⁺), found 238.

### Example 98

### (E,Z)-5-(4-((5-Oxo-2-thioxoimidazolidin-4-ylidene)methyl)phenyl)isoindolin-1-one (48)

Reaction of 4-(1-oxoisoindolin-5-yl)benzaldehyde with 2-thiohydantoin according to general procedure D gave the title compound which was obtained by filtration from the reaction mixture as a yellow solid (66%), mp (AcOH)>300° C. ¹H NMR [400 MHz, (CD₃)₂SO] δ 12.38 (br s, 1H), 12.22 (br s, 1H), 8.56 (br s, 1H), 7.93 (s, 1H), 7.89 (d, J=8.5 Hz, 2H), 7.84 (dd, J=8.0, 1.3 Hz, 1H), 7.80 (d, J=8.5 Hz, 2H), 7.76 (d, J=7.9 Hz, 1H), 6.54 (s, 1H), 4.45 (s, 2H). LRMS (APCI⁺) calcd for C₁₈H₁₂N₃O₂S 334 (M-H), found 334. Anal. (C₁₈H₁₃N₃O₂S.0.5AcOH) C, H, N.

### Example 99

### 3-(1-Oxoisoindolin-5-yl)benzaldehyde (134)

3-Bromobenzaldehyde was protected as the dimethyl acetal according to a literature procedure⁵, then reacted with 5-(4,4,5,5-tetramethyl-1,3,2-dioxaborolan-2-yl)isoindolin-1-one according to general procedure A. Purification by flash column chromatography on silica gel (EtOAc as eluant) gave the desired product as a cream solid (64%), which was deprotected directly to the corresponding aldehyde according to general procedure E. The title compound was isolated as a cream solid (87%). ¹H NMR [400 MHz, (CD₃)₂SO] δ 10.12 (s, 1H), 8.58 (br s, 1H), 8.28 (t, J=1.6 Hz, 1H), 8.08 (ddd, J=7.8, 1.9, 1.2 Hz, 1H), 7.93-7.98 (m, 2H), 7.85 (dd, J=7.9, 1.5 Hz, 1H), 7.78 (d, J=7.9 Hz, 1H), 7.74 (t, J=7.7 Hz, 1H), 4.46 (s, 2H). LRMS (APCI⁺) calcd for C₁₅H₁₂NO₂ 238 (MH⁺), found 238.

### Example 100

### (E,Z)-5-(3-((5-Oxo-2-thioxoimidazolidin-4-ylidene)methyl)phenyl)isoindolin-1-one (49)

Reaction of 3-(1-oxoisoindolin-5-yl)benzaldehyde with 2-thiohydantoin according to general procedure D gave the title compound which was obtained by filtration from the reaction mixture as a yellow solid (81%), mp (AcOH)>300° C. ¹H NMR [400 MHz, (CD₃)₂SO] δ 12.38 (br s, 1H), 12.30 (br s, 1H), 8.56 (br s, 1H), 8.01 (s, 1H), 7.93 (s, 1H), 7.85 (dd, J=7.9, 1.3 Hz, 1H), 7.74-7.79 (m, 2H), 7.72 (d, J=8.1 Hz, 1H), 7.55 (t, J=7.8 Hz, 1H), 6.59 (s, 1H), 4.45 (s, 2H). LRMS (APCI⁻) calcd for C₁₈H₁₂N₃O₂S 334 (M-H), found 334. Anal. (C₁₈H₁₃N₃O₂S.0.5AcOH) C, H, N.

### Example 101

### 4-(1-Oxoisoindolin-5-yl)thiophene-2-carbaldehyde (135)

4-Bromo-2-thiophenecarboxaldehyde was protected as the dimethyl acetal according to a literature procedure⁵, then reacted with 5-(4,4,5,5-tetramethyl-1,3,2-dioxaborolan-2-yl)isoindolin-1-one according to general procedure A. In this case deprotection of the dimethyl acetal occurred in situ, affording the aldehyde directly. Purification by flash column chromatography on silica gel (EtOAc as eluant) gave the desired product as a cream solid (74%). ¹H NMR [400 MHz, (CD₃)₂SO] δ 10.00 (d, J=1.2 Hz, 1H), 8.53-8.59 (m, 3H), 7.98 (s, 1H), 7.89 (dd, J=7.9, 1.4 Hz, 1H), 7.74 (d, J=7.9 Hz, 1H), 4.43 (s, 2H). LRMS (APCI⁺) calcd for C₁₃H₁₀NO₂S 244 (MH⁺), found 244.

### Example 102

### (E,Z)-5-(5-((5-Oxo-2-thioxoimidazolidin-4-ylidene)methyl)thiophen-3-yl)isoindolin-1-one (50)

Reaction of 4-(1-oxoisoindolin-5-yl)thiophene-2-carbaldehyde with 2-thiohydantoin according to general procedure D gave the title compound which was obtained by filtration from the reaction mixture as a yellow solid (58%), mp (AcOH)>300° C. ¹H NMR [400 MHz, (CD₃)₂SO] δ 12.41 (br s, 1H), 12.27 (br s, 1H), 8.55 (br s, 1H), 8.32 (s, 1H), 8.24 (d, J=1.0 Hz, 1H), 8.01 (s, 1H), 7.97 (dd, J=7.9, 1.3 Hz, 1H), 7.75 (d, J=7.9 Hz, 1H), 6.55 (s, 1H), 4.45 (s, 2H). HRMS (FAB⁺) calcd for C₁₆H₁₂N₃O₂S₂ 342.0371 (MH⁺), found 342.0372.

### Example 103

### 5-(6-(Dimethoxymethyl)pyridin-3-yl)isoindolin-1-one (136)

5-Bromopyridinecarboxaldehyde was protected as the dimethyl acetal according to a literature procedure⁵, then reacted with 5-(4,4,5,5-tetramethyl-1,3,2-dioxaborolan-2-yl)isoindolin-1-one according to general procedure A, to give the title compound. Purification by flash column chromatography on silica gel (5% MeOH/CH₂Cl₂ as eluant) gave the desired product as a cream solid (63%). ¹H NMR [400 MHz, (CD₃)₂SO] δ 8.92 (dd, J=2.4, 0.7 Hz, 1H), 8.59 (br s, 1H), 8.19 (dd, J=8.2, 2.4 Hz, 1H), 7.94 (d, J=0.7 Hz, 1H), 7.84 (dd, J=7.9, 1.5 Hz, 1H), 7.78 (d, J=7.7 Hz, 1H), 7.61 (d, J=8.2 Hz, 1H), 5.36 (s, 1H), 4.48 (s, 2H), 3.35 (s, 6H). LRMS (APCI⁺) calcd for C₁₆H₁₇N₂O₃ 285 (MH⁺), found 285.

### Example 104

### (E,Z)-5-(6((5-Oxo-2-thioxoimidazolidin-4-ylidene)methyl)pyridin-3-yl)isoindolin-1-one (51)

5-(6-(Dimethoxymethyl)pyridin-3-yl)isoindolin-1-one was deprotected according to general method E to give the corresponding aldehyde as an off-white solid. This material was then used directly in the next step to react with 2-thiohydantoin according to general procedure D. The title compound was obtained by filtration from the reaction mixture as a dark green solid (50% over 2 steps), mp (AcOH)>300° C. ¹H NMR. [400 MHz, (CD₃)₂SO] δ 12.53 (s, 1H), 11.50 (s, 1H), 9.13 (d, J=2.4 Hz, 1H), 8.62 (s, 1H), 8.25 (dd, J=8.2, 2.4 Hz, 1H), 7.99 (s, 1H), 7.89 (dd, J=8.0, 1.3 Hz, 1H), 7.85 (d, J=8.1 Hz, 1H), 7.80 (d, J=7.9 Hz, 1H), 6.68 (s, 1H), 4.46 (s, 2H). LRMS (APCI⁻) calcd for C₁₇H₁₁N₄O₂S 335 (M-H), found 335. Anal. (C₁₇H₁₂N₄O₂S.0.5H₂O) C, H, N.

### Example 105

### 5-(2-Isopropyl-1-oxoisoindolin-5-yl)thiophene-2-carbaldehyde (137) (Scheme 3)

5-Bromo-2-isopropylisoindolin-1-one (prepared according to a literature procedure⁹) was reacted with 5-formylthiophen-2-ylboronic acid according to general procedure A. The crude material was purified by flash column chromatography on silica gel (50% EtOAc/hexanes) to give the title compound as a pale yellow solid (44%). ¹H NMR [400 MHz, (CD₃)₂SO] δ 9.94 (s, 1H), 8.08 (d, J=4.0 Hz, 1H), 8.03 (d, J=0.8 Hz, 1H), 7.92 (dd, J=7.9, 1.6 Hz, 1H), 7.85 (d, J=4.0 Hz, 1H), 7.74 (d, J=7.6 Hz, 1H), 4.50 (s, 2H), 4.48-4.39 (m, 1H), 1.26 (s, 3H), 1.24 (s, 3H). LRMS (APCI⁺) calcd for C₁₆H₁₆NO₂S 286 (MH⁺), found 286.

### Example 106

### (E,Z)-2-Isopropyl-5-(5-((5-oxo-2-thioxoimidazolidin-4-ylidene)methyl)thiophen-2-yl)isoindolin-1-one (52)

Reaction of 5-(2-isopropyl-1-oxoisoindolin-5-yl)thiophene-2-carbaldehyde with 2-thiohydantoin according to general procedure D gave the title compound which was obtained by filtration from the reaction mixture as a red powder (23%), mp (AcOH) 285-290° C. ¹H NMR [400 MHz, (CD₃)₂SO] Observe E- and Z-isomers separately. δ 12.39 (s, 1H), 11.99 (s, 1H), 7.92 (d, J=0.70 Hz, 1H), 7.87 (d, J=4.0 Hz, 1H), 7.83 (dd, J=8.0, 1.5 Hz, 1H), 7.76 (d, J=4.0 Hz, 1H), 7.71 (d, J=7.9 Hz, 1H), 6.65 (s, 1H), 4.48 (s, 2H), 4.43 (td, J=13.5, 6.8 Hz, 1H), 1.26 (s, 3H), 1.24 (s, 3 H). HRMS (ESI−TOF) calcd for C₁₉H₁₈N₃O₂S₂ 384.0835 (MH⁺), found 384.0821.

### Example 107

### 5-(1-Oxo-1,3-dihydroisobenzofuran-5-yl)-1H-pyrrole-2-carbaldehyde (138) (Scheme 7)

5-Bromoisobenzofuran-1(3H)-one (1.01 g, 4.74 mmol) and 1-(t-butoxycarbonyl)pyrrole-2-boronic acid were reacted according to general method A and partially purified by flash column chromatography on silica gel (10% EtOAc/hexanes as eluant). The crude product, tert-butyl 2-(1-oxo-1,3-dihydroisobenzofuran-5-yl)-1H-pyrrole-1-carboxylate, was obtained as a pale yellow oil (704 mg, 50%) and used directly in the next step. DMF (150 mg, 2.06 mmol) was added to a flask and cooled to 0° C. POCl₃ (316 mg, 2.06 mmol) was added dropwise and the mixture stirred without cooling for 15 min. 1,2-Dichloroethane (1.0 mL) was added and the mixture cooled back to 0° C. A solution of tert-butyl 2-(1-oxo-1,3-dihydroisobenzofuran-5-yl)-1H-pyrrole-1-carboxylate (559 mg, 1.87 mmol) in dichloroethane (1.0 mL) was added dropwise and the resulting bright orange solution heated at reflux for 15 min. Upon cooling to room temperature, NaOAc (845 mg, 10.3 mmol) in water (3.7 mL) was added and heating at reflux continued for a further 15 min. The mixture was allowed to cool, then extracted with CH₂Cl₂ (4×50 mL) and the combined organic fractions washed with sat. NaHCO₃ (100 mL) and dried (Na₂SO₄). Filtration and removal of the solvent under reduced pressure gave a crude product (with the BOC deprotected) which was purified by flash column chromatography on silica gel (5% acetone/CH₂Cl₂ as eluant), followed by trituration with Et₂O. The title compound was isolated as a beige solid (95 mg, 22%), tap (Et₂O) 275-278° C. ¹H NMR [400 MHz, (CD₃)₂SO] δ 12.64 (s, 1H), 9.57 (s, 1H), 8.16 (s, 1H), 8.11 (dd, J=1.8, 1.4 Hz, 1H), 7.87 (d, J=8.1 Hz, 1H), 7.13 (dd, J=3.9, 2.2 Hz, 1H), 6.99 (dd, J=4.0, 2.4 Hz, 1H), 5.45 (s, 2H). LRMS (APCI⁺) calcd for C₁₃H₁₀NO₃ 228 (MH⁺), found 228. Anal. (C₁₃H₉NO₃) C, H, N.

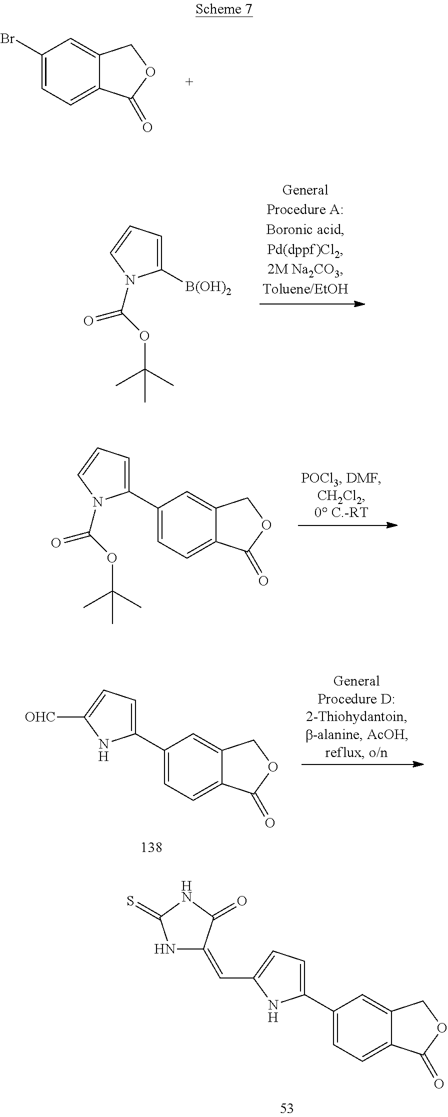 [SMILES: O=C1OCc2cc(Br)ccc21 ; CC(C)(C)OC(=O)n1cccc1B(O)O ; CC(C)(C)OC(=O)n1cccc1-c1ccc2c(c1)COC2=O ; O=Cc1ccc(-c2ccc3c(c2)COC3=O)[nH]1 ; O=C1NC(=S)N/C1=C/c1ccc(-c2ccc3c(c2)COC3=O)[nH]1]

### Example 108

### (E,Z)-5-((5-(1-Oxo-1,3-dihydroisobenzofuran-5-yl)-1H-pyrrol-2-yl)methylene)-2-thioxoimidazolidin-4-one (53) (Scheme 7)

Reaction of 5-(1-oxo-1,3-dihydroisobenzofuran-5-yl)-1H-pyrrole-2-carbaldehyde with 2-thiohydantoin according to general procedure D gave the title compound which was purified by trituration with hot dioxane. The title compound was isolated as a dark red solid (58%), mp (dioxane)>300° C. ¹H NMR [400 MHz, (CD₃)₂SO] δ 13.01 (s, 1H), 12.51 (br s, 1H), 12.19 (br s, 1H), 7.91 (d, J=8.0 Hz, 1H), 7.88 (br s, 1H), 7.85 (dd, J=8.0, 1.3 Hz, 1H), 7.05 (br dd, J=3.8, 2.5 Hz, 1H), 6.99 (br dd, J=3.8, 2.0 Hz, 1H), 6.65 (s, 1H), 5.45 (s, 2H). Anal. (C₁₆H₁₁N₃O₃S.0.25AcOH) C, H, N.

### Example 109

### 1-Oxo-N-(prop-2-ynyl)-1,3-dihydroisobenzofuran-5-carboxamide (139) (Scheme 8)

1-Oxo-1,3-dihydroisobenzofuran-5-carboxylic acid was reacted with, pentafluorophenyl trifluoroacetate, followed by propargylamine according to general procedure F. The crude product was purified by flash column chromatography on silica gel (5% MeOH/CH₂Cl₂ as eluant) give the title compound as white needles (84%), mp (Et₂O) 242-245° C. dec. ¹H NMR [400 MHz, (CD₃)₂SO] δ 9.19 (t, J=5.5 Hz, 1H), 8.10 (s, 1H), 8.01 (dd, J=8.0, 1.4 Hz, 1H), 7.94 (d, J=7.8 Hz, 1H), 5.47 (s, 2H), 4.10 (dd, J=5.5, 2.5 Hz, 2H), 3.14 (t, J=2.5 Hz, 1H). LRMS (APCI⁺) calcd for C₁₂H₁₀NO₃ 216 (MH⁺), found 216. Anal. (C₁₂H₉NO₃) C, H, N.

### Example 110

### 2-(1-Oxo-1,3-dihydroisobenzofuran-5-yl)oxazole-5-carbaldehyde (140) (Scheme 8)

PdCl₂(BnCN)₂ (9 mg, 0.02 mmol) and CuCl₂.2H₂O (8 mg, 0.05 mmol) in DMF were stirred at room temperature for 1 h. A solution of 1-oxo-N-(prop-2-ynyl)-1,3-dihydroisobenzofuran-5-carboxamide (100 mg, 0.47 mmol) was added and the entire mixture stirred at 100° C. under an atmosphere of oxygen for 2 h. Upon cooling, the reaction mixture was partitioned between EtOAc (50 mL) and brine (50 mL). A precipitate formed which was collected by filtration. The EtOAc fraction was dried (Na₂SO₄) and the solvent removed under reduced pressure to give a solid which was combined with the above precipitate. The crude product was purified by flash column chromatography on silica gel (5% MeOH/CH₂Cl₂ as eluant) to give the title compound as a cream solid (18 mg, 17%). ¹H NMR [400 MHz, (CD₃)₂SO] δ 9.87 (s, 1H), 8.42 (s, 2H), 8.28 (dd, J=8.0, 1.4 Hz, 1H), 8.05 (d, J=8.1 Hz, 1H), 5.52 (s, 2H). HRMS (FAB⁺) calcd for C₁₂H₈NO₄ 230.0453 (MH⁺), found 230.0451.

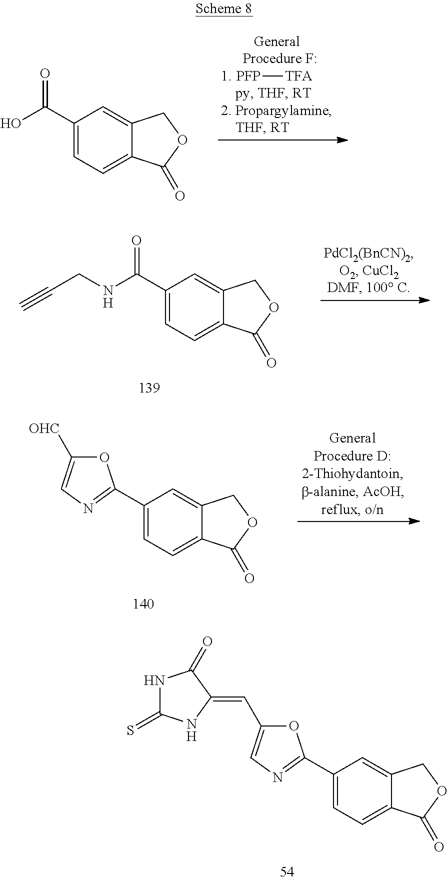

### Example 111

### (E,Z)-5-((2-(1-Oxo-1,3-dihydroisobenzofuran-5-yl)oxazol-5-yl)methylene)-2-thioxoimidazolidin-4-one (54) (Scheme 8)

Reaction of 2-(1-oxo-1,3-dihydroisobenzofuran-5-yl)oxazole-5-carbaldehyde with 2-thiohydantoin according to general procedure D gave the title compound which was purified by trituration with hot dioxane. The title compound was isolated as a bright yellow solid (71%), mp (dioxane)>295° C. ¹H NMR [400 MHz, (CD₃)₂SO] δ 12.54 (br s, 1H), 12.32 (br s, 1H), 8.45-8.51 (m, 2H), 8.06 (dd, J=8.0, 0.4 Hz, 1H), 8.00 (d, J=0.4 Hz; 1H), 6.54 (s, 1H), 5.54 (s, 2 H). LRMS (APCI⁻) calcd for C₁₅H₈N₃O₄S 326 (M-H), found 326. Anal. (C₁₅H₉N₃O₄S) C, H, N.

### Example 112

### 5-(5-(Dimethoxymethyl)thiazol-2-yl)isobenzofuran-1(3H)-one (141) (Scheme 2)

2-Bromo-1,3-thiazole-5-carboxaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired product as a white solid (25%). ¹H NMR [400 MHz, (CD₃)₂SO] δ 8.25 (s, 1H), 8.15 (dt, J=8.0, 0.6 Hz, 1H), 7.98 (d, J=0.7 Hz, 1H), 7.95 (d, J=8.0 Hz, 1H), 5.81 (d, J=0.7 Hz, 1H), 5.48 (s, 2H), 3.35 (s, 6H). LRMS (APCI⁺) calcd for C₁₄H₁₄NO₄S 292 (MH⁺), found 292.

### Example 113

### 2-(1-Oxo-1,3-dihydroisobenzofuran-5-yl)thiazole-5-carbaldehyde (142)

5-((5-(Dimethoxymethyl)thiazol-2-yl)isobenzofuran-1(3H)-one was deprotected according to general method E to give the title compound as a cream solid (100%). ¹H NMR [400 MHz, (CD₃)₂SO] δ 10.13 (s, 1H), 8.86 (s, 1H), 8.40 (d, J=0.7 Hz, 1H), 8.27 (dt, J=8.0, 0.7 Hz, 1H), 8.01 (d, J=8.2 Hz, 1H), 5.51 (s, 2H). HRMS (FAB⁺) calcd for C₁₂H₈NO₃S 246.0225 (MH⁺), found 246.0224.

### Example 114

### (E,Z)-5-((2-(1-Oxo-1,3-dihydroisobenzofuran-5-yl)thiazol-5-yl)methylene)-2-thioxoimidazolidin-4-one (55)

Reaction of 2-(1-oxo-1,3-dihydroisobenzofuran-5-yl)thiazole-5-carbaldehyde with 2-thiohydantoin according to general procedure D gave the title compound as a yellow solid (89%), mp (AcOH)>300° C. ¹H NMR [400 MHz, (CD₃)₂SO] Observe E- and Z- isomers separately. δ 12.14-12.57 (m, 2H), 8.58 (d, J=0.6 Hz, 0.7H), 8.48 (d, J=0.5 Hz, 0.3H), 8.26-8.30 (m, 1H), 8.12-8.19 (m, 1H), 7.95-8.01 (m, 1H), 6.94 (s, 0.3H), 6.82 (s, 0.7H), 5.50 (s, 1.4H), 5.49 (s, 0.6H). LRMS (APCI⁻) calcd for C₁₅H₈N₃O₃S₂ 342 (M-H), found 342. Anal. (C₁₅H₉N₃O₃S₂.0.25H₂O) C, H, N.

### Example 115

### 5-(2-(Dimethoxymethyl)thiazol-4-yl)isobenzofuran-1(3H)-one (143)

4-Bromo-2-formyl-1,3-thiazole was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired product as an off-white solid (16%). ¹H NMR [400 MHz, (CD₃)₂SO] δ 8.27 (d, J=0.6 Hz, 1H), 8.14 (dt, J=8.0, 0.8 Hz, 1H), 7.96 (dd, J=8.0, 0.4 Hz, 1H), 7.84 (d, J=0.7 Hz, 1H), 5.59 (d, J=0.7 Hz, 1H), 5.48 (s, 2H), 3.34 (s, 6H). (APCI⁺) calcd for C₁₄H₁₄NO₄S 292 (MH⁺), found 292.

### Example 116

### 4-(1-Oxo-1,3-dihydroisobenzofuran-5-yl)thiazole-2-carbaldehyde (144)

5-(2-(Dimethoxymethyl)thiazol-4-yl)isobenzofuran-1(3H)-one was deprotected according to general method E to give the title compound as a white solid (100%). ¹H NMR [400 MHz, (CD₃)₂SO] δ 10.02 (s, 1H), 8.88 (s, 1H), 8.35 (s, 1H), 8.23 (d, J=8.0 Hz, 1H), 8.00 (d, J=8.0 Hz, 1H), 5.50 (s, 2H). HRMS (ESI−TOF) calcd for C₁₂H₈NO₃SNa 268.0044 (M+Na), found 268.0039.

### Example 117

### (E,Z)-5-((4-(1-Oxo-1,3-dihydroisobenzofuran-5-yl)thiazol-2-yl)methylene)-2-thioxoimidazolidin-4-one (56)

Reaction of 4-(1-oxo-1,3-dihydroisobenzofuran-5-yl)thiazole-2-carbaldehyde with 2-thiohydantoin according to general procedure D gave the title compound as a yellow solid (73%), mp (AcOH)>295° C. ¹H NMR [400 MHz, (CD₃)₂SO] δ 12.45 (br s, 1H), 11.26 (br s, 1H), 8.43 (s, 1H), 8.38 (dd, J=8.0, 1.4 Hz, 1H), 8.35 (s, 1H), 8.02 (d, J=7.9 Hz, 1H), 6.66 (s, 1H), 5.53 (s, 2H). LRMS (APCI⁻) calcd for C₁₅H₁₅N₃O₃S₂ 342 (M-H), found 342. Anal. (C₁₅H₉N₃O₃S₂) C, H, N.

### Example 118

### 5-(4-(Dimethoxymethyl)thiazol-2-yl)isobenzofuran-1(3H)-one (145)

2-Bromo-1,3-thiazole-4-carboxaldehyde was protected as the dimethyl acetal according to a literature procedure⁵, then reacted with 5-(4,4,5,5-tetramethyl-1,3,2-dioxaborolan-2-yl)isobenzofuran-1(3H)-one according to general procedure A, to give the title compound. Purification by flash column chromatography on silica gel (20% EtOAc/hexanes as eluant) gave the desired product as a pale pink solid (51%). ¹H NMR [400 MHz, (CD₃)₂SO] δ 8.45 (s, 1H), 8.26 (s, 1H), 8.18 (dd, J=8.1, 1.2 Hz, 1H), 7.92 (d, J=8.0 Hz, 1H), 5.72 (s, 1 H), 5.47 (s, 2H), 3.41 (s, 6H). LRMS (APCI⁺) calcd for C₁₄H₁₄NO₄S 292 (MH⁺), found 292.

### Example 119

### 2-(1-Oxo-1,3-dihydroisobenzofuran-5-yl)thiazole-4-carbaldehyde (146)

5-(4-(Dimethoxymethyl)thiazol-2-yl)isobenzofuran-1(3H)-one was deprotected according to general method E to give the title compound as a white solid (100%). ¹H NMR [400 MHz, (CD₃)₂SO] δ 10.05 (d, J=1.2 Hz, 1H), 8.92 (d, J=1.2 Hz, 1H), 8.34-8.36 (m, 1H), 8.26-8.29 (m, 1H), 7.98 (d, J=7.9 Hz, 1H), 5.50 (s, 2H). HRMS (ESI−TOF) calcd for C₁₂H₇NO₃SNa 268.0044 (M+Na), found 268.0039.

### Example 120

### (E,Z)-5-((2-(1-Oxo-1,3-dihydroisobenzofuran-5-yl)thiazol-4-yl)methylene)-2-thioxoimidazolidin-4-one (57)

Reaction of 2-(1-oxo-1,3-dihydroisobenzofuran-5-yl)thiazole-4-carbaldehyde with 2-thiohydantoin according to general procedure D gave the title compound as a yellow-brown solid (88%), mp (AcOH)>295° C. ¹H NMR [400 MHz, (CD₃)₂SO] δ 12.62 (br s, 1H), 11.43 (br s, 1H), 8.83 (s, 1H), 8.54 (s, 1H), 8.37 (dd, J=8.1, 1.2 Hz, 1H), 7.97 (d, J=8.0 Hz, 1H), 6.84 (s, 1H), 5.52 (s, 2H). LRMS (APCI⁺) calcd for C₁₅H₈N₃O₃S₂ 342 (M-H), found 342. Anal. (C₁₅H₉N₃O₃S₂) C, H, N.

### Example 121

### General Procedure G

### 5-(Hydroxymethyl)isobenzofuran-1(3H)-one (147) (Scheme 9)

1-Oxo-1,3-dihydroisobenzofuran-5-carboxylic acid (3.00 g, 16.8 mmol) was dissolved in THF (300 mL), to which, was added carbonyldiimidazole (CDI; 5.46 g, 33.7 mmol). After stirring for 2 h. at room temperature, this reaction mixture was slowly added to a solution of NaBH₄, (3.18 g, 84.0 mmol) in water (80 mL), stirred for a further 5 minutes, then quenched with c. HCl to pH=2. This aqueous mixture was extracted with EtOAc (2×50 mL), the fractions combined, dried (Na₂SO₄) and filtered, then the solvent removed under reduced pressure to afford a crude solid which was purified by flash column chromatography on silica gel (EtOAc as eluant). The title compound was isolated as a white solid (2.59 g, 94%). ¹H NMR in agreement with literature values.¹⁰

### Example 122

### General Procedure H

### 1-Oxo-1,3-dihydroisobenzofuran-5-carbaldehyde (148) (Scheme 9)

Hydroxymethyl)isobenzofuran-1(3H)-one (1.0 g, 6.10 mmol) was dissolved in CH₂Cl₂ (200 mL), pyridinium chlorochromate on silica gel¹¹ (6.71 g of 1 mmol/g silica) added, and the mixture stirred at room temperature for 3 h. All solvent was then removed under reduced pressure and the resulting powder loaded onto a column of flash silica gel which was eluted with CH₂Cl₂ to obtain the title compound as a white solid (620 mg, 63%). ¹H NMR in agreement with literature values.^{10,12}

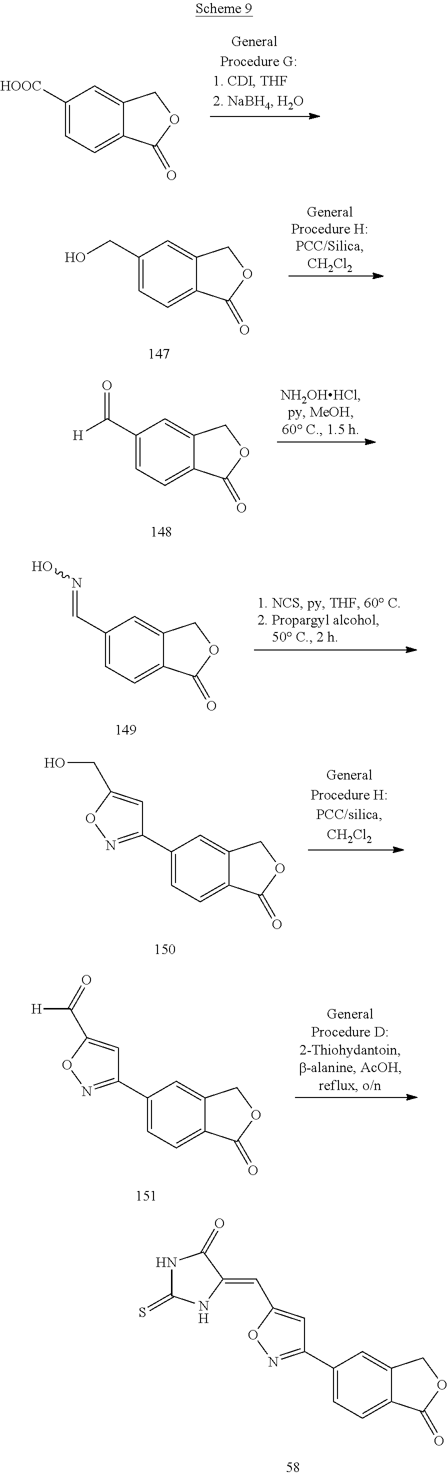

### Example 123

### (E,Z)-1-oxo-1,3-Dihydroisobenzofuran-5-carbaldehyde oxime (149) (Scheme 1)

1-Oxo-1,3-dihydroisobenzofuran-5-carbaldehyde (445 mg, 2.75 mmol) was dissolved in MeOH (40 mL), to which was added pyridine (1.09 g; 13.7 mmol) and NH₂OH.HCl (954 mg, 13.7 mmol). The reaction mixture was stirred at 60° C. for 1.5 h., then upon cooling was diluted with water (100 mL), with the resulting white precipitate collected by filtration and dried to give the title compound (325 mg; 67%). ¹H NMR in agreement with literature values.¹²

### Example 124

### 5-(5-(Hydroxymethyl)isoxazol-3-yl)isobenzofuran-1(3H)-one (150) (Scheme 9)

1-Oxo-1,3-dihydroisobenzofuran-5-carbaldehyde oxime (320 mg, 1.81 mmol) was dissolved in THF (10 mL) then pyridine (1 drop) and N-chlorosuccinimide (266 mg, 1.99 mmol) added. This mixture was heated at 60° C. for 0.5 h. Upon cooling to room temperature, propargyl alcohol (132 mg, 2.35 mmol) and triethylamine (201 mg, 1.99 mmol) were added and a white precipitate formed. The reaction was then heated at 50° C. for 2 h. The solvent was removed under reduced pressure and the residue taken up into CH₂Cl₂ (100 mL), which was then washed with water (2×100 mL), brine (100 mL) and dried (Na₂SO₄). Removal of the solvent afforded a white solid which was purified by flash column chromatography on silica gel (10% acetone/CH₂Cl₂ as eluant) to give the title compound as a fluffy white solid (56%). ¹H NMR [400 MHz, (CD₃)₂SO] δ 8.19 (d, J=0.4 Hz, 1H), 8.04 (dt, J=8.0, 0.7 Hz, 1H), 7.97 (d, J=8.1 Hz, 1H), 7.05 (s, 1H), 5.74 (t, J=6.0 Hz, 1H), 5.49 (s, 2H), 4.65 (d, J=5.7 Hz, 2H). LRMS (APCI⁺) calcd for C₁₂H₁₀NO₄ 232 (MH⁺), found 232.

### Example 125

### 3-(1-Oxo-1,3-dihydroisobenzofuran-5-yl)isoxazole-5-carbaldehyde (151) (Scheme 9)

5-(5-(Hydroxymethyl)isoxazol-3-yl)isobenzofuran-1(3H)-one was oxidized to the corresponding aldehyde according to general procedure H. Following flash column chromatography on silica gel (CH₂Cl₂ as eluant), the title compound was isolated as a white solid (65%). ¹H NMR [400 MHz, (CD₃)₂SO] δ 10.00 (s, 1H), 8.30 (s, 1H), 8.18 (dt, J=8.0, 0.7 Hz, 1H), 8.08 (s, 1H), 8.03 (d, J=8.0 Hz, 1H), 5.52 (s, 2H). LRMS (APCI⁺) calcd for C₁₂H₈NO₄ 230 (MH), found 230.

### Example 126

### (E,Z)-5-((3-(1-Oxo-1,3-dihydroisobenzofuran-5-yl)isoxazol-5-yl)methylene)-2-thioxoimidazolidin-4-one (58) (Scheme 9)

Reaction of 3-(1-oxo-1,3-dihydroisobenzofuran-5-yl)isoxazole-5-carbaldehyde with 2-thiohydantoin according to general procedure D gave the title compound as a yellow solid (86%), mp (AcOH)>300° C. ¹H NMR [400 MHz, (CD₃)₂SO] Observe E- and Z- isomers separately δ 12.65 (br s, 1H), 12.29 (br s, 1H), 8.19 (br s, 0.1H), 8.17 (br s, 0.9H), 8.03-8.09 (m, 2H), 7.95 (s, 0.1H), 7.88 (s, 0.9H), 6.50 (s, 0.1H), 6.44 (s, 0.9H), 5.52 (s, 1.8H), 5.50 (s, 0.2H). LRMS (APCI⁻) calcd for C₁₅H₈N₃O₄S 326 (M-H), found 326. Anal. (C₁₅H₉N₃O₄S.0.5H₂O) C, H, N.

### Example 127

### 5-(1-Oxo-1,3-dihydroisobenzofuran-5-yl)-1,3,4-oxathiazol-2-one (152) (Scheme 10)

1-Oxo-1,3-dihydroisobenzofuran-5-carboxamide (230 mg, 1.30 mmol) was suspended in dioxane (20 mL), chlorocarbonylsulfenyl chloride (340 mg, 2.60 mmol) added, and the mixture heated at reflux for 15 h. Upon cooling, all solvent was removed under reduced pressure and the resulting solid purified by flash column chromatography on silica gel (CH₂Cl₂ as eluant) to give the title compound as a cream solid (244 mg, 80%), mp (CH₂Cl₂/hexane) 195-198° C. ¹H NMR [400 MHz, (CD₃)₂SO] δ 8.24 (dd, J=1.1, 0.7 Hz, 1H), 8.11 (dt, J=8.0, 0.7 Hz, 1H), 8.02 (dd, J=8.1, 0.4 Hz, 1H), 5.50 (s, 2H). LRMS (APCI⁺) calcd for C₁₀H₆NO₄S 236 (MIL), found 236. Anal. (C₁₀H₅NO₄S) C, H, N.

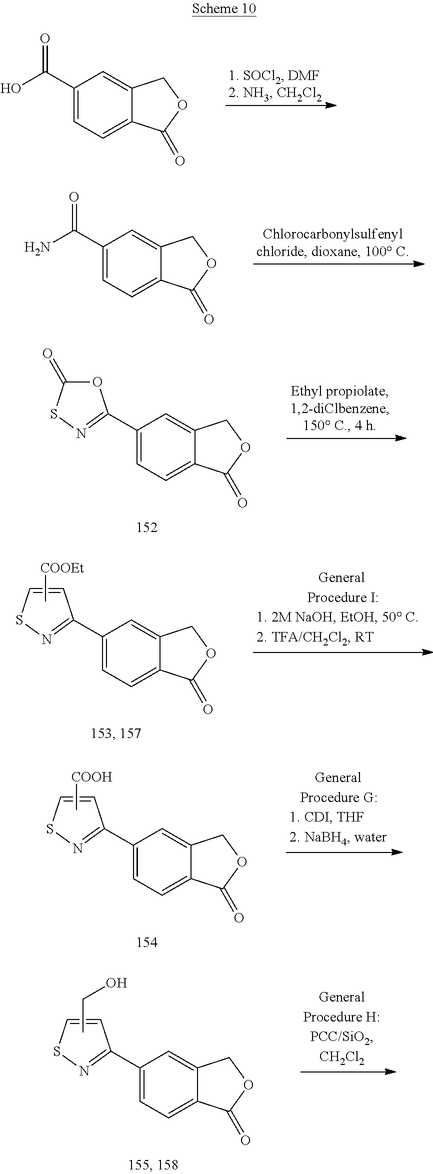

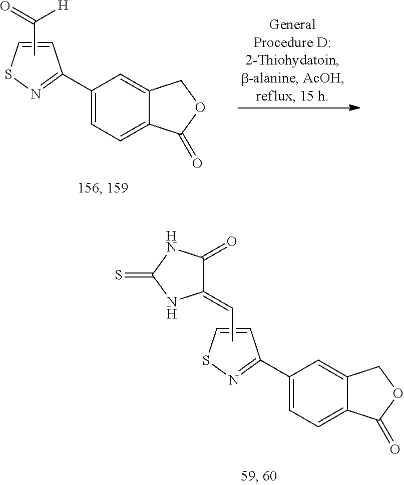

### Example 128

### Ethyl 3-(1-oxo-1,3-dihydroisobenzofuran-5-yl)isothiazole-4-carboxylate (153) (Scheme 10)

A solution of 5-(1-oxo-1,3-dihydroisobenzofuran-5-yl)-1,3,4-oxathiazol-2-one (240 mg, 1.02 mmol) and ethyl propiolate (400 mg, 4.08 mmol) in 1,2-dichlorobenzene (2 mL) was heated at 150° C. for 15 h. All solvent was removed under reduced pressure and the resulting mixture of two isomers (273 mg, 93% total yield) partially separated by flash column chromatography on silica gel (20% EtOAc/hexanes as eluant). The title compound was isolated as a white solid (53 mg, 18%). ¹H NMR [400 MHz, (CD₃)₂SO] δ 9.78 (s, 1H), 7.91 (d, J=8.0 Hz, 1H), 7.88 (br s, 1H), 7.77 (dt, 7.9, 0.6 Hz, 1H), 5.48 (s, 2H), 4.21 (q, J=7.1 Hz, 2H), 1.18 (t, J=7.1 Hz, 3H). LRMS (APCI⁺) calcd for C₁₄H₁₂NO₄S 290 (MO, found 290.

### Example 129

### General Procedure I

### 3-(1-Oxo-1,3-dihydroisobenzofuran-5-yl)isothiazole-4-carboxylic acid (154) (Scheme 10)

Ethyl 3-(1-oxo-1,3-dihydroisobenzofuran-5-yl)isothiazole-4-carboxylate (53 mg, 0.18 mmol) was suspended in EtOH (10 mL), to which was added 2 M NaOH (10 mL). This mixture was stirred at 50° C. for 4 h. Upon cooling, the reaction was diluted with 1 M HCl (25 mL) and extracted with EtOAc (4×25 mL). The combined organic extracts were dried (Na₂SO₄), filtered, and the solvent removed under reduced pressure to afford the intermediate diacid (ring-opened lactone) as a white solid. This solid was dissolved in a mixture of trifluoroacetic acid (2 mL) and CH₂Cl₂ (2 mL) and stirred at R.T. for 4 h. The reaction solvent was removed under reduced pressure to give the title compound as a cream solid (98%). ¹H NMR [400 MHz, (CD₃)₂SO] δ 13.15 (br s, 1H), 9.72 (s, 1H), 7.90 (d, J=8.0 Hz, 1H), 7.87 (s, 1H), 7.77 (dt, J=7.9, 0.7 Hz, 1H), 5.47 (s, 2H). LRMS (APCI⁺) calcd for C₁₂H₈NO₄S 262 (MH⁺), found 262.

### Example 130

### 5-(4-(Hydroxymethyl)isothiazol-3-yl)isobenzofuran-1(3H)-one (155) (Scheme 10)

3-(1-Oxo-1,3-dihydroisobenzofuran-5-yl)isothiazole-4-carboxylic acid was reduced to the corresponding hydroxymethyl compound according to general procedure G. The title compound was isolated as a pale yellow waxy, solid (37 mg, 84%). ¹H NMR [400 MHz, (CD₃)₂SO] δ 9.05 (s, 1H), 8.06 (s, 1H), 8.00 (dt, J=8.0, 0.7 Hz, 1H), 7.95 (d, J=7.9 Hz, 1H), 5.49 (br s, 3H), 4.64 (d, J=0.6 Hz, 2H). LRMS (APCI⁺) calcd for C₁₂H₁₀NO₃S 248 (MH⁺), found 248.

### Example 131

### 3-(1-Oxo-1,3-dihydroisobenzofuran-5-yl)isothiazole-4-carbaldehyde (156) (Scheme 10)

5-(4-(Hydroxymethyl)isothiazol-3-yl)isobenzofuran-1(3H)-one was oxidized to the corresponding aldehyde according to general procedure H. The title compound was isolated as a white solid (84%). ¹H NMR [400 MHz, (CD₃)₂SO] δ 10.01 (s, 1H), 9.98 (s, 1H), 8.00-8.02 (m, 1H), 7.97 (d, J=8.0 Hz, 1H), 7.92 (dt, J=7.9, 0.7 Hz, 1H), 5.50 (s, 2H). LRMS (APCI⁺) calcd for C₁₂H₈NO₃S 246 (MH⁺), found 246.

### Example 132

### (E,Z)-5-((3-(1-Oxo-1,3-dihydroisobenzofuran-5-yl)isothiazol-4-yl)methylene)-2-thioxoimidazolidin-4-one (59) (Scheme 10)

Reaction of, 3-(1-oxo-1,3-dihydroisobenzofuran-5-yl)isothiazole-4-carbaldehyde with 2-thiohydantoin according to general procedure D gave the title compound as a yellow solid (86%), mp (AcOH)>300° C. ¹H NMR [400 MHz, (CD₃)₂SO] δ 12.32 (br s, 2H), 9.70 (d, J=0.50 Hz, 1H), 8.00 (d, J=7.9 Hz, 1H), 7.93 (s, 1H), 7.81 (dt, J=8.0 Hz, 1H), 6.25 (s, 1H), 5.49 (s, 2H). LRMS (APCI⁻) calcd for C₁₅H₈N₃O₃S₂ 342 (M-H), found 342. Anal: (C₁₅H₉N₃O₃S₂) C, H, N.

### Example 133

### Ethyl 3-(1-oxo-1,3-dihydroisobenzofuran-5-yl)isothiazole-5-carboxylate (157) (Scheme 10)

The mixture of two isomers (273 mg, 93% total yield) isolated above was partially separated by flash column chromatography on silica gel (20% EtOAc/hexanes as eluant). The title compound was isolated as a white solid (119 mg, 40%). ¹H NMR [400 MHz, (CD₃)₂SO] δ 8.65 (s, 1H), 8.42 (s, 1H), 8.33 (dt, J=8.1, 0.7 Hz, 1H), 7.96 (d, J=8.1 Hz, 1H), 5.49 (s, 2H), 4.40 (q, J=7.1 Hz, 2H), 1.36 (t, J=7.1 Hz, 3H). LRMS (APCI⁺) calcd for C₁₄H₁₂NO₄S 290 (MH⁺), found 290.

### Example 134

### 5-(5-(Hydroxymethyl)isothiazol-3-yl)isobenzofuran-1(3H)-one (158) (Scheme 10)

Ethyl 3-(1-oxo-1,3-dihydroisobenzofuran-5-yl)isothiazole-5-carboxylate was deprotected under basic conditions to the corresponding carboxylic acid, 3-O-oxo-1,3-dihydroisobenzofuran-5-yl)isothiazole-5-carboxylic acid, according to general procedure I. Without further purification, the crude acid was then reduced to the corresponding hydroxymethyl compound according to general procedure G. The title compound was isolated as a yellow-white solid (49%). ¹H NMR [400 MHz, (CD₃)₂SO] δ 8.28 (s, 1H), 8.20 (dt, J=8.0, 0.7 Hz, 1H), 7.94 (d, J=8.1 Hz, 1H), 7.87 (t, J=1.0 Hz, 1H), 5.97 (t, J=5.6 Hz, 1H), 5.48 (s, 2H), 4.90 (dd, J=5.6, 1.0 Hz, 2H). LRMS (APCI⁺) calcd for C₁₂H₁₀NO₃S 248 (MH⁺), found 248.

### Example 135

### 3-(1-Oxo-1,3-dihydroisobenzofuran-5-yl)isothiazole-5-carbaldehyde (159) (Scheme 10)

5-(5-(Hydroxymethyl)isothiazol-3-yl)isobenzofuran-1(3H)-one was oxidized to the corresponding aldehyde according to general procedure H. The title compound was isolated as a white solid (48%). ¹H NMR [400 MHz, (CD₃)₂SO] δ 10.21 (s, 1H), 8.78 (s, 1H), 8.38 (t, J=0.7 Hz, 1H), 8.29 (dt, J=8.0, 0.7 Hz, 1H), 8.00 (d, J=8.0 Hz, 1H), 5.51 (s, 2H). LRMS (APCI⁺) calcd for C₁₂H₈NO₃S 246 (MH⁺), found 246.

### Example 136

### (E,Z)-5-((3-(1-Oxo-1,3-dihydroisobenzofuran-5-yl)isothiazol-5-yl)methylene)-2-thioxoimidazolidin-4-one (60) (Scheme 10)

Reaction of 3-(1-oxo-1,3-dihydroisobenzofuran-5-yl)isothiazole-5-carbaldehyde with 2-thiohydantoin according to general procedure D gave the title compound as a mustard-yellow solid (79%), mp (AcOH)>300° C. ¹H NMR [400 MHz, (CD₃)₂SO] Observe E- and Z- isomers separately δ 12.59 (br s, 1H), 12.42 (br s, 0.3H), 12.33 (br s, 0.7H), 8.60 (s, 0.7H), 8.42 (s, 0.3H), 8.34 (s, 0.7H), 8.28-8.33 (m, 1H), 8.21 (dd, J=8.1, 1.2 Hz, 0.3H), 8.02 (d, J=8.0 Hz, 0.7H), 7.96 (d, J=8.0 Hz, 0.3H), 6.97 (s, 0.3H), 6.83 (s, 0.7H), 5.52 (s, 1.4H), 5.49 (s, 0.6H). LRMS (APCI⁻) calcd for C₁₅H₈N₃O₃S₂ 342 (M-H), found 342. Anal. (C₁₅H₉N₃O₃S₂) C, H, N.

### Example 137

### General Procedure J

5-(5-(1,3-Dioxolan-2-yl)thiophen-2-yl)isoindolin-1-one (160) (Scheme 11).

2-Thiophenecarboxaldehyde was protected as the cyclic acetal according to a literature procedure¹. 2-(Thiophen-2-yl)-1,3-dioxolane was then reacted with 5-iodoisobenzofuran-1(3H)-one¹³ using a procedure adapted from a literature reference¹⁴. The iodide (1.23 g, 4.75 mmol), PdCl₂(PPh₃)₂ (333 mg, 0.48 mmol) and KF (1.10 g, 19.0 mmol) were weighed into a flask and dissolved in DMSO (35 mL). The mixture was placed under an atmosphere of N₂, the cyclic acetal (2.22 g, 14.2 mmol) and AgNO₃ (807 mg, 4.75 mmol) added, then the resulting suspension stirred for 0.5 h at 100° C. Further portions of AgNO₃ (3×807 mg) were added at 0.5 h. intervals, to give a total reaction time of 2 h. Upon cooling, the mixture was filtered through a plug of celite which was washed well with CHCl₃. The resulting CHCl₃ (ca 200 mL) solution was washed with water (3×100 mL), dried (Na₂SO₄) and the solvent removed under reduced pressure to give a crude solid which was purified by flash column chromatography on silica gel (5% acetone/CH₂Cl₂ as eluant). Trituation with Et₂O gave the desired product as a pale yellow solid (61%). ¹H NMR [400 MHz, (CD₃)₂SO] δ 8.53 (br s, 1H), 7.85 (d, J=0.6 Hz, 1H), 7.76 (dd, J=7.9, 1.6 Hz, 1H), 7.68 (d, J=7.9 Hz, 1H), 7.53 (d, J=3.7 Hz, 1H), 7.25 (d, J=3.6 Hz, 1H), 6.07 (s, 1H), 4.41 (s, 2H), 4.02-4.09 (m, 2H), 3.94-4.01 (m, 2H). LRMS (APCI⁺) calcd for C₁₅H₁₄NO₃S 288 (MH⁺), found 288.

### Example 138

### General Procedure K

### 5-(5-(1,3-Dioxolan-2-yl)thiophen-2-yl)-2-methylisoindolin-1-one (161) (Scheme 11)

5-(5-(1,3-Dioxolan-2-yl)thiophen-2-yl)isoindolin-1-one (150 mg, 0.52 mmol) was dissolved in dry DMF (10 mL) and the resulting solution cooled to 0° C. (ice/water). NaH (23 mg, 0.58 mmol) was added and the mixture stirred for 0.5 h. at this temperature. Methyl iodide (82 mg, 0.58 mmol) was added dropwise to the solution of anion and the reaction stirred for 0.25 h at 0° C., followed by 1 h. at room temperature. The mixture was diluted with water (50 mL) and extracted with EtOAc (3×50 mL). The combined EtOAc fractions were washed with water (3×50 mL), brine (50 mL) and dried (Na₂SO₄). Filtration and removal of the solvent under reduced pressure gave a crude yellow solid which was purified by flash column chromatography on silica gel (5% acetone/CH₂Cl₂ as eluant). The desired product was obtained as a yellow solid (82%). ¹H NMR [400 MHz, (CD₃)₂SO] δ 7.87 (d, J=0.7 Hz, 1H), 7.76 (dd, J=7.9, 1.6 Hz, 1H), 7.67 (d, J=8.0 Hz, 1H), 7.53 (d, J=3.8 Hz, 1H), 7.25 (d, J=3.8 Hz, 1H), 6.07 (s, 1H), 4.49 (s, 2H), 4.02-4.09 (m, 2H), 3.94-3.99 (m, 2H), 3.08 (s, 3H). LRMS (APCI⁺) calcd for C₁₆H₁₆NO₃S 302 (MH⁺), found 302.

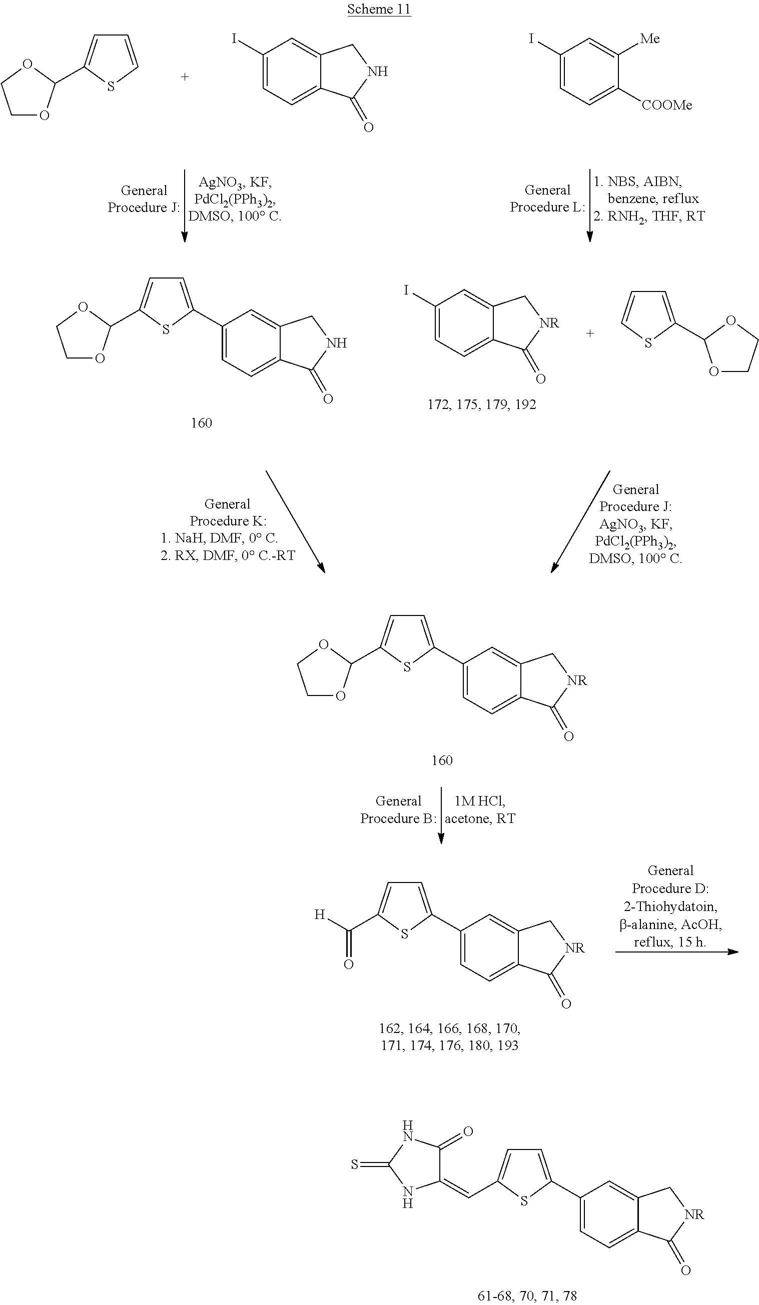

### Example 139

### 5-(2-Methyl-1-oxoisoindolin-5-yl)thiophene-2-carbaldehyde (162) (Scheme 11)

5(5-(1,3-Dioxolan-2-yl)thiophen-2-yl)-2-methylisoindolin-1-one was deprotected according to general procedure B to give the title compound as a yellow solid (89%). ¹H NMR [400 MHz, (CD₃)₂SO]δ 9.94 (s, 1H), 8.08 (d, J=4.0 Hz, 1H), 8.04 (d, J=0.8 Hz, 1H), 7.91 (dd, J=7.9, 1.6 Hz, 1H), 7.86 (d, J=4.0 Hz, 1H), 7.74 (d, J=7.9 Hz, 1H), 4.53 (s, 2H), 3.09 (s, 3H). LRMS (APCI⁺) calcd for C₁₄H₁₂NO₂S 258 (MH⁺), found 258.

### Example 140

### (E,Z)-2-Methyl-5-(5-((5-oxo-2-thioxoimidazolidin-4-ylidene)methyl)thiophen-2-yl)isoindolin-1-one (61) (Scheme 11)

Reaction of 5-(2-methyl-1-oxoisoindolin-5-yl)thiophene-2-carbaldehyde with 2-thiohydantoin according to general procedure D gave the title compound. The desired product was isolated as an orange solid by filtration directly from the reaction mixture (92%), mp (AcOH)>300° C. ¹H NMR [400 MHz, (CD₃)₂SO] Observe E- and Z- isomers separately δ 11.90-12.38 (m, 2H), 7.92 (br d, J=0.7 Hz, 1H), 7.82 (br d, J=4.0 Hz, 0.8H), 7.80-7.84 (m, 1H), 7.75-7.78 (m, 1H), 7.67-7.73 (m, 1.2H), 6.82 (s, 0.2H), 6.65 (s, 0.8H), 4.51 (s, 2H), 3.09 (s, 3H). LRMS (APCI⁻) calcd for C₁₇H₁₂N₃O₂S₂ 354 (M-H), found 354.

### Example 141

### 5-(5-(1,3-Dioxolan-2-yl)thiophen-2-yl)-2-ethylisoindolin-1-one (163)

5-(5-(1,3-Dioxolan-2-yl)thiophen-2-yl)isoindolin-1-one was alkylated with NaH and ethyl iodide according to general procedure K. The desired product was obtained as a pale yellow solid (58%). ¹H NMR [400 MHz, (CD₃)₂SO] δ 7.87 (d, J=0.8 Hz, 1H), 736 (dd, J=7.9, 1.6 Hz, 1H), 7.67 (d, J=7.9 Hz, 1H), 7.53 (d, J=3.7 Hz, 1H), 7.26 (d, J=3.7 Hz, 1H), 6.07 (s, 1H), 4.51 (s, 2H), 4.02-4.09 (m, 2H), 3.94-4.01 (m, 2H), 3.55 (q, J=7.3 Hz, 2H), 1.18 (t, J=7.3 Hz, 3H). LRMS (APCI⁺) calcd for C₁₇H₃NO₃S 316 (MH⁺), found 316.

### Example 142

### 5-(2-Ethyl-1-oxoisoindolin-5-yl)thiophene-2-carbaldehyde (164)

5-(5-(1,3-Dioxolan-2-yl)thiophen-2-yl)-2-ethylisoindolin-1-one was deprotected according to general procedure B to give the title compound as a pale yellow-orange solid (78%). ¹H NMR [400 MHz, (CD₃)₂SO] δ 9.94 (s, 1H), 8.06 (d, J=4.0 Hz, 1H), 8.04 (d, J=0.8 Hz, 1H), 7.92 (dd, J=8.0, 1.6 Hz, 1H), 7.86 (d, J=4.0 Hz, 1H), 7.75 (d, J=7.9 Hz, 1H), 4.55 (s, 2H), 3.56 (q, J=7.3 Hz, 2H), 1.19 (t, J=7.3 Hz, 3H). LRMS (APCI⁺) calcd for C₁₅H₁₄NO₂S 272 (MH⁺), found 272.

### Example 143

### (E,Z)-2-Ethyl-5-(5-((5-oxo-2-thioxoimidazolidin-4-ylidene)methyl)thiophen-2-yl)isoindolin-1-one (62)

Reaction of 5-(2-ethyl-1-oxoisoindolin-5-yl)thiophene-2-carbaldehyde with 2-thiohydantoin according to general procedure D gave the title compound. The desired product was isolated as an orange solid by filtration directly from the reaction mixture (64%), mp (AcOH) 267-270° C. ¹H NMR [400 MHz, (CD₃)₂SO] Observe E- and Z- isomers separately δ 11.98-12.37 (m, 2H), 7.93 (d, J=0.7 Hz, 1H), 7.86 (d, J=4.0 Hz, 0.85H), 7.83 (dd, J=8.0, 1.5 Hz, 1H), 7.75-7.78 (m, 1H), 7.71 (d, J==8.0 Hz, 1H), 7.68 (d, J=4.0 Hz, 0.15H), 6.84 (s, 0.15H), 6.65 (s, 0.85H), 4.53 (s, 2H), 3.56 (q, J=7.2 Hz, 2H), 1.19 (t, J=7.2 Hz, 3H). LRMS (APCI⁻) calcd for C₁₈H₁₄N₃O₂S₂ 368 (M-H), found 368.

### Example 144

### 5-(5-(1,3-Dioxolan-2-yl)thiophen-2-yl)-2-propylisoindolin-1-one (165)

5-(5-(1,3-Dioxolan-2-yl)thiophen-2-yl)isoindolin-1-one was alkylated with NaH and propyl iodide according to general procedure K. The desired product was obtained as a pale yellow solid (45%). ¹H NMR [400 MHz, (CD₃)₂SO] δ 7.87 (d, J=0.7 Hz, 1H), 7.76 (dd, J=8.0, 1.6 Hz, 1H), 7.68 (d, J=8.2 Hz, 1H), 7.53 (d, J=3.7 Hz, 1H), 7.26 (d, J=3.8 Hz, 1H), 6.07 (s, 1H), 4.50 (s, 2H), 4.02-4.09 (m, 2H), 3.94-4.01 (m, 2H), 3.48 (t, J=7.2 Hz, 2H), 1.62 (pentet, J=7.3 Hz, 2H), 0.88 (t, J=7.4 Hz, 3H). LRMS (APCI⁺) calcd for C₁₈H₂₀NO₃S 330 (MH⁺), found 330.

### Example 145

### 5-(1-Oxo-2-propylisoindolin-5-yl)thiophene-2-carbaldehyde (166)

5-(5-(1,3-Dioxolan-2-yl)thiophen-2-yl)-2-propylisoindolin-1-one was deprotected according to general procedure B to give the title compound as a pale yellow solid (65%). ¹H NMR [400 MHz, (CD₃)₂SO] δ 9.94 (s, 1H), 8.08 (d, J=4.0 Hz, 1H), 8.03 (d, J=0.8 Hz, 1H), 7.92 (dd, J=7.9, 1.6 Hz, 1H), 7.86 (d, J=3.9 Hz, 1H), 7.75 (d, J=7.9 Hz, 1H), 4.54 (s, 2H), 3.50 (t, J=7.2 Hz, 2H), 1.63 (pentet, J=7.3 Hz, 2H), 0.88 (t, J=7.4 Hz, 3H). LRMS (APCI⁺) calcd for C₁₆H₁₆NO₂S 286 (MH⁺), found 286.

### Example 146

### (E,Z)-5-(5-((5-Oxo-2-thioxoimidazolidin-4-ylidene)methyl)thiophen-2-yl)-2-propylisoindolin-1-one (63)

Reaction of 5-(1-oxo-2-propylisoindolin-5-yl)thiophene-2-carbaldehyde with 2-thiohydantoin according to general procedure D gave the title compound. The desired product was isolated as an orange solid by filtration directly from the reaction mixture (64%), mp (AcOH) 275-278° C. ¹H NMR [400 MHz, (CD₃)₂SO] Observe E- and Z- isomers separately. δ 11.98-12.38 (m, 2H), 7.92 (br s, 1H), 7.67-7.87 (m, 4H), 6.82 (s, 0.4H), 6.65 (s, 0.6H), 4.52 (s, 2H), 3.48 (t, J=7.2 Hz, 2H), 1.63 (pentet, J=7.3 Hz, 2H), 0.89 (t, J=7.4 Hz, 3H). LRMS (APCI⁻) calcd for C₁₉H₁₆N₃O₂S₂ 382 (M-H), found 382. HRMS (ESI⁻) calcd for C₁₉H₁₆N₃O₂S₂ 382.0689 (M-H), found 382.0697.

### Example 147

### 5-(5-(1,3-Dioxolan-2-yl)thiophen-2-yl)-2-butylisoindolin-1-one (167)

5-(5-(1,3-Dioxolan-2-yl)thiophen-2-yl)isoindolin-1-one was alkylated with NaH and butyl iodide according to general procedure K, except in this case the solution of anion was added to a solution of the butyl iodide in DMF. The desired product was obtained as a pale yellow solid (49%). ¹H NMR [400 MHz, (CD₃)₂SO] δ 7.86 (br d, J=0.7 Hz, 1H), 7.77 (dd, J=8.0, 1.6 Hz, 1H), 7.68 (d, J=8.0 Hz, 1H), 7.53 (d, J=3.9 Hz, 1H), 7.25 (d, J=3.9 Hz, 1H), 6.07 (s, 1H), 4.50 (s, 2H), 4.02-4.09 (m, 2H), 3.94-4.01 (m, 2H), 3.52 (1, J=7.1 Hz, 2H), 1.59 (pentet, J=7.3 Hz, 2H), 1.30 (sextet, J=7.4 Hz, 2H), 0.92 (t, J=7.3 Hz, 3H). LRMS (APCI⁺) calcd for C₁₉H₂₂NO₃S 344 (MH⁺), found 344.

### Example 148

### 5-(2-Butyl-1-oxoisoindolin-5-yl)thiophene-2-carbaldehyde (168)

5-(5-(1,3-Dioxolan-2-yl)thiophen-2-yl)-2-butylisoindolin-1-one was deprotected according to general procedure B to give the title compound as a yellow solid (88%). ¹H NMR [400 MHz, (CD₃)₂SO] δ 9.94 (s, 1H), 8.08 (d, J=4.0 Hz, 1H), 8.03 (br d, J=0.7 Hz, 1H), 7.92 (dd, J=7.9, 1.5 Hz, 1H), 7.86 (d, J=3.9 Hz, 1H), 7.75 (d, J=7.9 Hz, 1H), 4.53 (s, 2H), 3.53 (t, J=7.1 Hz, 2H), 1.60 (pentet, J=7.3 Hz, 2H), 1.30 (sextet, J=7.4 Hz, 2H), 0.92 (t, J=7.3 Hz, 3H), LRMS (APCI⁺) calcd for C₁₇H₁₈NO₂S 300 (MH⁺), found 300.

### Example 149

### (E,Z)-2-Butyl-5-(5-((5-oxo-2-thioxoimidazolidin-4-ylidene)methyl)thiophen-2-yl)isoindolin-1-one (64)

Reaction of 5-(2-butyl-1-oxoisoindolin-5-yl)thiophene-2-carbaldehyde with 2-thiohydantoin according to general procedure D gave the title compound. The desired product was isolated as an orange solid by filtration directly from the reaction mixture (67%), mp (AcOH) 278-281° C. ¹H NMR [400 MHz, (CD₃)₂SO] Observe E- and Z- isomers separately. δ 11.91-12.39 (br m, 2H), 7.92 (br d, J=0.7 Hz, 1H), 7.87 (d, J=4.1 Hz, 0.9H), 7.83 (dd, J=8.0, 1.5 Hz, 1H), 7.77 (d, J=4.0 Hz, 1H), 7.71 (d, J=7.9 Hz, 1H), 7.68 (d, J=4.0 Hz, 0.1H), 6.85 (s, 0.1H), 6.65 (s, 0.9H), 4.52 (s, 2H), 3.53 (t, J=7.1 Hz, 2H), 1.60 (pentet, J=7.3 Hz, 2H), 1.30 (sextet, J=7.4 Hz, 2 H), 0.92 (t, J=7.4 Hz, 3H). LRMS (APCI⁺) calcd for C₂₀H₁₈N₃O₂S₂ 396 (M-H), found 396.

### Example 150

### Ethyl 5-(5-(1,3-dioxolan-2-yl)thiophen-2-yl)-1-oxoisoindoline-2-carboxylate (169)

5-(5-(1,3-Dioxolan-2-yl)thiophen-2-yl)isoindolin-1-one was alkylated with NaH and ethyl chloroformate according to general procedure K. The desired product was obtained as an off-white solid (60%). ¹H NMR [400 MHz, (CD₃)₂SO] δ 7.93 (br d, J=0.7 Hz, 1H), 7.84 (dd, J=8.1, 1.5 Hz, 1H), 7.80 (d, J=8.1 Hz, 1H), 7.59 (d, J=3.9 Hz, 1H), 7.28 (d, J=4.0 Hz, 1H), 6.08 (s, 1H), 4.86 (s, 2H), 4.28 (q, J=7.1 Hz, 2H), 4.02-4.11 (m, 2H), 3.93-4.01 (m, 2H), 1.31 (t, J=7.1 Hz, 3H). LRMS (APCI⁺) calcd for C₁₈H₁₈NO₅S 360 (MH⁺), found 360.

### Example 151

### Ethyl 5-(5-formylthiophen-2-yl)-1-oxoisoindoline-2-carboxylate (170)

Ethyl 5-(5-(1,3-dioxolan-2-yl)thiophen-2-yl)-1-oxoisoindoline-2-carboxylate was deprotected according to general procedure B to give the title compound as a cream solid (100%). ¹H NMR [400 MHz, (CD₃)₂SO] δ 9.96 (s, 1H), 8.08-8.11 (m, 2H), 7.98 (dd, J=8.1, 1.6 Hz, 1H), 7.90 (d, J=4.0 Hz, 1H), 7.87 (d, J=8.2 Hz, 1H), 4.89 (s, 2H), 4.29 (q, J=7.1 Hz, 2H), 1.31 (t, J=7.1 Hz, 3H). LRMS (APCI⁻) calcd for C₁₆H₁₃NO₄S 314 (M-H), found 314.

### Example 152

### (E,Z)-Ethyl 1-oxo-5-(5-((5-oxo-2-thioxoimidazolidin-4-ylidene)methyl)thiophen-2-yl)isoindoline-2-carboxylate (65)

Reaction of ethyl 5-(5-formylthiophen-2-yl)-1-oxoisoindoline-2-carboxylate with 2-thiohydantoin according to general procedure D gave the title compound. The desired product was isolated as an orange solid by filtration directly from the reaction mixture (98%), mp (AcOH) 274-277° C. ¹H NMR [400 MHz, (CD₃)₂SO] Observe E- and Z- isomers separately. δ 11.91-12.39 (m, 2H), 7.97 (br s, 1H), 7.86-7.91 (m, 1.8H), 7.80-7.85 (m, 1.8H), 7.78 (br d, J=4.4 Hz, 0.2H), 7.73 (d, J=4.0 Hz, 0.2H), 6.84 (s, 0.2H), 6.65 (s, 0.8H), 4.88 (s, 2H), 4.28 (q, J=7.1 Hz, 2H), 1.31 (t, J=7.1 Hz, 3H). LRMS (APCI⁻) calcd for C₁₉H₁₄N₃O₄S₂ 412 (M-H), found 412.

### Example 153

### 5-(2-Acetyl-1-oxoisoindolin-5-yl)thiophene-2-carbaldehyde (171)

5-(5-(1,3-Dioxolan-2-yl)thiophen-2-yl)isoindolin-1-one (150 mg, 0.52 mmol) was suspended in acetic anhydride (10 mL), then heated at reflux temperature for 2 h. The solvent was removed under reduced pressure to afford a residue which was dissolved in CH₂Cl₂ (50 mL) and washed with sat. NaHCO₃ (2×50 mL) and brine (50 mL). The organic fraction was dried (Na₂SO₄), filtered and the solvent removed under reduced pressure to give a brown solid. Mass spectral analysis showed a mixture of acetal and aldehyde so this solid was deprotected directly according to general procedure B, then purified by flash column chromatography on silica gel (2% acetone/CH₂Cl₂ as eluant). The desired product was obtained as a pale yellow solid (84%). ¹H NMR [400 MHz, (CD₃)₂SO] δ 9.96 (s, 1H), 8.11-8.13 (br m, 1H), 8.10 (d, J=4.0 Hz, 1H), 8.00 (dd, J=8.1, 1.6 Hz, 1H), 7.89-7.93 (m, 2H), 4.84 (s, 2H), 2.56 (s, 3H). LRMS (APCI⁺) calcd for C₁₅H₁₂NO₃S 286 (MH⁺), found 286.

### Example 154

### (E,Z)-2-Acetyl-5-(5-((5-oxo-2-thioxoimidazolidin-4-ylidene)methyl)thiophen-2-yl)isoindolin-1-one (66)

Reaction of 5-(2-acetyl-1-oxoisoindolin-5-yl)thiophene-2-carbaldehyde with 2-thiohydantoin according to general procedure D gave the title compound. The desired product was isolated as an orange solid by filtration directly from the reaction mixture (79%), mp (AcOH) 336-339° C. ¹H NMR [400 MHz, (CD₃)₂SO] Observe E- and Z- isomers separately. δ 11.91-12.39 (m, 2H), 7.99 (br s, 1H), 7.91 (dd, J=8.1, 1.4 Hz, 1H), 7.85-7.89 (m, 1.85H), 7.83 (d, J=4.0 Hz, 0.85H), 7.78 (br d, J=4.4 Hz, 0.15H), 7.75 (d, J=4.0 Hz, 0.15H), 6.84 (s, 0.15H), 6.65 (s, 0.85H), 4.83 (s, 2H), 2.55 (s, 3H). LRMS (APCI⁺) calcd for C₁₈H₁₂N₃O₃S₂ 382 (M-H), found 382.

### Example 155

### General Procedure L

5-Iodo-2-(2-morpholinoethyl)isoindolin-1-one (172) (Scheme 11). Methyl 4-iodo-2-methylbenzoate¹⁵ (1.00 g, 3.62 mmol) was dissolved in benzene (10 mL), to which was added N-bromosuccinimide (774 mg, 4.35 mmol) and 2,2′-azobis(2-methylpropionitrile) (59 mg, 0.36 mmol). This mixture was heated at reflux temperature for 6 h., the reaction allowed to cool, filtered and the resulting filtrate diluted with Et₂O (100 mL). This solution was washed with sat. sodium metabisufite (50 mL) and brine (50 mL), dried (Na₂SO₄) and filtered. Removal of the solvent under reduced pressure gave a transparent oil which was purified by filtration through a plug of flash silica gel (5% EtOAc as eluant). The resulting oil solidified under high vacuum to afford a white solid (1.17 g), shown to be 93% desired mono-bromide and 7% unreacted starting material. This solid was used directly, in the next step. The above bromide (570 mg, 1.61 mmol) was dissolved in THF (10 mL) and 4-(2-aminoethyl)morpholine (885 mg, 6.80 mmol) added. The reaction mixture was stirred at room temperature for 3 h., then all solvent removed under reduced pressure. The resulting solid was purified by flash column chromatography on silica gel (5% MeOH as eluant). The desired product was obtained as a white solid (61%). ¹H NMR [400 MHz, (CD₃)₂SO] 08.02 (d, J=0.7 Hz, 1H), 7.84 (dd, J=7.9, 1.3 Hz, 1H), 7.45 (d, J=7.9 Hz, 1H), 4.52 (s, 2H), 3.63 (t, J=6.3 Hz, 2H), 3.53 (t, J=4.6 Hz, 4H), 2.53 (t, J=6.4 Hz, 2H), 2.41 (t, J=4.4 Hz, 4H). LRMS (APCI⁺) calcd for C₁₄H₁₅IN₂O₂ 373 (MH⁺), found 373.

### Example 156

### 5-(5-(1,3-Dioxolan-2-yl)thiophen-2-yl)-2-(2-morpholinoethyl)isoindolin-1-one (173)

5-Iodo-2-(2-morpholinoethyl)isoindolin-1-one was reacted with 2-(thiophen-2-yl)-1,3-dioxolane according to general procedure J. The title compound was obtained as a yellow-orange foam (50%). ¹H NMR [400 MHz, (CD₃)₂SO] 0.88 (d, J=0.7 Hz, 1H), 7.76 (dd, J=8.0, 1.5 Hz, 1H), 7.68 (dd, J=7.9, 0.3 Hz, 1H), 7.54 (d, J=3.7 Hz, 1H), 7.26 (dd, J=3.8, 0.4 Hz, 1H), 6.07 (s, 1H), 4.58 (s, 2H), 4.02-4.10 (m, 2H), 3.93-4.01 (m, 2H), 3.65 (t, J=6.3 Hz, 2H), 3.55 (t, J=4.6 Hz, 4H), 2.56 (t, J=6.3 Hz, 2H), 2.39-2.46 (m, 4H). LRMS (APCI⁺) calcd for C₂₁H₂₅N₂O₄S 401 (MH⁺), found 401.

### Example 157

### 5-(2-(2-Morpholinoethyl)-1-oxoisoindolin-5-yl)thiophene-2-carbaldehyde (174)

5-(5-(1,3-Dioxolan-2-yl)thiophen-2-yl)-2-(2-morpholinoethyl)isoindolin-1-one was deprotected according to general procedure B to give the title compound as a yellow solid (81%). ¹H NMR [400 MHz, (CD₃)₂SO] δ 9.94 (s, 1H), 8.08 (d, J=4.0 Hz, 1H), 8.05 (d, J=0.8 Hz, 1H), 7.92 (dd, J=8.0, 1.5 Hz, 1H), 7.86 (d, J=4.0 Hz, 1H), 7.75 (d, J=7.9 Hz, 1H), 4.62 (s, 2H), 3.67 (t, J=6.3 Hz, 2H), 3.55 (t, J=4.6 Hz, 4H), 2.56 (t, J=6.2 Hz, 2H), 2.40-2.47 (m, 4H). LRMS (APCI⁺) calcd for C₁₉H₂₁N₁₂O₃S 357 (MH⁺), found 357.

### Example 158

### (E,Z)-2-(2-Morpholinoethyl)-5-(5-((5-oxo-2-thioxoimidazolidin-4-ylidene)methyl)thiophen-2-yl)isoindolin-1-one (67)

Reaction of 5-(2-(2-morpholinoethyl)-1-oxoisoindolin-5-yl)thiophene-2-carbaldehyde with 2-thiohydantoin according to general procedure D gave the title compound. In this case the AcOH solvent was removed under reduced pressure to afford a orange-black oil which gave an orange solid when suspended in a mixture of acetone (5 mL) and sat. NaHCO₃ (10 mL). This solid was collected by filtration, dried under vacuum and triturated with MeOH to afford the title compound as an orange solid (72%), mp (MeOH) 261-264° C. ¹H NMR [400 MHz, (CD₃)₂SO] Observe E- and Z- isomers separately. δ 11.97-12.29 (m, 2H), 7.94 (s, 1H), 7.80-7.87 (m, 2H), 7.75-7.79 (m, 1H), 7.72 (d, J=8.0 Hz, 0.85H), 7.68 (d, J=3.9 Hz, 0.15H), 6.84 (s, 0.15H), 6.64 (s, 0.15H), 4.61 (s, 2H), 3.66 (t, J=6.2 Hz, 2H), 3.55 (t, J=4.5 Hz, 4H), 2:57 (t, J=6.2 Hz, 2H), 2.40-2.47 (br s, 4H). LRMS (APCI⁺) calcd for C₂₂H₂₃N₄O₃S₂ 455 (MH⁺), found 455.

### Example 159

### 2-(2-Hydroxyethyl)-5-iodoisoindolin-1-one (175)

The title compound was prepared by reaction of methyl 4-iodo-2-methylbenzoate¹⁵ and 2-aminoethanol, according to general procedure L. The desired product was isolated as a crystalline white solid (59%). ¹H NMR [400 MHz, (CD₃)₂SO] δ 8.02 (d, J=0.7 Hz, 1H), 7.84 (dd, J=7.9, 1.4 Hz, 1H), 7.45 (d, J=7.9 Hz, 1H), 4.82 (t, J=5.4 Hz, 1H), 4.52 (s, 2H), 3.58-3.64 (m, 2H), 3.32-3.57 (m, 2H). LRMS (APCI⁺) calcd for C₁₀H₁₁INO₂ 304 (MH⁺), found 304.

### Example 160

### 5-(2-(2-Hydroxyethyl)-1-oxoisoindolin-5-yl)thiophene-2-carbaldehyde (176)

2-(2-Hydroxyethyl)-5-iodoisoindolin-1-one was reacted with 2-(dimethoxymethyl)thiophene according to general procedure J, then deprotected directly to the aldehyde according to general procedure E. The title compound was isolated as a yellow solid (71%). ¹H NMR [400 MHz, (CD₃)₂SO] δ 9.94 (s, 1H), 8.08 (d, J=4.0 Hz, 1H), 8.04 (d, J=0.8 Hz, 1H), 7.92 (dd, J=7.9, 1.6 Hz, 1H), 7.85 (d, J=4.0 Hz, 1H), 7.75 (d, J=8.0 Hz, 1H), 4.83 (t, J=5.4 Hz, 1H), 4.62 (s, 2H), 3.56-3.67 (m, 4H). LRMS (APCI⁺) calcd for C₁₅H₁₄NO₃S 288 (MH⁺), found 288.

### Example 161

### (E,Z)-2-(1-Oxo-5-(5-((5-oxo-2-thioxoimidazolidin-4-ylidene)methyl)thiophen-2-yl)isoindolin-2-yl)ethyl acetate (68)

Reaction of 5-(2-(2-hydroxyethyl)-1-oxoisoindolin-5-yl)thiophene-2-carbaldehyde with 2-thiohydantoin was carried out according to general procedure D to give the title compound as a red powder (57%); mp (AcOH) 261-264° C. ¹H NMR [400 MHz, (CD₃)₂SO] δ 12.39 (s, 1H), 11.98 (s, 1H), 7.96-7.92 (m, 1H), 7.87 (d, J=4.03 Hz, 1H), 7.84 (dd, J=7.98, 1.52 Hz, 1H), 7.77 (d, J=3.98 Hz, 1H), 7.73 (d, J=7.93 Hz, 1H), 6.65 (s, 1H), 4.59 (s, 2H), 4.26 (t, J=5.37, 5.37 Hz, 2H), 3.78 (t, J=5.34, 5.34 Hz, 2H), 2.00 (s, 3H).

### Example 162

### 5-(5-(1,3-Dioxolan-2-yl)thiophen-2-yl)-2,3-dimethylisoindolin-1-one (177)

5-(5-(1,3-Dioxolan-2-yl)thiophen-2-yl)-2-methylisoindolin-1-one was dissolved in dry THF (10 mL) and cooled to 78° C. under N₂. Lithium diisopropylamide in cyclohexane (0.40 mL of a 1.5 M solution, 0.59 mmol) was added dropwise then the mixture stirred for a further 0.25 h. at this temperature. Methyl iodide (84 mg, 0.59 mmol) was added dropwise and stirring continued at −78° C. for 0.5 h., at which point the reaction was allowed to warm to room temperature. The mixture was diluted with sat. NH₄Cl (50 mL) and extracted with EtOAc (3×50 mL). The combined EtOAc fractions were washed with brine (50 mL) and dried (Na₂SO₄). Filtration and removal of the solvent under reduced pressure gave a brown oil which was purified by flash column chromatography on silica gel (5% acetone/CH₂Cl₂ as eluant). The desired product was obtained as a yellow oil (65%). ¹H NMR [400 MHz, (CD₃)₂SO] δ 7.92 (t, J=0.7 Hz, 1H), 7.75 (dd, J=7.9, 1.4 Hz, 1H), 7.66 (d, J=7.9 Hz, 1H), 7.55 (d, J=3.7 Hz, 1H), 7.26 (d, J=3.7 Hz, 1H), 6.07 (s, 1H), 4.59 (q, J=6.7 Hz, 1H), 4.02-4.09 (m, 2H), 3.94-4.02 (m, 2H), 3.01 (s, 3H), 1.47 (d, J=6.7 Hz, 3H). LRMS (APCI⁺) calcd for C₁₇H₁₈NO₃S 316 (MH⁺), found 316.

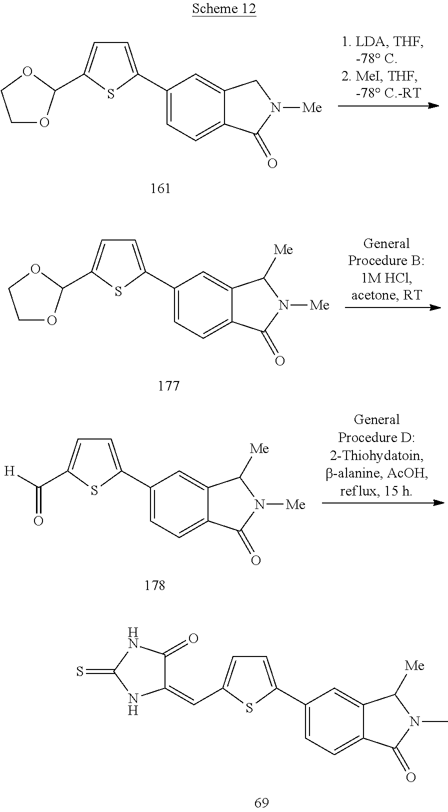 [SMILES: CN1Cc2cc(-c3ccc(C4OCCO4)s3)ccc2C1=O ; CC1c2cc(-c3ccc(C4OCCO4)s3)ccc2C(=O)N1C ; CC1c2cc(-c3ccc(C=O)s3)ccc2C(=O)N1C ; CC1c2cc(-c3ccc(/C=C4/NC(=S)NC4=O)s3)ccc2C(=O)N1C]

### Example 163

### 5-(2,3-Dimethyl-1-oxoisoindolin-5-yl)thiophene-2-carbaldehyde (178)

5-(5-(1,3-Dioxolan-2-yl)thiophen-2-yl)-2,3-dimethylisoindolin-1-one was deprotected according to general procedure B to give the title compound as a beige solid (96%). ¹H NMR [400 MHz, (CD₃)₂SO] δ 9.94 (s, 1H), 8.07-8.10 (m, 2H), 7.91 (dd, J=8.0, 1.4 Hz, 1H), 7.88 (d, J=4.0 Hz, 1H), 7.73 (d, J=7.9 Hz, 1H), 4.63 (q, J=6.7 Hz, 1H), 3.02 (s, 3H), 1.49 (d, J=6.7 Hz, 3H). LRMS (APCI⁺) calcd for C₁₅H₁₄NO₂S 272 (MH⁺), found 272.

### Example 164

### (E,Z)-2,3-Dimethyl-5-(5-((5-oxo-2-thioxoimidazolidin-4-ylidene)methyl)thiophen-2-yl)isoindolin-1-one (69)

Reaction of 5-(2,3-dimethyl-1-oxoisoindolin-5-yl)thiophene-2-carbaldehyde with 2-thiohydantoin according to general procedure D gave the title compound. The desired product was isolated as an orange solid by filtration directly from the reaction mixture (57%), mp (AcOH) 286° C. (dec.). ¹H NMR [400 MHz, (CD₃)₂SO] Observe E- and Z- isomers separately. δ 11.92-12.38 (m, 2H), 7.95-7.99 (m, 1H), 7.87 (d, J=4.0 Hz, 0.75H), 7.72-7.82 (m, 2H), 7.68-7.72 (m, 1.25H), 6.84 (s, 0.25H), 6.64 (s, 0.75H), 4.61 (q, J=6.7 Hz, 1H), 3.02 (s, 3H), 1.48 (d, J=6.7 Hz, 3H). HRMS (ESI⁻) calcd for C₁₈H₁₄N₃O₂S₂ 368.0533 (M-H), found 368.0526.

### Example 165

### (E,Z)-2-(2-Hydroxyethyl)-5-(5-((5-oxo-2-thioxoimidazolidin-4-ylidene)methyl)thiophen-2-yl)isoindolin-1-one (70) (scheme 11)

Hydrolysis of (E,Z)-2-(1-oxo-5-(5-((5-oxo-2-thioxoimidazolidin-4-ylidene)methyl)thiophen-2-yl)isoindolin-2-yl)ethyl acetate (68) to the alcohol was carried out by treating the crude product with K₂CO₃ (5 equivalents) in a mixture of MeOH/water (5:1). The desired product was isolated as an orange solid by filtration directly from the reaction mixture (48%), mp (AcOH) 285° C. (dec.). ¹H NMR [400 MHz, (CD₃)₂SO] Observe E- and Z- isomers separately. δ 11.98-12.39 (m, 2H), 7.93 (br s, 1H), 7.87 (d, J=4.0 Hz, 1H), 7.83 (dd, J=8.0, 1.5 Hz, 0.9H), 7.77 (d, J=4.0 Hz, 1H), 7.72 (d, J=8.0 Hz, 1H), 7.68 (d, J=4.0 Hz, 0.1H), 6.84 (s, 0.1H), 6.65 (s, 0.9H), 4.82 (t, J=5.2 Hz, 1H), 4.60 (s, 2H), 3.62-3.67 (m, 2H), 3.56-3.61 (m, 2H). LRMS (APCI⁻) calcd for C₁₈H₁N₃O₃S₂ 384 (M-H), found 384.

### Example 166

### 2-(3-Hydroxypropyl)-5-iodoisoindolin-1-one (179) (scheme 11)

The title compound was prepared by reaction of methyl 4-iodo-2-methylbenzoate¹⁵ and 3-amino-1-propanol, according to general procedure L. The desired product was isolated as a crystalline white solid (65%). ¹H NMR [400 MHz, (CD₃)₂SO] δ 8.02 (d, J=0.7 Hz, 1H), 7.84 (dd, J=7.9, 1.4 Hz, 1H), 7.45 (d, J=7.9 Hz, 1H), 4.48 (t, J=5.1 Hz, 1H), 4.45 (s, 2H), 3.55 (t, J=7.2 Hz, 2H), 3.43 (q, J=5.9 Hz, 2H), 1.70-1.77 (m, 2H). LRMS (APCI⁺) calcd for C₁₁H₁₃INO₂ 318 (MH), found 318.

### Example 167

### 5-(2-(3-Hydroxypropyl)-1-oxoisoindolin-5-yl)thiophene-2-carbaldehyde (180)

2-(3-Hydroxypropyl)-5-iodoisoindolin-1-one was reacted with 2-(dimethoxymethyl)thiophene according to general procedure J, then deprotected directly to the aldehyde according to general procedure E. The title compound was isolated as a yellow solid (50%). ¹H NMR [400 MHz, (CD₃)₂SO] δ 9.94 (s, 1H), 8.08 (d, J=4.0 Hz, 1H), 8.03 (d, J=0.8 Hz, 1H), 7.92 (dd, J=7.9, 1.6 Hz, 1H), 7.86 (d, J=4.0 Hz, 1H), 7.75 (d, J=8.0 Hz, 1H), 4.55 (s, 2H), 4.50 (t, J=5.2 Hz, 1H), 3.58 (q, J=7.5 Hz, 2H), 3.46 (q, J=5.8 Hz, 2H), 1.76 (pentet, J=6.3 Hz, 2H). LRMS (APCI⁺) calcd for C₁₆H₁₆NO₃S 302 (MH⁺), found 302.

### Example 168

### (E,Z)-2-(3-Hydroxypropyl)-5-(5-((5-oxo-2-thioxoimidazolidin-4-ylidene)methyl)thiophen-2-yl)isoindolin-1-one (71)

Reaction of 5-(2-(3-hydroxypropyl)-1-oxoisoindolin-5-yl)thiophene-2-carbaldehyde with 2-thiohydantoin was carried out according to general procedure D gave the title compound as the O-acetate.

Hydrolysis of the acetate to the desired alcohol was carried out by treating the crude product with K₂CO₃ (5 equivalents) in a mixture of MeOH/water (5:1). The desired product was isolated as an orange solid by filtration directly from the reaction mixture (64%), mp (MeOH) 240-243° C. ¹H NMR [400 MHz, (CD₃)₂SO] Observe E- and Z-isomers separately. δ 11.98-12.40 (m, 2H), 7.93 (br s, 1H), 7.87 (d, J=4.0 Hz, 0.9H), 7.83 (dd, J=8.0, 1.5 Hz, 1H), 7.77 (d, J=4.0 Hz, 1H), 7.71 (d, J=8.1 Hz, 1H), 7.68 (d, J=4.0 Hz, 0.1H), 6.84 (s, 0.1H), 6.65 (s, 0.9H), 4.54 (s, 2H), 4.50 (t, J=5.1 Hz, 1H), 3.58 (t, J=7.2 Hz, 2H), 3.46 (q, J=5.8 Hz, 2H), 1.77 (pentet, J=6.7 Hz, 2H). LRMS (APCI⁺) calcd for C₁₉H₁₃N₃O₃S₂ 400 (MK), found 400.

### Example 169

### 5-(3-Oxoisoindolin-5-yl)thiophene-2-carbaldehyde (181) (scheme 13)

6-(4,4,5,5-Tetramethyl-1,3,2-dioxaborolan-2-yl)isoindolin-1-one was reacted with 2-(5-bromothiophen-2-yl)-1,3-dioxolane according to general procedure A, then deprotected directly to the aldehyde according to general procedure B. The title compound was isolated as an off-white solid (84%). ¹H NMR [400 MHz, (CD₃)₂SO] δ 9.93 (s, 1H), 8.68 (br s, 1H), 8.07 (d, J=3.9 Hz, 1H), 8.00-8.04 (m, 2H), 7.87 (d, J=3.9 Hz, 1H), 7.70 (br d, J=, 8.5 Hz, 1H), 4.44 (s, 2H). LRMS (APCI⁺) calcd for C₁₃H₁₀NO₂S 244 (MH⁺), found 244.

### Example 170

### (E,Z)-6-(5-((5-Oxo-2-thioxoimidazolidin-4-ylidene)methyl)thiophen-2-yl)isoindolin-1-one (72)

Reaction of 5-(3-oxoisoindolin-5-yl)thiophene-2-carbaldehyde with 2-thiohydantoin was carried out according to general procedure D gave the title compound as an orange solid (60%), mp (DMSO/water)>320° C. ¹H NMR [400 MHz, (CD₃)₂SO] Observe E- and Z- isomers separately. δ 11.97-12.37 (m, 2H), 8.63 (br s, 1H), 7.93-7.97 (m, 2H), 7.83 (br d, J=4.0 Hz, 1H), 7.76 (br d, J=4.0 Hz, 1H), 7.75 (d, J=8.5 Hz, 1H), 6.84 (s, 0.05H), 6.66 (s, 0.95H), 4.42 (s, 2H). LRMS (APCI⁻) calcd for C₁₆H₁₀N₃O₃S₂ 340 (M-H), found 340.

### Example 171

### 6-Bromo-2-methylisoindolin-1-one (182)

6-Bromoisoindolin-1-one was alkylated with methyl idodide and NaH according to general procedure K. The title compound was isolated as a pale yellow solid (68%). ¹H NMR [400 MHz, (CD₃)₂SO] δ 7.74-7.79 (m, 2H), 7.56 (dd. J=7.9, 0.7 Hz, 1H), 4.44 (s, 2H), 3.07 (s, 3H). LRMS (APCI⁺) calcd for C₉H₉BrNO 226, 228 (MH⁺), found 226, 228.

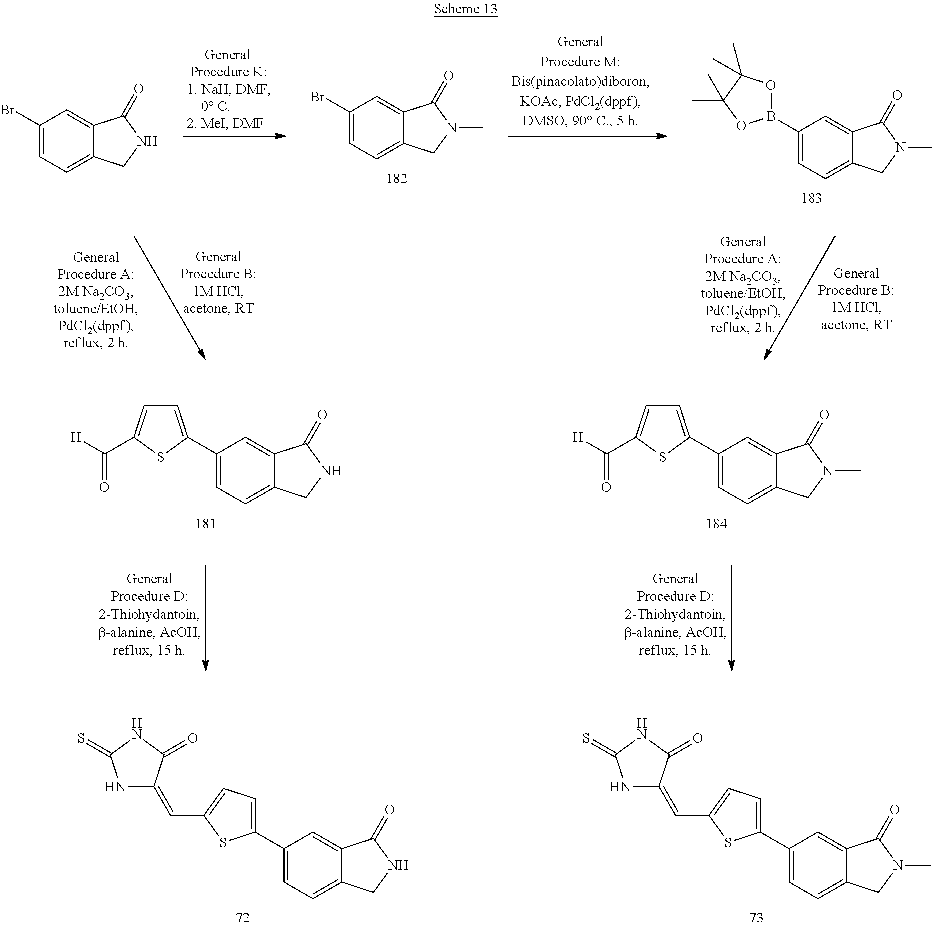

### Example 172

### General Procedure M

### 2-Methyl-6-(4,4,5,5-tetramethyl-1,3,2-dioxaborolan-2-yl)isoindolin-1-one (183)

6-Bromo-2-methylisoindolin-1-one (723 mg, 3.41 mmol), bis(pinacolato)diboron (1.04 g, 4.09 mmol), potassium acetate (1.00 g, 10.2 mmol) and PdCl₂(dppf) catalyst (139 mg, 0.17 mmol) were weighed into a flask which was sealed under N₂. DMSO (15 mL) was added, and the entire mixture stirred at 90° C. for 5 h. Upon cooling, the reaction mixture was diluted with water (250 mL) and extracted with CH₂Cl₂ (5×50 mL). The combined CH₂Cl₂ fractions were in turn washed with water (2×100 mL), brine (100 mL), dried (Na₂SO₄), filtered and the solvent removed under reduced pressure to yield the crude product. Purification was carried out by flash column chromatography on silica gel (20% THF/CH₂Cl₂ as eluant) to give the title compound as a crystalline beige solid (262 mg, 28%). ¹H NMR [400 MHz, (CD₃)₂SO] δ 7.91 (br s, 1H), 7.85 (dd, J=7.6, 1.0 Hz, 1H), 7.59 (dd, J=7.5, 0.6 Hz, 1H), 4.49 (s, 2H), 3.07 (s, 3H), 1.32 (s, 12H). LRMS (APCI⁺) calcd for C₁₅H₂₁BNO₃ 274 (MH⁺), found 274.

### Example 173

### 5-(2-Methyl-3-oxoisoindolin-5-yl)thiophene-2-carbaldehyde (184)

2-Methyl-6-(4,4,5,5-tetramethyl-1,3,2-dioxaborolan-2-yl)isoindolin-1-one was reacted with 2-(5-bromothiophen-2-yl)-1,3-dioxolane according to general procedure A, then deprotected directly to the aldehyde according to general procedure B. The title compound was isolated as a pale yellow solid (91%). ¹H NMR [400 MHz, (CD₃)₂SO] δ 9.93 (s, 1H), 8.06 (d, J=4.0 Hz, 1H), 7.99-8.03 (m, 2H), 7.88 (d, J=4.0 Hz, 1H), 7.70 (dd, J=7.8, 0.8 Hz, 1H), 4.52 (s, 2H), 3.10 (s, 3H). LRMS (APCI⁺) calcd for C₁₄H₁₂NO₂S 258 (MH⁺), found 258.

### Example 174

### (E,Z)-2-Methyl-6-(5-((5-oxo-2-thioxoimidazolidin-4-ylidene)methyl)thiophen-2-yl)isoindolin-1-one (73)

Reaction of 5-(2-methyl-3-oxoisoindolin-5-yl)thiophene-2-carbaldehyde with 2-thiohydantoin was carried out according to general procedure D gave the title compound as an orange solid (89%), mp (AcOH)>310° C. ¹H NMR [400 MHz, (CD₃)₂SO] Observe E- and Z- isomers separately. δ 11.97-12.34 (m, 2H), 7.91-7.96 (m, 2H), 7.82 (d, J=4.0 Hz, 0.85H), 7.75-7.78 (m, 1H), 7.69 (d, J=4.0 Hz, 0.15H), 7.66 (d, J=8.6 Hz, 1H), 6.84 (s, 0.15H), 6.66 (s, 0.85H), 4.40 (s, 2H), 3.10 (s, 3H). LRMS (APCI⁻) calcd for C₁₇H₁₂N₃O₃S₂ 354 (M-H), found 354.

### Example 175

### General Procedure N

### 5-(5-(Dimethoxymethyl)thiophen-2-yl)-2-(3-hydroxypropyl)isoindolin-1-one (185) (scheme 14)

5-(2-(3-Hydroxypropyl)-1-oxoisoindolin-5-yl)thiophene-2-carbaldehyde (180) (1.20 g, 3.98 mmol) was dissolved in a mixture of MeOH (50 mL) and trimethylorthoformate (5 mL). p-Toluenesulfonic acid (100 mg) was added, along with 4 A molecular sieves (5.0 g), then the reaction mixture heated at reflux for 15 h. Upon cooling, the reaction mixture was diluted with CH₂Cl₂ (100 mL) and filtered through celite. The solvent was removed from the combined filtrate and washings under reduced pressure to give an oil which was dissolved in EtOAc (150 mL) and washed with sat. NaHCO₃ (2×100 mL), water (100 mL) and brine (100 mL). The organic phase was dried (Na₂SO₄), filtered and the solvent removed to afford the title compound as a waxy, pale yellow solid (100%). ¹H NMR [400 MHz, (CD₃)₂SO]δ 7.86 (d, J=0.8 Hz, 1H), 7.75 (dd, J=8.0, 1.5 Hz, 1H), 7.67 (d, J=7.9 Hz, 1H), 7.54 (d, J=3.7 Hz, 1H), 7.02 (dd, J=3.8, 0.8 Hz, 1H), 5.66 (d, J=0.7 Hz, 1H), 4.51 (s, 2H), 4.49 (t, J=5.1 Hz, 1H), 3.57 (t, J=7.2 Hz, 2H), 3.45 (q, J=5.9 Hz, 2H), 3.32 (s, 6H), 1.76 (pentet, J=6.7 Hz, 2H). LRMS (APCI⁺) calcd for C₁₈H₂₂NO₄S 348 (MH⁺), found 348.

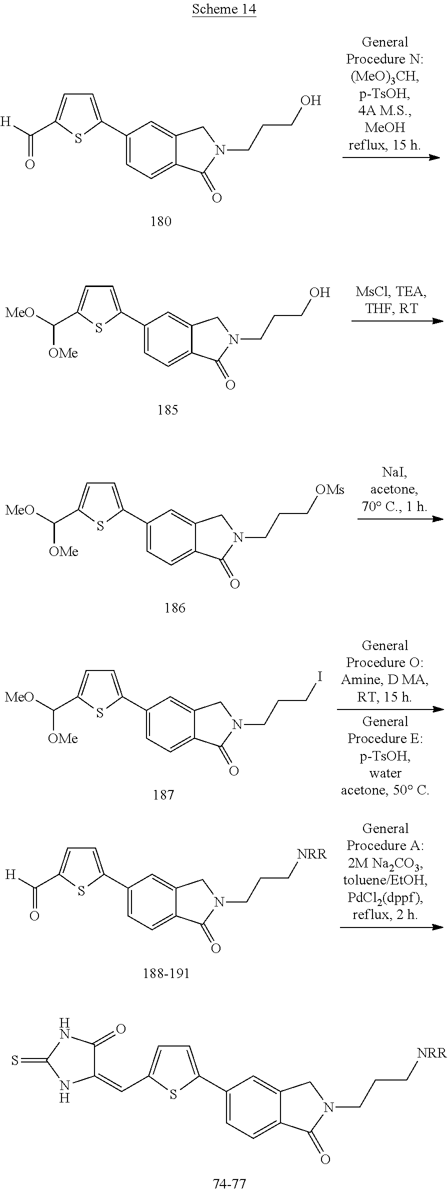

### Example 176

### 3-(5-(5-(Dimethoxymethyl)thiophen-2-yl)-1-oxoisoindolin-2-yl)propyl methanesulfonate (186)

5-(5-(Dimethoxymethyl)thiophen-2-yl)-2-(3-hydroxypropyl)isoindolin-1-one (1.29 g, 3.72 mmol) was dissolved in dry THF (40 mL). Triethylamine (3.76 g, 37.2 mmol) and methanesulfonyl chloride (1.70 g, 14.9 mmol) were added and the reaction mixture stirred at room temperature for 3.5 h. The mixture was diluted with EtOAc (200 mL), which was washed with water (100 mL), sat. NaHCO₃ (100 mL) and brine (100 mL). The organic layer was dried (Na₂SO₄), filtered, and the solvent removed under reduced pressure to give a residue which was purified by flash column chromatography on silica gel (5% acetone/CH₂Cl₂ as eluant). The title compound was obtained as a waxy yellow solid (1.58 g, 100%). ¹H NMR [400 MHz, (CD₃)₂SO] δ 7.87 (d, J=0.7 Hz, 1H), 7.76 (dd, J=7.9, 1.5 Hz, 1H), 7.69 (d, J=7.6 Hz, 1H), 7.55 (d, J=3.7 Hz, 1H), 7.12 (dd, J=3.7, 0.8 Hz, 1H), 5.66 (d, J=0.6 Hz, 1H), 4.53 (s, 2H), 4.25 (t, J=6.2 Hz, 2H), 3.63 (t, J=6.9 Hz, 2H), 3.32 (s, 6H), 3.18 (s, 3H), 2.04 (pentet, J=6.6 Hz, 2H). LRMS (APCI⁺) calcd for C₁₉H₂₄NO₆S₂ 426 (MH⁺), found 426.

### Example 177

### 5-(5-(Dimethoxymethyl)thiophen-2-yl)-2-(3-iodopropyl)isoindolin-1-one (187)

3-(5-(5-(Dimethoxymethyl)thiophen-2-yl)-1-oxoisoindolin-2-yl)propyl methanesulfonate (624 mg, 1.47 mmol) was dissolved in acetone (30 mL) at 70° C., then NaI (4.40 g, 29.3 mmol) added. Stirring was continued at this temperature for 1 h. then the mixture allowed to cool and filtered through celite. The solvent was removed from the combined filtrate and washings under reduced pressure to give a residue which was purified by flash column chromatography on silica gel (10% acetone/CH₂Cl₂ as eluant). The title compound was isolated as a waxy yellow solid (660 mg, 96%). ¹H NMR [400 MHz, (CD₃)₂SO] δ 7.86 (d, J=0.8 Hz, 1H), 7.76 (dd, J=7.9, 1.6 Hz, 1H), 7.68 (d, J=7.9 Hz, 1H), 7.55 (d, J=3.7 Hz, 1H), 7.12 (dd, J=3.7, 0.8 Hz, 1H), 5.66 (d, J=0.7 Hz, 1H), 4.53 (s, 2H), 3.58 (t, J=6.9 Hz, 2H), 3.32 (s, 6H), 3.25 (t, J=6.9 Hz, 2H), 2.14 (pentet, J=6.9 Hz, 2H). LRMS (APCI⁺) calcd for C₁₈H₂₁INO₃S 458 (MH⁺), found 458.

### Example 178

### General Procedure O

### 5-(2-(3-(Dimethylamino)propyl)-1-oxoisoindolin-5-yl)thiophene-2-carbaldehyde (188)

5-(5-(Dimethoxymethyl)thiophen-2-yl)-2-(3-iodopropyl)isoindolin-1-one (330 mg, 0.71 mmol) was dissolved in dimethylacetamide (10 mL), to which was added dimethylamine (3.53 mL of a 2 M solution in THF) and the resulting mixture stirred for 15 h. at room temperature. All solvent was removed under reduced pressure to give a viscous oil which was dissolved in EtOAc (100 mL). This solution was washed with water (3×100 mL), brine (100 mL) and dried (Na₂SO₄). Filtration and removal of the solvent under reduced pressure gave the crude dimethyl acetal-protected product as a yellow oil. This product was then deprotected directly to the desired aldehyde according to general procedure E. Trituration with Et₂O gave the title compound as a yellow solid (140 mg, 60%). ¹H NMR [400 MHz, (CD₃)₂SO] δ 9.94 (s, 1H), 8.08 (d, J=4.0 Hz, 1H), 8.03 (d, J=0.7 Hz, 1H), 7.92 (dd, J=8.0, 1.6 Hz, 1H), 7.86 (d, J=4.0 Hz, 0.1H), 7.75 (d, J=7.9 Hz, 1H), 4.55 (s, 2H), 3.56 (t, J=7.2 Hz, 2H), 2.24 (t, J=7.1 Hz, 2H), 2.13 (s, 6H), 1.74 (pentet, J=7.2 Hz, 2H). LRMS (APCI⁺) calcd for C₁₈H₂₁N₂O₂S 329 (MH⁺), found 329.

### Example 179

### (E)-2-(3-(Dimethylamino)propyl)-5-(5-((5-oxo-2-thioxoimidazolidin-4-ylidene)methyl)thiophen-2-yl)isoindolin-1-one (74)

Reaction of 5-(2-(3-(dimethylamino)propyl)-1-oxoisoindolin-5-yl)thiophene-2-carbaldehyde with 2-thiohydantoin was carried out according to general procedure D. In this case the reaction solvent was removed under reduced pressure and the residue suspended in acetone (5 mL), to which was added sat. NaHCO₃ (5 mL). This mixture was stirred at room temperature for 1 h., the solid collected by filtration, dried, and re-suspended in MeOH (5 mL). After stirring for 10 min. at room temperature, the title compound was obtained by filtration as a dark orange solid (51%), mp (MeOH) 212° C. (dec.). ¹H NMR [400 MHz, (CD₃)₂SO] δ 11.47 (v br s, 2H), 7.91 (br s, 1H), 7.82 (dd, J=8.0, 1.4 Hz, 1H), 7.67-7.73 (m, 3H), 6.52 (s, 1H), 4.53 (s, 2H), 3.56 (t, J=7.0 Hz, 2H), 2.47 (m, 2H), 2.33 (s, 6H), 1.81 (pentet, J=7.2 Hz, 2H). LRMS (APCI⁺) calcd for C₂₁H₂₃N₄O₂S₂ 427 (MH⁺), found 427.

### Example 180

### 5-(1-Oxo-2-(3-(piperidin-1-yl)propyl)isoindolin-5-yl)thiophene-2-carbaldehyde (189)

5-(5-(Dimethoxymethyl)thiophen-2-yl)-2-(3-iodopropyl)isoindolin-1-one was reacted with piperidine according to general procedure 0, followed by deprotection according to general procedure E to give the title compound as a yellow solid (73%). ¹H NMR [400 MHz, (CD₃)₂SO] 9.94 (s, 1H), 8.08 (d, J=4.0 Hz, 1H), 8.03 (br s, 1H), 7.92 (dd, J=8.0, 1.5 Hz, 1H), 7.86 (d, J=4.0 Hz, 1H), 7.74 (d, J=8.0 Hz, 1H), 4.55 (s, 2H), 3.56 (t, J=−7.2 Hz, 2H), 2.22-2.34 (m, 6H), 1.77 (pentet, J=7.0 Hz, 2H), 1.45 (pentet, J=5.3 Hz, 4H), 1.31-1.38 (m, 2H). LRMS (APCI⁺) calcd for C₂₁H₂₅N₂O₂S 369 (MH⁺), found 369.

### Example 181

### (E,Z)-5-(5-((5-Oxo-2-thioxoimidazolidin-4-ylidene)methyl)thiophen-2-yl)-2-(3-(piperidin-1-yl)propyl)isoindolin-1-one (75)

Reaction of 5-(1-oxo-2-(3-(piperidin-1-yl)propyl)isoindolin-5-yl)thiophene-2-carbaldehyde with 2-thiohydantoin was carried out according to general procedure D. The reaction solvent was removed under reduced pressure and the residue suspended in acetone (5 mL), to which was added sat. NaHCO₃ (5 mL). This mixture was stirred at room temperature for 1 h., the solid collected by filtration, dried, and re-suspended in MeOH (5 mL). After stirring for 10 min. at room temperature, the title compound was obtained by filtration as an orange solid (48%), mp (dioxane) 221-224° C. ¹H NMR [400 MHz, (CD₃)₂SO] Observe E- and Z- isomers separately. δ 11.47 (v br s, 2H), 7.91 (br s, 1H), 7.82 (dd, J=8.0, 1.4 Hz, 1H), 7.77 (d, J=4.4 Hz, 0.15H), 7.67-7.72 (m, 2.85H), 6.84 (s, 0.15H), 6.52 (s, 0.85H), 4.53 (s, 2H), 3.51 (t, J=6.9 Hz, 2H), 2.44-2.57 (m, 6H), 1.83 (pentet, J=7.1 Hz, 0.2H), 1.53 (pentet, J=5.4 Hz, 4H), 1.35-1.43 (m, 2H). HRMS (ESI⁺) calcd for C₂₄H₂₇N₄O₂S₂ 467.1570 (MH⁺), found 467.1567.

### Example 182

### 5-(2-(3-(4-Methylpiperazin-1-yl)propyl)-1-oxoisoindolin-5-yl)thiophene-2-carbaldehyde (190)

5-(5-(Dimethoxymethyl)thiophen-2-yl)-2-(3-iodopropyl)isoindolin-1-one was reacted with 1-methylpiperazine according to general procedure O, followed by deprotection according to general procedure E to give the title compound as a yellow solid (78%). ¹H NMR [400 MHz, (CD₃)₂SO] δ 9.94 (s, 1H), 8.08 (d, J=4.0 Hz, 1H), 8.03 (d, J=0.7 Hz, 1H), 7.92 (dd, J=8.0, 1.6 Hz, 1H), 7.86 (d, J=3.9 Hz, 1H), 7.74 (d, J=8.0 Hz, 1H), 4.54 (s, 2H), 3.55 (t, J=7.1 Hz, 2H), 2.20-2.38 (m, 10H), 2.11 (s, 3H), 1.76 (pentet, J=7.0 Hz, 2H). LRMS (APCI⁺) calcd for C₂₁H₂₆N₃O₂S 384 (MH⁺), found 384.

### Example 183

### (E,Z)-2-(3-(4-Methylpiperazin-1-yl)propyl)-5-(5-((5-oxo-2-thioxoimidazolidin-4-ylidene)methyl)thiophen-2-yl)isoindolin-1-one (76)

Reaction of 5-(2-(3-(4-methylpiperazin-1-yl)propyl)-1-oxoisoindolin-5-yl)thiophene-2-carbaldehyde with 2-thiohydantoin was carried out according to general procedure D. The reaction solvent was removed under reduced pressure and the residue suspended in acetone (5 mL), to which was added sat. NaHCO₃ (5 mL). This mixture was stirred at room temperature for 1 h., the solid collected by filtration, dried, and re-suspended in MeOH (5 mL). After stirring for 10 min. at room temperature, the title compound was obtained by filtration as a dark orange solid (81%), mp (MeOH) 197-201° C. ¹H NMR [400 MHz, (CD₃)₂SO] Observe E- and Z-isomers separately. δ 11.75 (br s, 2H), 7.90 (br s, 1H), 7.82 (dd, J=8.0, 1.5 Hz, 1H), 7.77 (d, J=4.4 Hz, 0.2H), 7.65-7.72 (m, 2.8H), 6.84 (s, 0.2H), 6.51 (s, 0.8H), 4.52 (s, 2 H), 3.54 (t, J=7.0 Hz, 2H), 2.29-2.42 (m, 10H), 2.16 (s, 3H), 1.75 (pentet, J=7.1 Hz, 2 H). HRMS (ESI⁺) calcd for C₂₄H₂₅N₃O₂S₂ 482.1679 (MH⁺), found 482.1675.

### Example 184

### 5-(1-Oxo-2-(3-(pyrrolidin-1-yl)propyl)isoindolin-5-yl)thiophene-2-carbaldehyde (191)

5-(5-(Dimethoxymethyl)thiophen-2-yl)-2-(3-iodopropyl)isoindolin-1-one was reacted with pyrrolidine according to general procedure 0, followed by deprotection according to general procedure E to give the title compound as a beige solid (78%). ¹H NMR [400 MHz, (CD₃)₂SO] δ 9.94 (s, 1H), 8.08 (d, J=4.0 Hz, 1H), 8.03 (d, J=0.7 Hz, 1H), 7.92 (dd, J=8.0, 1.6 Hz, 1H), 7.86 (d, J=4.0 Hz, 1H), 7.74 (d, J=8.0 Hz, 1H), 4.55 (s, 2H), 3.57 (t, J=7.2 Hz, 2H), 2.40-2.46 (m, 6H), 1.79 (pentet, J=7.1 Hz, 2H), 1.64-1.70 (m, 4H). LRMS (APCI⁺) calcd for C₂₀H₂₃N₂O₂S 355 (MH⁺), found 355.

### Example 185

### (E,Z)-5-(5((5-Oxo-2-thioxoimidazolidin-4-ylidene)methyl)thiophen-2-yl)-2-(3-(pyrrolidin-1-yl)propyl)isoindolin-1-one (77)

Reaction of 5-(1-oxo-2-(3-(pyrrolidin-1-yl)propyl)isoindolin-5-yl)thiophene-2-carbaldehyde with 2-thiohydantoin was carried out according to general procedure D. The reaction solvent was removed under reduced pressure and the residue suspended in acetone (5 mL), to which was added sat. NaHCO₃ (5 mL). This mixture was stirred at room temperature for 1 h., the solid collected by filtration, dried, and re-suspended in MeOH (5 mL). After stirring for 10 min. at room temperature, the title compound was obtained by filtration as a dark orange solid (67%), mp (acetone) 206-210° C. ¹H NMR [400 MHz, (CD₃)₂SO] Observe E- and Z- isomers separately. □11.36 (v br s, 2H), 7.92 (br s, 0.2H), 7.90 (br s, 0.8H), 7.82 (dd, J=8.0, 1.5 Hz, 1H), 7.77 (d, J=3.9 Hz, 0.2H), 7.65-7.72 (m, 2H), 7.60 (d, J=3.9 Hz, 0.8H), 6.84 (s, 0.2H), 6.47 (s, 0.8H), 4.53 (s, 2H), 3.57 (t, J=6.9 Hz, 2H), 2.58-2.74 (m, 6H), 1.85 (pentet, J=7.2 Hz, 2H), 1.70-1.78 (m, 4H). LRMS (APCI⁺) calcd for C₂₃H₂₅N₄O₂S₂ 453 (MH⁺), found 453.

### Example 186

### 2-(2,3-Dihydroxypropyl)-5-iodoisoindolin-1-one (192) (scheme 11)

The title compound was prepared by reaction of methyl 4-iodo-2-methylbenzoate¹⁵ and 3-amino-1,2-propanediol, according to general procedure L. The desired product was isolated as a crystalline cream solid (58%). ¹H NMR [400 MHz, (CD₃)₂SO] δ 8.03 (d, J=0.7 Hz, 1H), 7.84 (dd, J=7.9, 1.4 Hz, 1H), 7.46 (d, J=7.9 Hz, 1H), 4.88 (d, J=5.2 Hz, 1H), 4.47-4.64 (m, 3H), 3.69-3.77 (m, 1H), 3.61-3.66 (m, 1H), 3.30-3.43 (m, 3H): LRMS (APCI⁺) calcd for C₁₁H₁₃INO₃ 334 (MH⁺), found 334.

### Example 187

### 5-(2-(2,3-Dihydroxypropyl)-1-oxoisoindolin-5-yl)thiophene-2-carbaldehyde (193)

2-(2,3-Dihydroxypropyl)-5-iodoisoindolin-1-one was reacted with 2-(dimethoxymethyl)thiophene according to general procedure J, then deprotected directly to the aldehyde according to general procedure E. The title compound was isolated as a yellow solid (38%). ¹H NMR [400 MHz, (CD₃)₂SO] δ 9.94 (s, 1H), 8.08 (d, J=4.0 Hz, 1H), 8.05 (d, J=0.7 Hz, 1H), 7.92 (dd, J=8.0, 1.6 Hz, 1H), 7.86 (d, J=4.0 Hz, 1H), 7.75 (d, J=8.0 Hz, 1H), 4.91 (t, J=5.2 Hz, 1H), 4.57-4.71 (m, 3H), 3.73-3.81 (m, 1H), 3.65-3.71 (m, 1H), 3.31-3.47 (m, 3H). LRMS (APCI⁺) calcd for C₁₆H₁₆NO₄S 318 (MH⁺), found 318.

### Example 188

### (E,Z)-2-(2,3-Dihydroxypropyl)-5-(5-((5-oxo-2-thioxoimidazolidin-4-ylidene)methyl)thiophen-2-yl)isoindolin-1-one (78)

Reaction of 5-(2-(2,3-dihydroxypropyl)-1-oxoisoindolin-5-yl)thiophene-2-carbaldehyde with 2-thiohydantoin was carried out according to general procedure D gave the title compound as The O,O-diacetate. Hydrolysis of the diacetate to the desired diol was carried out by treating the crude product with K₂CO₃ (5 equivalents) in a mixture of MeOH/water (5:1). The desired product was isolated in 66% yield as an orange solid, but contained an unidentified impurity (18%) which was removed by preparative HPLC; mp (DMSO/water) 260-263° C. ¹H NMR [400 MHz, (CD₃)₂SO] Observe E- and Z- isomers separately. δ 12.02-12.41 (m, 2 H), 7.94 (br s, 1H), 7.87 (br d, J=3.9 Hz, 0.7H), 7.83 (dd, J=8.0, 1.6 Hz, 1H), 7.77 (d, J=4.0 Hz, 1H), 7.72 (d, J=8.0 Hz, 1H), 7.69 (d, J=4.0 Hz, 0.3H), 6.84 (s, 0.3H), 6.65 (s, 0.7H), 4.93 (t, J=5.2 Hz, 1H), 4.56-4.59 (m, 3H), 3.72-3.80 (m, 1H), 3.63-3.70 (m, 1H), 3.33-3.46 (m, 3H). LRMS (APCI⁻) calcd for C₁₉H₁₆N₃O₄S₂ 414 (M-H), found 414.

### Example 189

### (4-Bromophenyl)(2-chloroallyl)sulfane (194)

4-Bromophenol (2.0 g, 10.58 mmol) was heated with 1M NaOH (12.0 ml) at 40° C. for 30 min. 2,3-Dichloropropene (1.16 mL, 12.69 mmol) was added and the reaction mixture was refluxed overnight. The cooled mixture was diluted with EtOAc (100 mL), washed with brine (100 mL), and dried (Na₂SO₄). Removal of the solvent afforded colourless oil (2.61 g, 94%). ¹H NMR [400 MHz, CDCl₃] δ 7.42 (d, J=8.6 Hz, 2H), 7.25 (d, J=8.6 Hz, 2H), 5.24-5.26 (m, 2H), 3.68 (d, J=0.8 Hz, 2H). LRMS (APCI⁺) calcd for C₉H₉BrClS 265 (MH⁺), found 265.

### Example 190

### 5-Bromo-2-methylbenzo[b]thiophene (195)

(4-Bromophenyl)(2-chloroallyl)sulfane (2.61 g, 9.90 mmol) was refluxed in N,N-diethylamine (20 mL) overnight. The cooled mixture was diluted with Et₂O (100 mL), washed with 1 M HCl (3×100 mL), and dried (Na₂SO₄). Removal of the solvent afforded black residue, which was purified by flash column chromatography on silica gel (hexanes as eluant) to give the title compound as a white solid (1.45 g, 64%). ¹H NMR [400 MHz, CDCl₃] δ 7.77 (d, J=1.8 Hz, 1H), 7.58 (d, J=8.5 Hz, 1H), 7.33 (dd, J=8.5, 1.9 Hz, 1H), 6.90 (s, 1H), 2.58 (d, J=1.1 Hz, 3H).

### Example 191

### 5-Bromobenzo[b]thiophene-2-carbaldehyde (196)

5-Bromo-2-methylbenzo[b]thiophene (1.45 g, 6.38 mmol) and Ce(SO₄)₂ (8.70 g, 26.18 mmol) were suspended in 50% AcOH (50 mL) and refluxed for 3 h. The cooled mixture was filtered and the filtrate extracted with Et₂O (2×50 mL). The organic extract was washed with sat. NaHCO₃ (3×100 mL) and dried (Na₂SO₄). Removal of the solvent afforded light yellow solid (0.64 g, 42%), which did not require further purification. ¹H NMR [400 MHz, CDCl₃] δ 10.11 (s, 1H), 8.09 (d, J=1.9 Hz, 1H), 7.95 (s, 1H), 7.77 (d, J=8.6 Hz, 1H), 7.60 (dd, J=8.6, 1.9 Hz, 1H).

### Example 192

### 5-Bromo-2-(dimethoxymethyl)benzo[b]thiophene (197)

5-Bromobenzo[b]thiophene-2-carbaldehyde was protected as the dimethyl acetal according to general procedure N. The title compound was isolated as a light orange solid (44%). ¹H NMR [400 MHz, CDCl₃] δ 7.89 (d, J=1.8 Hz, 1H), 7.67 (d, J=8.6 Hz, 1H), 7.41 (dd, J=8.6, 1.8 Hz, 1H), 7.25 (s, 1H), 5.68 (d, J=1.0 Hz, 1H), 3.41 (s, 6H).

### Example 193

### 5-(2-(Dimethoxymethyl)benzo[b]thiophen-5-yl)isobenzofuran-1(3H)-one (scheme 2) (198)

5-Bromo-2-(dimethoxymethyl)benzo[b]thiophene was reacted with 5-(4,4,5,5-tetramethyl-1,3,2-dioxaborolan-2-yl)isobenzofuran-1(3H)-one according to general procedure A. The title compound was obtained as a white solid (56%). ¹H NMR [400 MHz, CDCl₃] δ 8.00 (d, J=8.6 Hz, 1H), 7.98 (s, 1H), 7.93 (d, J=8.4 Hz, 1H), 7.80 (dt, J=8.0, 0.7 Hz, 1H), 7.72 (dd, J=1.2, 0.7 Hz, 1H), 7.57 (dd, J=8.4, 1.8 Hz, 1H), 7.40 (s, 1H), 5.73 (d, J=1.0 Hz, 1H), 5.39 (s, 2H), 3.44 (s, 6H). LRMS (APCI⁺) calcd for C₁₉H₁₇O₄S 341 (MH⁺), found 341.

### Example 194

### 5-(1-Oxo-1,3-dihydroisobenzofuran-5-yl)benzo[b]thiophene-2-carbaldehyde (199)

5-(2-(Dimethoxymethyl)benzo[b]thiophen-5-yl)isobenzofuran-1(31)-one was deprotected according to general procedure E to give the title compound as a white solid (99%). ¹H NMR [400 MHz, CDCl₃] δ 10.16 (s, 1H), 8.17 (d, J=1.5 Hz, 1H), 8.11 (s, 1H), 8.03 (d, J=7.9 Hz, 1H), 8.02 (d, J=8.5 Hz, 1H), 7.81 (dt, J=8.0, 0.7 Hz, 1H), 7.76 (dd, J=8.5, 1.8 Hz, 1H), 7.74 (dd, J=1.4, 0.7 Hz, 1H), 5.41 (s, 2H). LRMS (APCI⁺) calcd for C₁₇H₁₁O₃S 295 (MH⁺), found 295.

### Example 195

### (E,Z)-5-((5-(1-Oxo-1,3-dihydroisobenzofuran-5-yl)benzo[b]thiophen-2-yl)methylene)-2-thioxoimidazolidin-4-one (79)

Reaction of 5-(1-oxo-1,3-dihydroisobenzofuran-5-yl)benzo[b]thiophene-2-carbaldehyde with 2-thiohydantoin was carried out according to general procedure D to give the title compound as a bright yellow solid (90%); mp (AcOH)>305° C. ¹H NMR [400 MHz, (CD₃)₂SO] Observe E- and Z- isomers separately. δ 12.03-12.60 (br m, 2H), 8.20 (d, J=1.6 Hz, 1H), 8.19 (s, 1H), 8.14 (d, J=8.5 Hz, 1H), 8.11 (s, 0.1H), 8.04 (s, 0.9H), 7.95-7.99 (m, 2H), 7.79 (dd, J=8.5, 1.8 Hz, 1H), 6.92 (s, 0.1H), 6.66 (s, 0.9H), 5.50 (s, 2H). HRMS (ESI⁻) calcd for C₂₀H₁₁N₂O₃S₂ 391.0217 (M-H), found 391.0218.

### Example 196

### (3-Bromophenyl)(2-chloroallyl)sulfane (200)

The title compound was prepared from alkylation of 3-bromophenol with 2,3-dichloropropene in exactly the same manner as for compound 191. The desired product was isolated as a yellow oil (99%). ¹H NMR [400 MHz, CDCl₃] δ 7.52 (t, J=1.8 Hz, 1H), 7.28-7.37 (m, 2H), 7.16 (t, J=7.9 Hz, 1H), 5.31 (dd, J=2.6, 1.1 Hz, 1H), 5.27 (d, J=1.6 Hz, 1H), 3.72 (d, J=0.8 Hz, 2H).

### Example 197

### 6-Bromo-2-methylbenzo[b]thiophene (201)

The title compound was prepared from (3-bromophenyl)(2-chloroallyl)sulfane in exactly the same manner as for compound 192. The desired product was isolated as a white solid (35%). ¹H NMR [400 MHz, CDCl₃] δ 7.87 (d, J=1.7 Hz, 1H), 7.48 (d, J=8.4 Hz, 1H), 7.38 (dd, J=8.4, 1.8 Hz, 1H), 6.92 (s, 1H), 2.56 (d, J=1.2 Hz, 3H).

### Example 198

### 6-Bromobenzo[b]thiophene-2-carbaldehyde (202)

The title compound was prepared by oxidation of 6-bromo-2-methylbenzo[b]thiophene in exactly the same manner as for compound 193. The desired compound was isolated as a light orange solid (78%). ¹H NMR [400 MHz, CDCl₃] δ 10.10 (s, 1H), 8.07 (d, J=1.7 Hz, 1H), 7.98 (d, J=0.5 Hz, 1H), 7.79 (d, J=8.6 Hz, 1H), 7.55 (dd, J=8.6, 1.7 Hz, 1H).

### Example 199

### 6-Bromo-2-(dimethoxymethyl)benzo[b]thiophene (203)

6-Bromobenzo[b]thiophene-2-carbaldehyde was protected as the dimethyl acetal according to general procedure N. The title compound was isolated a yellow solid (61%). ¹H NMR [400 MHz, CDCl₃] δ 7.96 (d, J=1.8 Hz, 1H), 7.59 (d, J=8.5 Hz, 1H), 7.44 (dd, J=8.5, 1.8 Hz, 1H), 7.27 (s, 1H), 5.67 (d, J=1.0 Hz, 1H), 3.41 (s, 6H).

### Example 200

### 5-(2-(Dimethoxymethyl)benzo[b]thiophen-6-yl)isobenzofuran-1(3H)-one (204)

6-Bromo-2-(dimethoxymethyl)benzo[b]thiophene was reacted with 5-(4,4,5,5-tetramethyl-1,3,2-dioxaborolan-2-yl)isobenzofuran-1(3H)-one according to general procedure A. The title compound was obtained as a white solid (72%). ¹H NMR [400 MHz, CDCl_{3]} δ 8.08 (d, J=0.8 Hz, 1H), 8.00 (d, J=8.0 Hz, 1H), 7.85 (d, J=8.3 Hz, 1H), 7.81 (dd, J=8.0, 0.7 Hz, 1H), 7.73 (s, 1H), 7.60 (dd, J=8.3, 1.6 Hz, 1H), 7.37 (s, 1H), 5.73 (s, 1H), 5.39 (s, 2H), 3.44 (s, 6H). LRMS (APCI⁺) calcd for C₁₉H₁₇O₄S 341 (MH⁺), found 341.

### Example 201

### 6-(1-Oxo-1,3-dihydroisobenzofuran-5-yl)benzo[b]thiophene-2-carbaldehyde (205)

5-(2-(Dimethoxymethyl)benzo[b]thiophen-6-yl)isobenzofuran-1(3H)-one was deprotected according to general procedure E to give the title compound as a white solid (76%). ¹H NMR [400 MHz, CDCl_{3]} δ 10.15 (s, 1H), 8.15 (d, J=1.5 Hz, 1H), 8.08 (s, 1H), 8.06 (d, J=8.4 Hz, 1H), 8.03 (d, J=8.0 Hz, 1H), 7.83 (dd, J=8.0, 1.2 Hz, 1H), 7.76 (s, 1H), 7.70 (dd, J=8.4, 1.6 Hz, 1H), 5.40 (s, 2H). LRMS (APCI⁺) calcd for C₁₇H₁₁O₃S 295 (MH⁺), found 295.

### Example 202

### (E,Z)-5-((6-(1-Oxo-1,3-dihydroisobenzofuran-5-yl)benzo[b]thiophen-2-yl)methylene)-2-thioxoimidazolidin-4-one (80)

Reaction of 6-(1-oxo-1,3-dihydroisobenzofuran-5-yl)benzo[b]thiophene-2-carbaldehyde with 2-thiohydantoin was carried out according to general procedure D to give the title compound as a brown solid (40%); mp (AcOH)>310° C. ¹H NMR [400 MHz, (CD₃)₂SO] Observe E- and Z- isomers separately. δ 12.05-12.58 (br m, 2H), 8.44 (d, J=1.5 Hz, 1H), 8.17 (s, 1H), 8.07 (s, 1H), 7.94-8.01 (m, 3H), 7.82 (dd, J=8.4, 1.7 Hz, 1H), 6.93 (s, 0.04H), 6.64 (s, 0.95H), 5.49 (s, 2H). HRMS (ESI⁻) calcd for C₂₀H₁₁N₂O₃S₂ 391.0217 (M-H), found 391.0213.

### Example 203

### Methyl 4-bromophenethylcarbamate (206)

The title compound was prepared by adapting a literature procedure¹⁶ and was isolated as a pale yellow oil (94%). ¹H NMR [400 MHz, CDCl₃] δ 7.43 (d, J=8.4 Hz, 2H), 7.06 (d, J=8.3 Hz, 2H), 4.65 (bs, 1H), 3.65 (s, 3H), 3.41 (q, J=6.3 Hz, 2H), 2.77 (t, J=7.0 Hz, 2H). LRMS (APCI⁺) calcd for C₁₀H₁₃BrNO₂ 259 (MH⁺), found 259.

### Example 204

### 7-Bromo-3,4-dihydroisoquinolin-1(2H)-one (207)

Cyclisation of methyl 4-bromophenethylcarbamate was carried out by adaption of a literature procedure¹⁷ to give the title compound as a white solid (20%). ¹H NMR [400 MHz, CDCl_{3]} δ 8.20 (d, J=2.1 Hz, 1H), 7.55 (dd, J=8.1, 2.2 Hz, 1H), 7.09 (d, J=8.1 Hz, 1H), 6.12 (bs, 1H), 3.55 (dt, J=6.2, 2.9 Hz, 2H), 2.94 (t, J=6.6 Hz, 2H). LRMS (APCI⁺) calcd for C₉H₉BrNO 227 (MH⁺), found 227.

### Example 205

### 7-(4,4,5,5-Tetramethyl-1,3,2-dioxaborolan-2-yl)-3,4-dihydroisoquinolin-1(2H)-one (208)

The title compound was prepared by reaction of 7-bromo-3,4-dihydroisoquinolin-1(2H)-one with bis(pinacolato)diboron according to general procedure M, and was isolated as a pale brown solid (47%). ¹H NMR [400 MHz, CDCl₃] δ 8.53 (s, 1H), 7.86 (dd, J=7.5, 51.3 Hz, 1H), 7.21 (d, J=7.4 Hz, 1H), 5.88 (bs, 1H), 3.55 (dt, J=6.6, 2.9 Hz, 2H), 3.01 (t, J=6.6 Hz, 2H), 1.33 (s, 12H). LRMS (APCI⁺) calcd for C₁₅H₂₁BNO₃ 274 (MH⁺), found 274.

### Example 206

### 7-(5-(1,3-Dioxolan-2-yl)thiophen-2-yl)-3,4-dihydroisoquinolin-1(2H)-one (209)

2-(5-Bromothiophen-2-yl)-1,3-dioxolane was reacted with 7-(4,4,5,5-tetramethyl-1,3,2-dioxaborolan-2-yl)-3,4-dihydroisoquinolin-1(2H)-one according to general procedure A.

The title compound was obtained as a brown solid (41%). ¹H NMR [400 MHz, CDCl₃] δ 8.31 (d, J=2.0 Hz, 1H), 7.65 (dd, J=7.9, 2.1 Hz, 1H), 7.27 (d, J=3.6 Hz, 1H), 7.22 (d, J=8.0 Hz, 1H), 7.13 (d, J=3.8 Hz, 1H), 6.11 (s, 1H), 6.01 (bs, 1H), 4.03-4.16 (m, 4H), 3.59 (dt, J=6.7, 2.9 Hz, 2H), 3.01 (t, J=6.6 Hz, 2H). LRMS (APCI⁺) calcd for C₁₆H₁₆NO₃S 302 (MH⁺), found 302.

### Example 207

### 5-(1-Oxo-1,2,3,4-tetrahydroisoquinolin-7-yl)thiophene-2-carbaldehyde (210)

Deprotection of 7-(5-(1,3-dioxolan-2-yl)thiophen-2-yl)-3,4-dihydroisoquinolin-1(2H)-one was carried out according to general procedure B and the title compound was obtained as an orange solid (84%). ¹H NMR [400 MHz, CDCl_{3]} δ 9.90 (s, 1H), 8.40 (d, J=2.0 Hz, 1H), 7.75 (d, J=3.9 Hz, 1H), 7.73 (d, J=2.0 Hz, 1H), 7.49 (d, J=3.9 Hz, 1H), 7.30 (d, J=7.9 Hz, 1H), 6.12 (bs, 1H), 3.61 (dt, J=6.6, 2.8 Hz, 2H), 3.05 (t, J=6.6 Hz, 2H). LRMS (APCI⁺) calcd for C₁₄H₁₂NO₂S 258 (MH⁺), found 258.

### Example 208

### (E,Z)-7-(5-((5-Oxo-2-thioxoimidazolidin-4-ylidene)methyl)thiophen-2-yl)-3,4-dihydroisoquinolin-1(2H)-one (81)

Reaction of 5-(1-oxo-1,2,3,4-tetrahydroisoquinolin-7-yl)thiophene-2-carbaldehyde with 2-thiohydantoin was carried out according to general procedure D to give the title compound as a brown solid (72%); mp (AcOH)>310° C. ¹H NMR [400 MHz, (CD₃)₂SO] Observe E- and Z- isomers separately. δ 12.35-12.69 (br m, 2H), 8.12 (d, J=2.0 Hz, 0.9H), 8.10 (d, J=2.0 Hz, 0.1H), 8.03 (m, 1H), 7.86 (dd, J=7.9, 2.1 Hz, 1H), 7.79 (d, J=3.9 Hz, 0.9H), 7.74 (d, J=4.0 Hz, 0.1H), 7.66 (d, J=4.0 Hz, 0.9H), 7.59 (d, J=4.0 Hz, 0.1H), 7.40 (d, J=8.0 Hz, 1H), 6.65 (br s, 0.9H), 6.60 (br s, 0.1H), 3.40 (dt, J=6.6, 2.5 Hz, 2H), 2.93 (t, J=6.5 Hz, 2H). HRMS (ESI⁺) calcd for C₁₇H₁₄N₃O₂S₂ 356.0516 (MH⁺), found 356.0522.

### Example 209

### Methyl 3-bromophenethylcarbamate (211)

The title compound was prepared by adapting a literature procedure¹⁶ and was isolated as a pale yellow oil (96%). ¹H NMR [400 MHz, CDCl₃] δ 7.33-7.38 (m, 2H), 7.17 (t, J=7.6 Hz, 1H), 7.11 (d, J=7.7 Hz, 1H), 4.68 (bs, 1H), 3.67 (s, 3H), 3.42 (q, J=6.6 Hz, 2H), 2.78 (t, J=7.0 Hz, 2H). LRMS (APCI⁺) calcd for C₁₀H₁₃BrNO₂ 259 (MH), found 259.

### Example 210

### 6-Bromo-3,4-dihydroisoquinolin-1(2H)-one (212)

Cyclisation of methyl 3-bromophenethylcarbamate was carried out by adaption of a literature procedure¹⁷ to give the title compound as a white solid (30%). ¹H NMR [400 MHz, CDCl_{3]} δ 7.93 (d, J=8.3 Hz, 1H), 7.49 (dd, J=8.3, 1.9 Hz, 1H), 7.39 (d, J=1.7 Hz, 1H), 5.98 (bs, 1H), 3.57 (dt, J=6.6, 2.9 Hz, 2H), 2.99 (t, J=6.6 Hz, 2H). LRMS (APCI⁺) calcd for C₉H₉BrNO 227 (MH⁺), found 227.

### Example 211

### 6-(4,4,5,5-Tetramethyl-1,3,2-dioxaborolan-2-yl)-3,4-dihydroisoquinolin-1(2H)-one (213)

The title compound was prepared by reaction of 6-bromo-3,4-dihydroisoquinolin-1(2H)-one with bis(pinacolato)diboron according to general procedure M, and was isolated as a light brown solid (26%). ¹H NMR [400 MHz, CDCl₃] δ 8.06 (d, J=7.7 Hz, 1H), 7.79 (d, J=7.7 Hz, 1H), 7.66 (s, 1H), 5.95 (bs, 1H), 3.55 (dt, J=6.6, 2.9 Hz, 2H), 3.01 (t, J=6.7 Hz, 2H), 1.36 (s, 12H). LRMS (APCI⁺) calcd for C₁₅H₂₁BNO₃ 274 (MH⁺), found 274.

### Example 212

### 6-(5-(1,3-Dioxolan-2-yl)thiophen-2-yl)-3,4-dihydroisaquinolin-1(2H)-one (214)

2-(5-Bromothiophen-2-yl)-1,3-dioxolane was reacted with 6-(4,4,5,5-tetramethyl-1,3,2-dioxaborolan-2-yl)-3,4-dihydroisoquinolin-1(2H)-one according to general procedure A. The title compound was obtained as a light orange solid (44%). ¹H NMR [400 MHz, CDCl₃] δ 8.07 (d, J=8.1 Hz, 1H), 7.57 (dd, J=8.1, 1.8 Hz, 1H), 7.42 (d, J=1.8 Hz, 1H), 7.27 (d, J=3.7 Hz, 1H), 7.15 (dd, J=3.6, 0.4 Hz, 1H), 6.11 (s, 1H), 6.02 (bs, 1H), 4.03-4.21 (m, 4H), 3.59 (dt, J=6.6, 2.9 Hz, 2H), 3.03 (t, J=6.7 Hz, 2H). LRMS (APCI⁺) calcd for C₁₆H₁₆NO₃S 302 (MH⁺), found 302.

### Example 213

### 5-(1-Oxo-1,2,3,4-tetrahydroisoquinolin-6-yl)thiophene-2-carbaldehyde (215)

Deprotection of 6-(5-(1,3-dioxolan-2-yl)thiophen-2-yl)-3,4-dihydroisoquinolin-1(2H)-one was carried out according to general procedure B and the title compound was obtained as an orange solid (97%). ¹H NMR [400 MHz, CDCl₃] δ 9.92 (s, 1H), 8.13 (d, J=8.1 Hz, 1H), 7.77 (d, J=4.0 Hz, 1H), 7.66 (dd, J=8.1, 1.8 Hz, 1H), 7.52 (d, J=1.0 Hz, 1H), 7.48 (d, J=3.9 Hz, 1H), 6.01 (bs, 1H), 3.62 (dt, J=6.6, 2.9 Hz, 2H), 3.07 (t, J=6.6 Hz, 2H). LRMS (APCI⁺) calcd for C₁₄H₁₂NO₂S 258 (MH⁺), found 258.

### Example 214

### (E,Z)-6-(5-((5-Oxo-2-thioxoimidazolidin-4-ylidene)methyl)thiophen-2-yl)-3,4-dihydroisoquinolin-1(2H)-one (82)

Reaction of 5-(1-oxo-1,2,3,4-tetrahydroisoquinolin-6-yl)thiophene-2-carbaldehyde with 2-thiohydantoin was carried out according to general procedure D to give the title compound as a brown solid (65%); mp (AcOH) 341-344° C. ¹H NMR [400 MHz, (CD₃)₂SO] Observe E- and Z- isomers separately. δ 11.98-12.30 (br m, 2H), 7.93 (br s, 1H), 7.88 (d, J=8.1 Hz, 1H), 7.83 (d, J=3.9 Hz, 1H), 7.77 (d, J=4.4 Hz, 0.2H), 7.74 (d, J=4.0 Hz, 0.8H), 7.64-7.70 (m, 2H), 6.83 (br s, 0.2H), 6.63 (br s, 0.8H), 3.40 (dt, J=6.6, 2.5 Hz, 2H), 2.96 (t, J=6.5 Hz, 2H). HRMS (ESI⁻) calcd for C₁₇H₁₂N₃O₂S₂ 354.0376 (M-H), found 354.0368.

### Example 215

### 2-(4-Bromophenyl)-N-methylethanamine (216)

The title compound was prepared by adapting a literature procedure and was isolated as a yellow solid (46%). ¹H NMR [400 MHz, CDCl₃] δ 7.41 (d, J=8.4 Hz, 2H), 7.08 (d, J=8.3 Hz, 2H), 2.73-2.86 (m, 4H), 2.43 (s, 3H), 1.48 (bs, 1H). LRMS (APCI⁺) calcd for C₉H₁₂BrN 215 (MH⁺), found 215.

### Example 216

### Methyl 4-bromophenethyl(methyl)carbamate (217)

The title compound was prepared by adapting a literature procedure¹⁶ and was isolated as an orange oil (89%). ¹H NMR [400 MHz, CDCl₃] δ 7.42 (d, J=8.4 Hz, 2H), 7.04-7.09 (m, 2H), 3.65 (s, 3H), 3.45 (s, 3H), 2.72-2.81 (m, 4H). LRMS (APCI⁺) calcd for C₁₁H₁₄BrNO₂ 273 (MH⁺), found 273.

### Example 217

### 7-Bromo-2-methyl-3,4-dihydroisoquinolin-1(2H)-one (218)

Cyclisation of methyl 4-bromophenethyl(methyl)carbamate was carried out by adaption of a literature procedure¹⁷ to give the title compound as an orange oil (55%). ¹H NMR [400 MHz, CDCl₃] δ 8.21 (d, J=2.1 Hz, 1H), 7.52 (dd, J=8.1, 2.2 Hz, 1H), 7.05 (d, J=8.0 Hz, 1H), 3.56 (t, J=6.7 Hz, 2H), 3.15 (s, 3H), 2.95 (t, J=6.7 Hz, 2H). LRMS (APCI⁺) calcd for C₁₀H₁₀BrNO 241 (MH⁺), found 241.

### Example 218

### 2-Methyl-7-(4,4,5,5-tetramethyl-1,3,2-dioxaborolan-2-yl)-3,4-dihydroisoquinolin-1(2H)-one (219)

The title compound was prepared by reaction of 7-bromo-2-methyl-3,4-dihydroisoquinolin-1(2H)-one with bis(pinacolato)diboron according to general procedure M, and was isolated as a pale yellow oil (58%). ¹H NMR [400 MHz, CDCl₃] A 8.53 (s, 1H), 7.82 (dd, J=7.5, 1.3 Hz, 1H), 7.16 (d, J=7.5 Hz, 1H), 3.55 (t, J=6.7 Hz, 2H), 3.15 (s, 3H), 3.01 (t, J=6.6 Hz, 2H), 1.33 (s, 12H). LRMS (APCI⁺) calcd for C₁₆H₂₂BNO₃ 288 (MH), found 288.

### Example 219

### 7-(5-(1,3-Dioxolan-2-yl)thiophen-2-yl)-2-methyl-3,4-dihydroisoquinolin-1(2H)-one (220)

2-(5-Bromothiophen-2-yl)-1,3-dioxolane was reacted with 2-methyl-7-(4,4,5,5-tetramethyl-1,3,2-dioxaborolan-2-yl)-3,4-dihydroisoquinolin-1(2H)-one according to general procedure A. The title compound was obtained as a yellow oil (41%). ¹H NMR [400 MHz, CDCl₃] δ 8.32 (d, J=2.0 Hz, 1H), 7.61 (dd, J=7.8, 2.0 Hz, 1H), 7.25 (d, J=3.6 Hz, 1H), 7.17 (d, J=7.8 Hz, 1H), 7.12 (d, J=3.6 Hz, 1H), 6.11 (s, 1H), 4.01-4.17 (m, 4H), 3.58 (t, J=6.7 Hz, 2H), 3.17 (s, 3H), 3.01 (t, J=6.7 Hz, 2H). LRMS (APCI⁺) calcd for C₁₇H₁₇NO₃S 316 (MH), found 316.

### Example 220

### 5-(2-Methyl-1-oxo-1,2,3,4-tetrahydroisoquinolin-7-yl)thiophene-2-carbaldehyde (221)

Deprotection of 7-(5-(1,3-dioxolan-2-yl)thiophen-2-yl)-2-methyl-3,4-dihydroisoquinolin-1(2H)-one was carried out according to general procedure B and the title compound was obtained as a yellow-orange solid (99%). ¹H NMR [400 MHz, CDCl₃]δ 9.89 (s, 1H), 8.41 (d, J=2.0 Hz, 1H), 7.74 (d, J=4.0 Hz, 1H), 7.69 (dd, J=7.8, 2.1 Hz, 1H), 7.48 (d, J=4.0 Hz, 1H), 7.24 (s, 1H), 3.60 (t, J=6.7 Hz, 2H), 3.19 (s, 3H), 3.05 (t, J=6.7 Hz, 2H). LRMS (APCI⁺) calcd for C₁₅H₁₃NO₂S 272 (MH⁺), found 272.

### Example 221

### (E,Z)-2-Methyl-7-(5-((5-oxo-2-thioxoimidazolidin-4-ylidene)methyl)thiophen-2-yl)-3,4-dihydroisoquinolin-1(2H)-one (83)

Reaction of 5-(2-methyl-1-oxo-1,2,3,4-tetrahydroisoquinolin-7-yl)thiophene-2-carbaldehyde with 2-thiohydantoin was carried out according to general procedure D to give the title compound as a red-brown solid (66%); mp (AcOH)>320° C. ¹H NMR [400 MHz, (CD₃)₂SO] Observe E- and Z- isomers separately. δ 11.72-12.49 (br m, 2H), 8.13 (d, J=2.0 Hz, 0.7H), 8.12 (d, J=2.0 Hz, 0.3H), 7.84 (dd, J=7.9, 2.2 Hz, 0.7H), 7.83 (dd, J=7.9, 2.2 Hz, 0.3H), 7.79 (d, J=4.0 Hz, 0.7H), 7.73 (d, J=4.0 Hz, 0.3H), 7.65 (d, J=3.9 Hz, 0.7H), 7.58 (d, J=4.0 Hz, 0.3H), 7.38 (d, J=7.8 Hz, 1H), 6.84 (s, 0.3H), 6.64 (s, 0.7H), 3.58 (t, J=6.7 Hz, 2H), 3.06 (s, 3H), 3.01 (t, J=6.6 Hz, 2H). HRMS (ESI⁻) calcd for C₁₈H₁₄N₃O₂S₂ 368.0533 (M-H), found 368.0529.

### Example 222

### 2-(3-Bromophenyl)-N-methylethanamine (222)

The title compound was prepared by adapting a literature procedure¹⁸ and was isolated as a pale yellow oil (68%). ¹H NMR [400 MHz, CDCl₃] δ 7.26-7.36 (m, 2H), 7.22 (s, 1H), 7.16-7.20 (m, 1H), 2.76-2.87 (m, 4H), 2.44 (s, 3H), 1.52 (bs, 1H). LRMS (APCI⁺) calcd for C₉H₁₂BrN 215 (MH⁺), found 215.

### Example 223

### Methyl 3-bromophenethyl(methyl)carbamate (223)

The title compound was prepared by adapting a literature procedure and was isolated as a yellow oil (88%). ¹H NMR [400 MHz, CDCl₃] δ 7.32-7.38 (m, 1H), 7.30 (d, J=7.4 Hz, 1H), 7.16-7.24 (m, 2H), 3.69 (s, 3H), 3.48 (s, 3H), 2.72-2.91 (m, 4H). LRMS (APCI⁺) calcd for C₁₁H₁₄BrNO₂ 273 (MH⁺), found 273.

### Example 224

### 6-Bromo-2-methyl-3,4-dihydroisoquinolin-1(2H)-one (224)

Cyclisation of methyl 3-bromophenethyl(methyl)carbamate was carried out by adaption of a literature procedure¹⁷ to give the title compound as a pale orange oil (13%). ¹H NMR [400 MHz, CDCl₃] δ 7.94 (d, J=8.3 Hz, 1H), 7.46 (dd, J=8.3, 1.8 Hz, 1H), 7.34 (d, J=1.8 Hz, 1H), 3.56 (t, J=6.8 Hz, 2H), 3.14 (s, 3H), 2.98 (t, J=6.9 Hz, 2H). LRMS (APCI⁺) calcd for C₁₀H₁₀BrNO 241 (MH⁺), found 241.

### Example 225

### 2-Methyl-6-(4,4,5,5-tetramethyl-1,3,2-dioxaborolan-2-yl)-3,4-dihydroisoquinolin-1(2H)-one (225)

The title compound was prepared by reaction of 6-bromo-2-methyl-3,4-dihydroisoquinolin-1(2H)-one with bis(pinacolato)diboron according to general procedure M, and was isolated as a yellow oil (0.26 g, 86%). ¹H NMR [400 MHz, CDCl₃] δ 8.09 (dd, J=7.5, 1.0 Hz, 1H), 7.76 (d, J=7.6 Hz, 1H), 7.61 (s, 1H), 3.57 (t, J=6.8 Hz, 2H), 3.16 (s, 3H), 3.01 (t, J=6.6 Hz, 2H), 1.35 (s, 12H). LRMS (APCI⁺) calcd for C₁₆H₂₂BNO₃ 288 (MH⁺), found 288.

### Example 226

### 6-(5-(1,3-Dioxolan-2-yl)thiophen-2-yl)-2-methyl-3,4-dihydroisoquinolin-1(2H)-one (226)

2-(5-Bromothiophen-2-yl)-1,3-dioxolane was reacted with 2-methyl-6-(4,4,5,5-tetramethyl-1,3,2-dioxaborolan-2-yl)-3,4-dihydroisoquinolin-1(2H)-one according to general procedure A. The title compound was obtained as an orange-brown solid (88%). ¹H NMR [400 MHz, CDCl₃] δ 8.08 (d, J=8.1 Hz, 1H), 7.55 (dd, J=7.8, 1.4 Hz, 1H), 7.37 (s, 1H), 7.25 (d, J=3.6 Hz, 1H), 7.14 (d, J=3.6 Hz, 1H), 6.11 (s, 1H), 4.01-4.21 (m, 4H), 3.61 (t, J=6.7 Hz, 2H), 3.18 (s, 3H), 3.03 (t, J=6.7 Hz, 2H). LRMS (APCI⁺) calcd for C₁₇H₁₇NO₃S 316 (MH⁺), found 316.

### Example 227

### 5-(2-Methyl-1-oxo-1,2,3,4-tetrahydroisoquinolin-6-yl)thiophene-2-carbaldehyde (227)

Deprotection of 6-(5-(1,3-dioxolan-2-yl)thiophen-2-yl)-2-methyl-3,4-dihydroisoquinolin-1(2H)-one was carried out according to general procedure B and the title compound was obtained as an orange solid (75%). ¹H NMR [400 MHz, CDCl₃] δ 9.91 (s, 1H), 8.14 (d, J=8.1 Hz, 1H), 7.75 (d, J=4.0 Hz, 1H), 7.64 (dd, J=8.1, 1.9 Hz, 1H), 7.47 (s, 1H), 7.46 (d, J=4.0 Hz, 1H), 3.61 (t, J=6.6 Hz, 2H), 3.18 (s, 3H), 3.07 (t, J=6.6 Hz, 2H). LRMS (APCI⁺) calcd for C₁₅H₁₃NO₂S 272 (MH⁺), found 272.

### Example 228

### (E,Z)-2-Methyl-6-(5-((5-oxo-2-thioxoimidazolidin-4-ylidene)methyl)thiophen-2-yl)-3,4-dihydroisoquinolin-1(2H)-one (84)

Reaction of 5-(2-methyl-1-oxo-1,2,3,4-tetrahydroisoquinolin-7-yl)thiophene-2-carbaldehyde with 2-thiohydantoin was carried out according to general procedure D to give the title compound as an orange-brown solid (60%); mp (AcOH)>300° C. ¹H NMR [400 MHz, (CD₃)₂SO] Observe E- and Z- isomers separately. δ 12.40 (s, 0.4H), 12.28 (s, 0.6H), 12.10 (s, 0.6H), 11.98 (s, 0.4H), 7.90 (d, J=8.1 Hz, 1H), 7.86 (d, J=4.1 Hz, 0.4H), 7.76 (d, J=4.4 Hz, 0.6H), 7.74 (d, J=4.0 Hz, 0.4H), 7.69 (dd, J=8.0, 2.0 Hz, 0.4H), 7.67 (dd, J=8.1, 1.9 Hz, 0.6H), 7.65 (d, J=4.1 Hz, 0.6H), 7.64 (s, 0.4H), 7.62 (s, 0.6H), 6.83 (s, 0.6H), 6.65 (s, 0.4H), 3.57 (t, J=6.6 Hz, 2H), 3.05 (t, J=6.6 Hz, 2H), 3.04 (s, 3H). HRMS (ESI⁻) calcd for C₁₈H₁₄N₃O₂S₂ 368.0533 (M-H), found 368.0542.

TABLE 1
Elemental Analyses for selected compounds

| | | Calcd | | | Found | | |
|---|---|---|---|---|---|---|---|
| Cmpd | | C | H | N | C | H | N |
| 1 | C₁₆H₁₀NO₄S.0.75H₂O | 56.6 | 3.4 | 8.2 | 56.7 | 3.5 | 8.6 |
| 2 | C₁₆H₁₀N₂O₃S₂ | 56.1 | 2.9 | 8.2 | 56.6 | 3.1 | 7.9 |
| 3 | C₁₆H₁₀N₂O₄S | 58.9 | 3.1 | 8.6 | 59.2 | 3.2 | 8.3 |
| 4 | C₁₇H₁₃N₃O₃S.0.75H₂O | 57.9 | 4.2 | 11.0 | 57.8 | 4.2 | 10.9 |
| 5 | C₁₈H₁₂N₂O₃S.0.5H₂O | 62.6 | 3.8 | 8.1 | 63.0 | 3.9 | 8.1 |
| 6 | C₁₈H₁₂N₂O₃S | 64.3 | 3.6 | 8.3 | 64.5 | 3.7 | 8.2 |
| 7 | C₁₇H₁₁N₃O₃S | 60.5 | 3.3 | 12.5 | 60.2 | 3.4 | 12.4 |
| 8 | C₁₇H₁₁N₃O₃S | 60.5 | 3.3 | 12.5 | 60.5 | 3.4 | 12.0 |
| 9 | C₂₀H₁₂N₂O₃S₂.0.5H₂O | 59.8 | 3.3 | 7.0 | 60.0 | 3.2 | 7.0 |
| 10 | C₂₀H₁₂N₂O₄S.0.1AcOH | 63.5 | 3.3 | 7.3 | 63.2 | 3.3 | 7.3 |
| 11 | C₂₀H₁₃N₃O₃S | 64.0 | 3.5 | 11.2 | 63.8 | 3.4 | 11.0 |
| 12 | C₂₀H₁₃N₃O₃S.0.25H₂O | 63.2 | 3.6 | 11.1 | 62.9 | 3.5 | 11.0 |
| 13 | C₂₁H₁₃N₃O₃S.0.3AcOH | 64.0 | 3.5 | 10.4 | 63.8 | 3.3 | 10.5 |
| 14 | C₂₁H₁₃N₃O₃S | 64.7 | 3.2 | 10.8 | 65.1 | 3.4 | 10.9 |
| 15 | C₁₆H₁₀N₂O₃S₂.0.25H₂O | 55.4 | 3.1 | 8.1 | 55.3 | 3.0 | 7.8 |
| 16 | C₁₅H₁₁N₃O₂S₂ | 54.7 | 3.4 | 12.8 | 55.0 | 3.5 | 12.6 |
| 17 | C₁₅H₁₁N₃O₂S₂.0.5H₂O | 53.2 | 3.6 | 12.4 | 53.1 | 3.5 | 11.9 |
| 18 | C₁₇H₁₄N₂O₃S₂ | 57.0 | 3.9 | 7.8 | 57.0 | 4.0 | 7.8 |
| 19 | C₁₆H₁₂N₂O₃S₂ | 55.8 | 3.5 | 8.1 | 55.7 | 3.4 | 8.1 |
| 20 | C₁₆H₁₂N₂O₂S₂.0.25H₂O | 57.9 | 3.7 | 8.7 | 57.7 | 3.8 | 8.4 |
| 21 | C₁₇H₁₄N₂O₃S₂ | 57.0 | 3.9 | 7.8 | 56.9 | 4.0 | 7.9 |
| 23 | C₁₄H₉ClN₂OS₂.0.25H₂O | 51.7 | 2.9 | 8.6 | 51.4 | 2.8 | 8.7 |
| 24 | C₁₄H₉ClN₂OS₂ | 52.4 | 2.8 | 8.7 | 52.3 | 2.9 | 8.6 |
| 25 | C₁₃H₉N₃OS₂.1.25H₂O | 49.6 | 3.8 | 13.4 | 49.7 | 3.4 | 13.2 |
| 26 | C₁₃H₉N₃OS₂.0.25H2O | 53.5 | 3.3 | 14.4 | 53.5 | 3.4 | 14.2 |
| 27 | C₁₅H₁₂N₂OS₃ | 54.2 | 3.6 | 8.4 | 54.3 | 3.8 | 8.6 |
| 29 | C₁₄H₉FN₂OS₂ | 55.3 | 3.0 | 9.2 | 55.0 | 3.0 | 9.2 |
| 30 | C₁₄H₈F₂N₂OS₂.0.25H₂O | 51.4 | 2.6 | 8.6 | 51.5 | 2.6 | 8.5 |
| 31 | C₁₄H₉FN₂OS₂.1.25H₂O | 51.4 | 3.2 | 8.6 | 51.5 | 2.9 | 8.6 |
| 32 | C₁₄H₉BrN₂OS₂.0.5H₂O | 44.9 | 2.7 | 7.5 | 44.5 | 2.5 | 7.4 |
| 33 | C₁₅H₁₃N₃O₃S₃ | 47.5 | 3.5 | 11.1 | 47.2 | 3.5 | 11.2 |
| 34 | C₁₅H₉N₃OS₂.0.75H₂O | 55.5 | 3.3 | 12.9 | 55.6 | 3.3 | 12.4 |
| 35 | C₁₄H₁₀N₂O₂S₂ | 55.6 | 3.3 | 9.3 | 55.9 | 3.5 | 9.3 |
| 36 | C₁₇H₁₁NO₄S | 62.7 | 3.4 | 4.3 | 62.3 | 3.6 | 4.2 |
| 37 | C₁₇H₁₃NO₃S | 64.6 | 4.3 | 4.4 | 64.3 | 4.2 | 4.4 |
| 38 | C₁₆H₁₃N₃O₂S₂ | 56.0 | 3.8 | 12.2 | 55.9 | 3.9 | 12.2 |
| 39 | C₁₇H₁₅N₃O₂S₂ | 57.1 | 4.2 | 11.8 | 57.2 | 4.2 | 11.6 |
| 40 | C₁₈H₁₇N₃O₄S₂ | 53.6 | 4.3 | 10.4 | 53.3 | 4.4 | 10.3 |
| 41 | C₁₈H₁₇N₃O₃S₂ | 55.8 | 4.4 | 10.8 | 55.5 | 4.6 | 10.9 |
| 42 | C₁₇H₁₅N₃O₃S₂ | 54.7 | 4.1 | 11.3 | 54.4 | 4.1 | 11.2 |
| 44 | C₁₉H₁₇N₃O₃S₂ | 55.8 | 4.4 | 10.8 | 56.1 | 4.6 | 10.6 |
| 45 | C₂₁H₂₂N₄O₃S₂.1.5H₂O | 53.7 | 5.4 | 11.9 | 53.6 | 5.1 | 11.9 |
| 46 | C₁₆H₁₁N₃O₂S₂.1.5H₂O | 52.2 | 3.8 | 11.4 | 52.2 | 3.7 | 10.9 |
| 47 | C₁₆H₁₁N₃O₃S.1.25H₂O | 55.2 | 3.9 | 12.0 | 55.52 | 3.60 | 11.6 |
| 48 | C₁₈H₁₃N₃O₂S.0.5AcOH | 62.5 | 4.1 | 11.5 | 62.7 | 4.2 | 11.5 |
| 49 | C₁₈H₁₃N₃O₂S.0.5AcOH | 62.5 | 4.1 | 11.5 | 62.8 | 4.3 | 11.5 |
| 51 | C₁₇H₁₂N₄O₂S.0.5H₂O | 59.1 | 3.8 | 16.2 | 59.4 | 3.6 | 16.0 |
| 53 | C₁₆H₁₁N₃O₃S.0.25AcOH | 58.2 | 3.4 | 12.4 | 58.5 | 3.5 | 12.4 |
| 54 | C₁₅H₉N₃O₄S | 55.0 | 2.8 | 12.8 | 55.0 | 2.9 | 12.6 |
| 55 | C₁₅H₉N₃O₃S₂.0.25H₂O | 51.8 | 2.8 | 12.1 | 51.8 | 2.9 | 11.7 |
| 56 | C₁₅H₉N₃O₃S₂ | 52.5 | 2.6 | 12.2 | 52.6 | 2.8 | 12.2 |
| 57 | C₁₅H₉N₃O₃S₂ | 52.5 | 2.6 | 12.2 | 52.2 | 2.8 | 12.5 |
| 58 | C₁₅H₉N₃O₄S.0.5H₂O | 53.6 | 3.0 | 12.5 | 53.6 | 2.9 | 12.1 |
| 59 | C₁₅H₉N₃O₃S₂ | 52.5 | 2.6 | 12.2 | 52.5 | 2.7 | 12.0 |
| 60 | C₁₅H₉N₃O₃S₂ | 52.5 | 2.6 | 12.2 | 52.5 | 2.8 | 12.1 |
| 61 | C₁₇H₁₃N₃O₂S₂ | 57.5 | 3.7 | 11.8 | 57.6 | 3.7 | 11.6 |
| 62 | C₁₈H₁₅N₃O₂S₂.0.25H₂O | 57.8 | 4.2 | 11.2 | 57.5 | 4.1 | 11.4 |
| 64 | C₂₀H₁₇N₃O₄S₂ | 60.4 | 4.8 | 10.6 | 60.2 | 4.9 | 10.5 |
| 65 | C₁₉H₁₅N₃O₄S₂.0.25H₂O | 54.3 | 3.7 | 10.1 | 54.5 | 3.8 | 9.7 |
| 66 | C₁₈H₁₃N₃O₃S₂.0.25AcOH | 55.8 | 4.2 | 10.6 | 56.2 | 3.9 | 10.3 |
| 68 | C₂₀H₁₇N₃O₄S₂.0.5H₂0 | 55.0 | 4.2 | 9.6 | 55.1 | 4.0 | 9.6 |
| 70 | C₁₈H₁₅N₃O₃S₂.0.75H₂O | 54.2 | 4.2 | 10.5 | 54.2 | 4.3 | 10.2 |
| 71 | C₁₉H₁₇N₃O₃S₂.0.5H₂O | 55.9 | 4.4 | 10.3 | 55.8 | 4.2 | 10.2 |
| 72 | C₁₆H₁₁N₃O₂S₂ | 56.3 | 3.3 | 12.3 | 56.2 | 3.3 | 12.2 |
| 73 | C₁₇H₁₃N₃O₂S₂ | 57.5 | 3.7 | 11.8 | 57.5 | 3.7 | 11.9 |
| 74 | C₂₁H₂₂N₄O₂S₂.H₂O | 56.8 | 5.4 | 12.6 | 56.9 | 5.5 | 12.8 |
| 77 | C₂₃H₂₄N₄O₂S₂.0.5H₂O | 59.8 | 5.5 | 12.1 | 59.5 | 5.3 | 11.8 |
| 78 | C₁₉H₁₇N₃O₄S₂.0.75H₂O | 53.2 | 4.4 | 9.8 | 53.2 | 4.3 | 9.6 |
| 79 | C₂₀H₁₂N₂O₃S₂ | 61.2 | 3.1 | 7.1 | 61.1 | 3.1 | 7.0 |
| 80 | C₂₀H₁₂N₂O₃S₂.0.2AcOH | 60.6 | 3.2 | 6.9 | 60.3 | 3.0 | 7.0 |
| 81 | C₁₇H₁₃N₃O₂S₂.0.5H₂O | 56.0 | 3.9 | 11.5 | 55.8 | 3.9 | 11.6 |
| 82 | C₁₇H₁₃N₃O₂S₂.0.4H₂O | 56.3 | 3.8 | 11.6 | 56.0 | 3.8 | 11.8 |
| 83 | C₁₈H₁₅N₃O₂S₂.H₂O | 55.8 | 4.4 | 10.8 | 55.8 | 4.4 | 10.4 |
| 84 | C₁₈H₁₅N₃O₂S₂.0.1H₂O | 58.2 | 4.1 | 11.3 | 57.9 | 4.0 | 11.3 |

TABLE 2
Elemental Analyses for Selected Intermediates

| | | Calcd | | | Found | | |
|---|---|---|---|---|---|---|---|
| Cmpd | | C | H | N | C | H | N |
| 83 | C₁₃H₈O₄ | 68.4 | 3.5 | | 67.9 | 3.7 | |
| 85 | C₁₃H₈O₃S | 63.9 | 3.3 | | 63.4 | 3.4 | |
| 135 | C₁₃H₉NO₃ | 68.7 | 4.0 | 6.2 | 68.5 | 4.2 | 6.1 |
| 136 | C₁₂H₉NO₃ | 67.0 | 4.2 | 6.5 | 66.7 | 4.2 | 6.5 |
| 137 | C₁₀H₅NO₄S | 51.1 | 2.1 | 6.0 | 50.8 | 2.2 | 5.8 |

TABLE 3
HRMS and HPLC for Selected Compounds

| | HRMS | | | |
|---|---|---|---|---|
| Cmpd | Formula | Calcd. | Found | HPLC |
| 22 | C₁₅H₁₂N₂O₂S₂ (M⁺) | 316.0340 | 316.0338 | 96.7% |
| 28 | C₁₅H₁₁N₂O₃S₃ (M − H) | 362.9937 | 362.9922 | 96.9% |
| 32 | C₁₄H₈⁷⁹BrN₂OS₂ (M − H) | 362.9267 | 362.9256 | 99.6% |
| | C₁₄H₈⁸¹BrN₂OS₂ (M − H) | 364.9246 | 364.9230 | |
| 43 | C₂₂H₂₅N₄O₃S₂ (MH⁺) | 457.1363 | 457.1369 | 96.7% |
| 50 | C₁₆H₁₂N₃O₂S₂ (MH⁺) | 342.0371 | 342.0372 | 99.8% |
| 52 | C₁₉H₁₈N₃O₂S₂ (MH⁺) | 384.0835 | 384.0821 | 98.3% |
| 63 | C₁₉H₁₆N₃O₂S₂ (M − H) | 382.0689 | 382.0697 | 96.6% |
| 67 | C₂₂H₂₃N₄O₃S₂ (MH⁺) | 455.1206 | 455.1201 | 95.0% |
| 69 | C₁₈H₁₄N₃O₂S₂ (M − H) | 368.0533 | 368.0526 | 95.0% |
| 75 | C₂₄H₂₇N₄O₂S₂ (MH⁺) | 467.1570 | 467.1567 | 98.1% |
| 76 | C₂₄H₂₈N₅O₂S₂ (MH⁺) | 482.1679 | 482.1675 | 95.1% |

### Example 229

### Biological Activity of Exemplary Compounds of the Invention

Inhibition of Perforin-Mediated Lysis of Jurkat Cells

The ability of the compounds to inhibit the lysis of nucleated (Jurkat T lymphoma) cells in the presence of 0.1% BSA, as measured by release of ⁵¹Cr was measured. Jurkat target cells were labelled by incubation in medium with 100 μCi ⁵¹Cr for one hour. The cells were then washed three times to remove unincorporated isotope and re-suspended at 1×10⁵ cells per mL in RPMI buffer supplemented with 0.1% BSA. Each test compound was pre-incubated to concentrations of 20 μM, 10 μM, 5 μM, 2.5 μM and 1.25 μM with recombinant perforin for 30 minutes with DMSO as a negative control. ⁵¹Cr labeled Jurkat cells were then added and cells were incubated at 37° C. for 4 hours. The supernatant was collected and assessed for its radioactive content on a gamma counter (Wallac Wizard 1470 automatic gamma counter). Each data point was performed in triplicate and an IC₅₀ was calculated from the range of concentrations described to above. Compounds with an IC₅₀<1 μM were titrated down to lower concentrations in the same manner as above, to determine an accurate IC₅₀.

Inhibition of KHYG-1 NK Cell-Mediated lysis of K562 Cells

KHYG-1 cells were washed and resuspended in RPMI+0.1% BSA at 4×10⁵ cells/ml and 50 μl of KHYG-1 cells were dispensed to each well of a 96-well V-bottom, plate. Test compounds were added to KHYG-1 cells at various concentrations up to 20 μM and incubated at RT for 20 minutes. 1×10⁶ K562 target cells were labelled with 75 μCi ⁵¹Cr in 200 μl RPMI for 90 mins at 37° C., cells were washed as described above and resuspended in 5 ml RPMI+0.1% BSA. 50 μl of ⁵¹Cr labelled K562 leukemia target cells were added to each well of the KHYG-1 plate (Effector:Target 2:1) and incubated at 37° C. for 4 hours. ⁵¹Cr release was assayed using a Skatron Harvesting Press and radioactivity estimated on a Wallac Wizard 1470 Automatic Gamma counter (Turku, Finland). The percentage of specific cytotoxicity was calculated by the formula:

$$\% specific lysis = \frac{( experimental release - spontaneous release )}{( maximum release - spontaneous release )} \times 100$$

and expressed as the mean of triplicate assays +/−standard error of the mean.

Toxicity to KHYG-1 NK Cells

KHYG-1 cells were washed and resuspended in RPMI+0.1% BSA at 4×10⁵ cells/ml and 50 μl of KHYG-1 cells were dispensed to each well of a 96-well V-bottom plate. Test compounds were added to KHYG-1 cells at a concentration of 20 μM and incubated at RT for 20 minutes. 100 μL of RPMI 0.1% BSA was added and the cells were incubated for 4 hours at 37° C. then washed ×3 in RPMI+0.1% BSA. Cells were then resuspended in 200 μl of complete medium and incubated for 18 to 24 hours at 37° C. Trypan blue was added to each well and viable (clear) cells counted as a percentage of total (clear+blue) cell number (% viability).

TABLE 4
Biological Activity of Selected Compounds.

| | | | Toxicity to |
|---|---|---|---|
| | Jurkat | Inhibition of | KHYG-1 left |
| | IC₅₀ | KHYG-1 at | for 24 h at 20 |
| Cmpd | (μM) | 20 μM (%) | μM (%) |
| 1 | 6.20 | 95 | 66 |
| 2 | 0.76 | 52 | 83 |
| 3 | 1.19 | 70 | 82 |
| 4 | 2.75 | 50 | 100 |
| 5 | 2.11 | 64 | 97 |
| 6 | 1.80 | 50 | 95 |
| 7 | 2.00 | | |
| 8 | 0.37 | 57 | 96 |
| 9 | 2.27 | 80 | 0 |
| 10 | 0.62 | 51 | 88 |
| 11 | 2.34 | 100 | 86 |
| 12 | 0.72 | 74 | 94 |
| 13 | 1.72 | 52 | 70 |
| 14 | 0.47 | 56 | 81 |
| 15 | 1.05 | 67 | 96 |
| 16 | 1.56 | 80 | 52 |
| 17 | 0.79 | 35 | 100 |
| 18 | 3.04 | 98 | 100 |
| 19 | 1.62 | 30 | 95 |
| 20 | 1.67 | 90 | 100 |
| 21 | 3.03 | 92 | |
| 22 | 8.93 | 78 | 100 |
| 23 | 7.19 | 92 | 55 |
| 24 | 5.22 | 59 | 89 |
| 25 | 10.06 | | |
| 26 | 12.89 | | |
| 27 | 1.15 | 43 | 83 |
| 28 | 13.80 | | |
| 29 | 6.69 | | |
| 30 | 4.59 | | |
| 31 | 10.77 | | |
| 32 | 1.69 | 66 | 83 |
| 33 | 7.70 | 32 | |
| 34 | 9.68 | 97 | 99 |
| 35 | 7.72 | 90 | 93 |
| 36 | 6.97 | 51 | 65 |
| 37 | 6.81 | 20 | 97 |
| 38 | 4.53 | 49 | 97 |
| 39 | 6.93 | 89 | 95 |
| 40 | 2.44 | 47 | 82 |
| 41 | 6.50 | 60 | 91 |
| 42 | 3.64 | 42 | |
| 43 | 8.13 | 81 | 89 |
| 44 | 4.35 | 52 | |
| 45 | >20 | 78 | 88 |
| 46 | 2.55 | 70 | 98 |
| 47 | 0.40 | 73 | 92 |
| 48 | 5.55 | 16 | |
| 49 | 3.44 | 8 | |
| 50 | 3.00 | 42 | |
| 51 | 0.63 | 19 | 97 |
| 52 | 4.42 | 77 | 92 |
| 53 | 2.45 | 30 | |
| 54 | 7.28 | 55 | 100 |
| 55 | 1.72 | 53 | 95 |
| 56 | 1.41 | | |
| 57 | 0.34 | | |
| 58 | 1.30 | 39 | 82 |
| 59 | 2.54 | 24 | 93 |
| 60 | 1.44 | 45 | |
| 61 | 0.51 | 83 | 76 |
| 62 | 0.60 | 84 | 79 |
| 63 | 1.52 | 95 | 94 |
| 64 | 0.99 | 96 | 94 |
| 65 | 0.99 | 72 | 96 |
| 66 | 0.93 | 49 | 85 |
| 67 | | 78 | 73 |
| 68 | 1.17 | 86 | 84 |
| 69 | 3.21 | 100 | 95 |
| 70 | 0.78 | 38 | 77 |
| 71 | 0.53 | 62 | 88 |
| 72 | 1.38 | 58 | 81 |
| 73 | 1.89 | 60 | 85 |
| 74 | >20 | 53 | 82 |
| 75 | 11.78 | 61 | 85 |
| 76 | >20 | 58 | |
| 77 | >20 | 81 | 80 |
| 78 | 1.26 | 19 | |
| 79 | 0.49 | 59 | 72 |
| 80 | 0.36 | 55 | 75 |
| 81 | 0.67 | 62 | 88 |
| 82 | 1.14 | 92 | 80 |
| 83 | 4.76 | 91 | |
| 84 | 2.33 | 72 | |

### REFERENCES

1. Lu, T.-J.; Yang, J.-F.; Sheu, L.-J. An efficient method for the acetalisation of α,β-unsaturated aldehydes. J. Org. Chem., 1995, 60, 2931-2934.
2. Hayat, S.; Atta-ur-Rahman; Choudary, M. I.; Khan, K. M.; Bayer, E. An improved method for the synthesis of γ-lactones using sodium bromate and sodium hydrogen sulfite. Tetrahedron Lett., 2001, 42, 1647-1649.
3. Daily, R. D.; Dodge, J. A.; Hummel, C. W.; Jones, S. A.; Shepherd, T. A.; Wallace, O. B.; Weber, W. W. Selective estrogen receptor modulators. Patent WO 2005073205 A1, Aug. 11, 2005.
4. Masui, K.; Ikegami, H.; Mori, A. Palladium-catalyzed C—H homocoupling of thiophenes: facile construction of bithiophene structure. J. Am. Chem. Soc., 2004, 126, 5074-5075.
5. Wenkert, E. and Goodwin, T. E. 4-Formyl-2-cyclohexenone derivatives. Synth. Commun., 1977, 7, 409-415.
6. Costa, S. P. G.; Batista, R. M. F.; Cardoso, P.; Belsley, M.; Raposo, M. M. M. 2-Arylthienyl-substituted 1,3-benzothiazoles as new nonlinear optical chromophores. Eur. J. Org. Chem., 2006, 3938-3946.
7. Hedaya, E. and Theodoropulos, S. The preparation and reactions of stable phosphorus ylides derived from maleic anyhydrides, maleimides or isomaleimides. Tetrahedron, 1968, 24, 2241-2254.
8. Hutton, T. K.; Muir, K. W.; Proctor, D. J. Switching between novel samarium (II)-mediated cyclizations by a simple change in alcohol cosolvent. Org. Lett., 2003, 5, 4811-4814.
9. Tsuritani, T.; Kii, S.; Akao, A.; Sato, K.; Nonoyama, N.; Mase, T.; Yasuda, N. A short and efficient synthesis of isoindolin-1-ones. Synlett, 2006, 801-803.
10. Mori, M.; Kagoshima, Y.; Uchida, T.; Konosu, T.; Shibayama, T. Preparation of water-soluble triazole fungicides. Patent WO 2002066465 A1, 29 Aug. 2002.
11. Praveen, C.; Kumar, K. H.; Muralidharan, D.; Perumal, P. T. Oxidative cyclization of thiophenolic and phenolic Schiff's bases promoted by PCC: a new oxidant for 2-substituted benzothiazoles and benzoxazoles. Tetrahedron, 2008, 64, 2369-2374.
12. Dall'asta, L. and Cotticelli, G. Process for the preparation of 5-substituted isobenzofurans including citalopram. Patent EP 1281707 A1, 5 Feb. 2003.
13. Kumar, N.; Nayyer, S.; Mitu, G. Processes for the preparation of citalopram and its intermediate from 5-aminophthalide. Patent WO 2006103550 A1, 5 Oct. 2006.
14. Kobayashi, K.; Sugie, A.; Takahashi, M.; Masui, K.; Mori, A. Palladium-catalyzed coupling reactions of bromothiophenes at the C—H bond adjacent to the sulfur atom with a new activator system, AgNO₃/KF. Org. Lett., 2005, 7, 5083-5085.
15. Almansa Rosales, C. and Virgili Bemado, M. Bicylic derivatives as p38 kinase inhibitors. Patent WO 2007000339 A1, 4 Jan. 2007.
16. Biller, S. A. Aryl and heteroaryl (phosphinylmethyl) phosphonate squalene synthetase inhibitors and method. Patent EP 0570706 A1, 21 Apr. 1993.

1.-18. (canceled)

19. A method of inhibiting activity of a perforin molecule, or a fragment or variant thereof, on a cell, said method comprising exposing the cell to a benzylidene-2-thioxoimidazolidinone compound or a derivative thereof or a salt thereof.

20. A method of inhibiting activity of a perforin molecule, or a fragment or variant thereof, on a cell, said method comprising exposing the cell to a compound of the Formula (I): 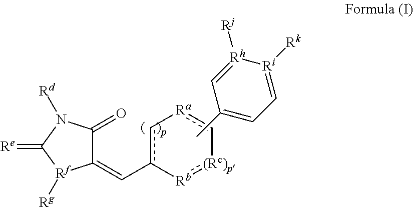
wherein:
R^{a}, R^{b} and R^{c} are independently C, N, S or O;
p and p′ are independently 0 or 1, provided that at least one of them is 1;
R^{d} is H, or methyl;
R^{e} is O, S, NH or absent;
R^{f} is C or N;
R^{g} is H or methyl;
R^{h} and Rⁱ are independently C or N;
R^{j} and R^{k} are independently H, F, Cl, Br, CN, Me, OMe, pyridyl, OH, SMe, acetyl, CH₂OAc, COOMe, COOEt, NHAc, NHSO₂Me, SO₂Me, SO₂NH₂, CONH₂, CONHJ or CONJJ;
or
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
—C(O)—Z—O—, where Z is NJ or NC(O)J; or
—OCO—; and
J is H or C₁-C₆ alkyl optionally substituted with acyl, hydroxyl, amino, alkylamino, alkenylamino, cycloalkylamino, cycloalkenylamino, arylamino, heteroaryl, heteroarylamino, heterocyclyl, heterocyclylamino, amino aryl amino, aminoheteroarylamino, aminoheterocyclylamino, alkylheterocyclyl, heteroarylcarbonylamino, heterocyclylcarbonylamino, alkyloxy, alkenyloxy, alkynyloxy, cycloalkyloxy, cycloalkenyloxy, aryloxy, heteroaryloxy, heterocyclyloxy, aminoalkyloxy, aminoalkenyloxy, aminoalkynyloxy, aminocycloalkyloxy, aminocycloalkenyloxy, aminoaryloxy, or aminoheteroaryloxy; or pharmaceutically acceptable salts, solvates, hydrates, and prodrugs thereof.

21. A method of inhibiting activity of a perforin molecule, or a fragment or variant thereof, on a cell, said method comprising exposing the cell to a compound of the Formula (Ia): 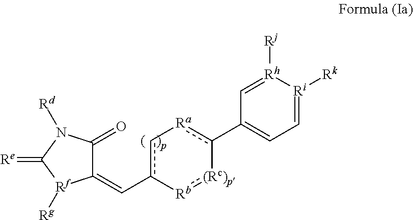
wherein:
R^{a}, R^{b} and R^{c} are independently C, N, S or O;
p and p′ are independently 0 or 1, provided that at least one of them is 1;
R^{d} is H or methyl;
R^{e} is O, S, NH or absent;
R^{f} is C or N;
R^{g} is H or methyl;
R^{h} and Rⁱ are independently C or N;
R^{j} and R^{k} are independently H, F, Cl, Br, CN, Me, OMe, pyridyl, OH, SMe, acetyl, CH₂OAc, COOMe, COOEt, NHAc, NHSO₂Me, SO₂Me, SO₂NH₂, CONH₂, CONHJ or CONJJ;
or
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
—C(O)—Z—O—, where Z is NJ or NC(O)J; or
—OCO—; and
J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof.

22. A method of inhibiting activity of a perforin molecule, or a fragment or variant thereof, on a cell, said method comprising exposing the cell to a compound of the Formula (II): 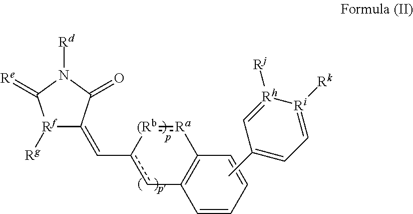
wherein:
R^{a} and R^{b} are independently C, N, S or O;
p and p′ are independently 0 or 1, provided that at least one of them is 1;
R^{d} is H or methyl;
R^{e} is O, S, NH or absent;
R^{f} is C or N;
R^{g} is H or methyl;
R^{h} and Rⁱ are independently C or N;
R^{j} and R^{k} are independently H, F, Cl, Br, CN, Me, OMe, pyridyl, OH, SMe, acetyl, CH₂OAc, COOMe, COOEt, NHAc, NHSO₂Me, SO₂Me, SO₂NH₂, CONH₂, CONHJ or CONJJ;
or
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
—C(O)—Z—O—, where Z is NJ or NC(O)J; or
—COC—; and
J is H or C₁-C₆ alkyl optionally substituted with acyl, hydroxyl, amino, alkylamino, alkenylamino, cycloalkylamino, cycloalkenylamino, arylamino, heteroaryl, heteroarylamino, heterocyclyl, heterocyclylamino, amino aryl amino, aminoheteroarylamino, aminoheterocyclylamino, alkylheterocyclyl, heteroarylcarbonylamino, heterocyclylcarbonylamino, alkyloxy, alkenyloxy, alkynyloxy, cycloalkyloxy, cycloalkenyloxy, aryloxy, heteroaryloxy, heterocyclyloxy, aminoalkyloxy, aminoalkenyloxy, aminoalkynyloxy, aminocycloalkyloxy, aminocycloalkenyloxy, aminoaryloxy, or aminoheteroaryloxy; or pharmaceutically acceptable salts, solvates, hydrates, and prodrugs thereof.

23. A compound of the formula (I): 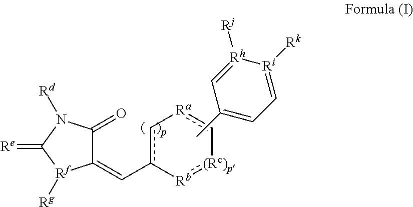
wherein:
R^{a}, R^{b} and R^{c} are independently C, N, S or O;
p and p′ are independently 0 or 1, provided that at least one of them is 1;
R^{d} is H or methyl;
R^{e} is O, S, NH or absent;
R^{f} is C or N;
R^{b} is H or methyl;
R^{h} and Rⁱ are independently C or N;
R^{j} and R^{k} are independently H, F, Cl, Br, CN, OMe, pyridyl, OH, SMe, acetyl, CH₂OAc, COOMe, COOEt, NHAc, NHSO₂Me, SO₂Me, SO₂NH₂, CONH₂, CONHJ or CONJJ, provided that when R^{j} is H R^{k} is not H;
or
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
—C(O)—Z—O—, where Z is NJ or NC(O)J; and
J is H or C₁-C₆ alkyl optionally substituted with acyl, hydroxyl, amino, alkylamino, alkenylamino, cycloalkylamino, cycloalkenylamino, arylamino, heteroaryl, heteroarylamino, heterocyclyl, heterocyclylamino, amino aryl amino, aminoheteroarylamino, aminoheterocyclylamino, alkylheterocyclyl, heteroarylcarbonylamino, heterocyclylcarbonylamino, alkyloxy, alkenyloxy, alkynyloxy, cycloalkyloxy, cycloalkenyloxy, aryloxy, heteroaryloxy, heterocyclyloxy, aminoalkyloxy, aminoalkenyloxy, aminoalkynyloxy, aminocycloalkyloxy, aminocycloalkenyloxy, aminoaryloxy, or aminoheteroaryloxy; or pharmaceutically acceptable salts, solvates, hydrates, and prodrugs thereof; provided that when R^{j} and R^{k} combined are a 5-membered ring of the formula —C(O)—Z—(CR^{l}R^{m})ₙ—, where n=1, Z is O and R^{l} and R^{m} are both H, R^{a}, R^{b} or R^{c} are not O.

24. A compound of the formula (Ia): 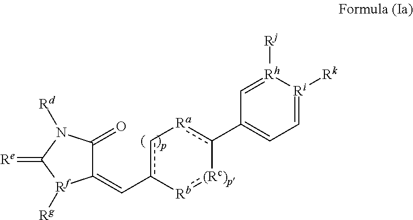
wherein:
R^{a}, R^{b} and R^{c} are independently C, N, S or O;
p and p′ are independently 0 or 1, provided that at least one of them is 1;
R^{d} is H or methyl;
R^{e} is O, S, NH or absent;
R^{f} is C or N;
R^{g} is H or methyl;
R^{h} and Rⁱ are independently C or N;
R^{j} and R^{k} are independently H, F, Cl, Br, CN, OMe, pyridyl, OH, SMe, acetyl, CH₂OAc, COOMe, COOEt, NHAc, NHSO₂Me, SO₂Me, SO₂NH₂, CONH₂, CONHJ or CONJJ, provided that when R¹ is H R² is not H;
or
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
—C(O)—Z—O—, where Z is NJ or NC(O)J; and
J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof; provided that when RJ and R^{k} combined are a 5-membered ring of the formula —C(O)—Z—(CR^{l}R^{m})ₙ—, where n=1, Z is O and R^{l} and R^{m} are both H, R^{a}, R^{b} or R^{c} are not O.

25. A compound of the formula (Ia): 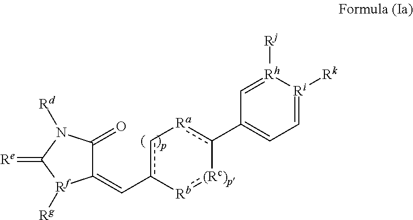
wherein:
R^{a}, R^{b} and R^{c} are independently C, N or S;
p and p′ are independently 0 or 1, provided that at least one of them is 1;
R^{d} is H or methyl;
R^{e} is O, S, NH or absent;
R^{f} is C or N;
R^{g} is H or methyl;
R^{h} and Rⁱ are independently C or N;
R^{j} and R^{k} are independently F, Cl, Br, CN, OMe, pyridyl, OH, SMe, acetyl, CH₂OAc, COOMe, COOEt, NHAc, NHSO₂Me, SO₂Me, SO₂NH₂, CONH₂, CONHJ or CONJJ;
or
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
—C(O)—Z—O—, where Z is NJ or NC(O)J; and
J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof.

26. A compound of the Formula Ia according to claim 25 wherein:
R^{a} and R^{b} are independently C, N or S;
R^{c} is C;
R^{d} is H;
R^{e} is O or S;
R^{f} is N;
R^{g} is H;
R^{h} and Rⁱ are C;
R^{j} and R^{k} combined are a 5-membered or 6-membered ring of the formula —C(O)—Z—(CR^{l}R^{m})ₙ— where n=1 or 2, Z is O, CH₂, NJ or NC(O)J, R^{l} and R^{m} are independently H, C₁-C₆ alkyl or cyclopropyl; or
C(O)—Z—O—, where Z is NJ or NC(O)J; and
J is H or C₁-C₆ alkyl optionally substituted with acyl, hydroxyl, amino, alkylamino, alkenylamino, cycloalkylamino, cycloalkenylamino, arylamino, aminoarylamino, morpholino, tetrahydropyridinylamino, azetidinyl, pyrrolidinyl, piperidinyl, piperazinyl, N-alkylpiperazinyl, azetidinylamino, pyrrolidinylamino, piperidinylamino, piperazinylamino, azetidinylcarbonylamino, pyrrolidinylcarbonylamino, piperidinylcarbonylamino, piperazinylcarbonylamino, alkyloxy, alkenyloxy, alkynyloxy, cycloalkyloxy, cycloalkenyloxy, aryloxy, aminoalkyloxy, aminoalkenyloxy, aminoalkynyloxy, aminocycloalkyloxy, aminocycloalkenyloxy, aminoaryloxy, aminoheteroaryloxy, azetidinyloxy, pyrrolidinyloxy, piperidinyloxy or piperazinyloxy; or pharmaceutically acceptable salts, solvates, hydrates, and prodrugs thereof.

27. A compound according to claim 26 wherein J is C₁-C₆ alkyl optionally substituted with acetyl, alkyloxy, alkylamino, morpholino, N-methylpiperazinyl or piperidinyl; or pharmaceutically acceptable salts, solvates, hydrates, and prodrugs thereof.

28. A compound of the formula I according to claim 23 selected from the following:
(E,Z)-5-((5-(1-Oxo-1,3-dihydroisobenzofuran-5-yl)thiophen-2-yl)methylene)-2-thioxoimidazolidin-4-one (2);
(E,Z)-5-((5-(1-Oxo-1,3-dihydroisobenzofuran-5-yl)thiophen-2-yl)methylene)imidazolidine-2,4-dione (3);
(E,Z)-2-Imino-1-methyl-5-((5-(1-oxo-1,3-dihydroisobenzofuran-5-yl)thiophen-2-yl)methylene)imidazolidin-4-one (4);
(E,Z)-5-(3-(1-Oxo-1,3-dihydroisobenzofuran-5-yl)benzylidene)-2-thioxoimidazolidin-4-one (5);
(E,Z)-5-(4-(1-Oxo-1,3-dihydroisobenzofuran-5-yl)benzylidene)-2-thioxoimidazolidin-4-one (6);
(E,Z)-5-((5-(1-oxo-1,3-dihydroisobenzofuran-5-yl)pyridin-2-yl)methylene)-2-thioxoimidazolidin-4-one (7);
(E,Z)-5-((5-(1-oxo-1,3-dihydroisobenzofuran-5-yl)pyridin-2-yl)methylene)-2-thioxoimidazolidin-4-one (8);
(E,Z)-5-((4-(1-Oxo-1,3-dihydroisobenzofuran-5-yl)thiophen-2-yl)methylene)-2-thioxoimidazolidin-4-one (15);
(E,Z)-4-(5-((5-Oxo-2-thioxoimidazolidin-4-ylidene)methyl)thiophen-2-yl)benzamide (16);
(E,Z)-3-(5-((5-Oxo-2-thioxoimidazolidin-4-ylidene)methyl)thiophen-2-yl)benzamide (17);
(E,Z)-4-(5-((5-Oxo-2-thioxoimidazolidin-4-ylidene)methyl)thiophen-2-yl)benzyl acetate (18);
(E,Z)-Methyl 4-(5-((5-oxo-2-thioxoimidazolidin-4-ylidene)methyl)thiophen-2-yl)benzoate (19);
(E,Z)-5-((5-(4-Acetylphenyl)thiophen-2-yl)methylene)-2-thioxoimidazolidin-4-one (20);
(E,Z)-Methyl 2-methyl-4-(5-((5-oxo-2-thioxoimidazolidin-4-ylidene)methyl)thiophen-2-yl)benzoate (21);
(E,Z)-5-((5-(4-Methoxyphenyl)thiophen-2-yl)methylene)-2-thioxoimidazolidin-4-one (22);
(E,Z)-5-((5-(3-Chlorophenyl)thiophen-2-yl)methylene)-2-thioxoimidazolidin-4-one (23);
(E,Z)-5-((5-(4-Chlorophenyl)thiophen-2-yl)methylene)-2-thioxoimidazolidin-4-one (24);
(E,Z)-5-((5-(Pyridin-4-yl)thiophen-2-yl)methylene)-2-thioxoimidazolidin-4-one (25);
(E,Z)-5-((5-(Pyridin-3-yl)thiophen-2-yl)methylene)-2-thioxoimidazolidin-4-one (26);
((E,Z)-5-((5-(4-(Methylthio)phenyl)thiophen-2-yl)methylene)-2-thioxoimidazolidin-4-one (27);
(E,Z)-5-((5-(4-(Methylsulfonyl)phenyl)thiophen-2-yl)methylene)-2-thioxoimidazolidin-4-one (28);
(E,Z)-5-((5-(4-Fluorophenyl)thiophen-2-yl)methylene)-2-thioxoimidazolidin-4-one (29);
(E,Z)-5-((5-(3,4-Difluorophenyl)thiophen-2-yl)methylene)-2-thioxoimidazolidin-4-one (30);
(E,Z)-5-((5-(3-Fluorophenyl)thiophen-2-yl)methylene)-2-thioxoimidazolidin-4-one (31);
(E,Z)-5-((5-(4-Bromophenyl)thiophen-2-yl)methylene)-2-thioxoimidazolidin-4-one (32);
(E,Z)-N-(4-(5-((5-Oxo-2-thioxoimidazolidin-4-ylidene)methyl)thiophen-2-yl)phenyl)methanesulfonamide (33);
(E,Z)-3-(5-((5-Oxo-2-thioxoimidazolidin-4-ylidene)methyl)thiophen-2-yl)benzonitrile (34);
(E,Z)-5-((5-(4-Hydroxyphenyl)thiophen-2-yl)methylene)-2-thioxoimidazolidin-4-one (35);
(E,Z)-3-((5-(1-Oxo-1,3-dihydroisobenzofuran-5-yl)thiophen-2-yl)methylene)pyrrolidine-2,5-dione (36);
(E,Z)-3-((5-(1-Oxo-1,3-dihydroisobenzofuran-5-yl)thiophen-2-yl)methylene)pyrrolidin-2-one (37);
(E,Z)-N-Methyl-4-(5-((5-oxo-2-thioxoimidazolidin-4-ylidene)methyl)thiophen-2-yl)benzamide (38);
(E,Z)-N,N-Dimethyl-4-(5-((5-oxo-2-thioxoimidazolidin-4-ylidene)methyl)thiophen-2-yl)benzamide (39);
(E,Z)-N-(2,3-Dihydroxypropyl)-4-(5-((5-oxo-2-thioxoimidazolidin-4-ylidene)methyl)thiophen-2-yl)benzamide (40);
(E,Z)-N-(2-Hydroxypropyl)-4-(5-((5-oxo-2-thioxoimidazolidin-4-ylidene)methyl)thiophen-2-yl)benzamide (41);
(E,Z)-N-(2-Hydroxyethyl)-4-(5-((5-oxo-2-thioxoimidazolidin-4-ylidene)methyl)thiophen-2-yl)benzamide (42);
(E,Z)-N-(3-Morpholinopropyl)-4-(5-((5-oxo-2-thioxoimidazolidin-4-ylidene)methyl)thiophen-2-yl)benzamide (43);
(E,Z)-N-(3-Hydroxypropyl)-4-(5-((5-oxo-2-thioxoimidazolidin-4-ylidene)methyl)thiophen-2-yl)benzamide (44);
(E,Z)-N-(2-Morpholinoethyl)-4-(5-((5-oxo-2-thioxoimidazolidin-4-ylidene)methyl)thiophen-2-yl)benzamide (45);
(E,Z)-5-(5-((5-Oxo-2-thioxoimidazolidin-4-ylidene)methyl)thiophen-2-yl)isoindolin-1-one (46);
(E,Z)-5-((5-(1-Oxoisoindolin-5-yl)thiophen-2-yl)methylene)imidazolidine-2,4-dione (47);
(E,Z)-5-(4-((5-Oxo-2-thioxoimidazolidin-4-ylidene)methyl)phenyl)isoindolin-1-one (48);
(E,Z)-5-(3-((5-Oxo-2-thioxoimidazolidin-4-ylidene)methyl)phenyl)isoindolin-1-one (49);
(E,Z)-5-(5-((5-Oxo-2-thioxoimidazolidin-4-ylidene)methyl)thiophen-3-yl)isoindolin-1-one (50);
(E,Z)-5-(6-((5-Oxo-2-thioxoimidazolidin-4-ylidene)methyl)pyridin-3-yl)isoindolin-1-one (51);
(E,Z)-2-Isopropyl-5-(5-((5-oxo-2-thioxoimidazolidin-4-ylidene)methyl)thiophen-2-yl)isoindolin-1-one (52);
(E,Z)-5-((5-(1-Oxo-1,3-dihydroisobenzofuran-5-yl)-1H-pyrrol-2-yl)methylene)-2-thioxoimidazolidin-4-one (53);
(E,Z)-5-((2-(1-Oxo-1,3-dihydroisobenzofuran-5-yl)oxazol-5-yl)methylene)-2-thioxoimidazolidin-4-one (54);
(E,Z)-5-((2-(1-Oxo-1,3-dihydroisobenzofuran-5-yl)thiazol-5-yl)methylene)-2-thioxoimidazolidin-4-one (55);
(E,Z)-5-((4-(1-Oxo-1,3-dihydroisobenzofuran-5-yl)thiazol-2-yl)methylene)-2-thioxoimidazolidin-4-one (56);
(E,Z)-5-((2-(1-Oxo-1,3-dihydroisobenzofuran-5-yl)thiazol-4-yl)methylene)-2-thioxoimidazolidin-4-one (57);
(E,Z)-5-((3-(1-Oxo-1,3-dihydroisobenzofuran-5-yl)isoxazol-5-yl)methylene)-2-thioxoimidazolidin-4-one (58);
(E,Z)-5-((3-(1-Oxo-1,3-dihydroisobenzofuran-5-yl)isothiazol-4-yl)methylene)-2-thioxoimidazolidin-4-one (59);
(E,Z)-5-((3-(1-Oxo-1,3-dihydroisobenzofuran-5-yl)isothiazol-5-yl)methylene)-2-thioxoimidazolidin-4-one (60);
(E,Z)-2-Methyl-5-(5-((5-oxo-2-thioxoimidazolidin-4-ylidene)methyl)thiophen-2-yl)isoindolin-1-one (61);
(E,Z)-2-Ethyl-5-(5-((5-oxo-2-thioxoimidazolidin-4-ylidene)methyl)thiophen-2-yl)isoindolin-1-one (62);
(E,Z)-5-(5-((5-Oxo-2-thioxoimidazolidin-4-ylidene)methyl)thiophen-2-yl)-2-propylisoindolin-1-one (63);
(E,Z)-2-Butyl-5-(5-((5-oxo-2-thioxoimidazolidin-4-ylidene)methyl)thiophen-2-yl)isoindolin-1-one (64);
(E,Z)-Ethyl 1-oxo-5-(5-((5-oxo-2-thioxoimidazolidin-4-ylidene)methyl)thiophen-2-yl)isoindoline-2-carboxylate (65);
(E,Z)-2-Acetyl-5-(5-((5-oxo-2-thioxoimidazolidin-4-ylidene)methyl)thiophen-2-yl)isoindolin-1-one (66);
(E,Z)-2-Acetyl-5-(5-((5-oxo-2-thioxoimidazolidin-4-ylidene)methyl)thiophen-2-yl)isoindolin-1-one (67);
(E,Z)-2-(5-(5-((2,5-Dioxoimidazolidin-4-ylidene)methyl)thiophen-2-yl)-1-oxoisoindolin-2-yl)ethyl acetate (68);
(E,Z)-2,3-Dimethyl-5-(5-((5-oxo-2-thioxoimidazolidin-4-ylidene)methyl)thiophen-2-yl)isoindolin-1-one (69);
(E,Z)-2-(2-Hydroxyethyl)-5-(5-((5-oxo-2-thioxoimidazolidin-4-ylidene)methyl)thiophen-2-yl)isoindolin-1-one (70);
(E,Z)-2-(3-Hydroxypropyl)-5-(5-((5-oxo-2-thioxoimidazolidin-4-ylidene)methyl)thiophen-2-yl)isoindolin-1-one (71);
(E,Z)-6-(5-((5-Oxo-2-thioxoimidazolidin-4-ylidene)methyl)thiophen-2-yl)isoindolin-1-one (72);
(E,Z)-2-Methyl-6-(5-((5-oxo-2-thioxoimidazolidin-4-ylidene)methyl)thiophen-2-yl)isoindolin-1-one (73);
(E)-2-(3-(Dimethylamino)propyl)-5-(5-((5-oxo-2-thioxoimidazolidin-4-ylidene)methyl)thiophen-2-yl)isoindolin-1-one (74);
(E,Z)-5-(5-((5-Oxo-2-thioxoimidazolidin-4-ylidene)methyl)thiophen-2-yl)-2-(3-(piperidin-1-yl)propyl)isoindolin-1-one (75);
(E,Z)-2-(3-(4-Methylpiperazin-1-yl)propyl)-5-(5-((5-oxo-2-thioxoimidazolidin-4-ylidene)methyl)thiophen-2-yl)isoindolin-1-one (76);
(E,Z)-5-(5-((5-Oxo-2-thioxoimidazolidin-4-ylidene)methyl)thiophen-2-yl)-2-(3-(pyrrolidin-1-yl)propyl)isoindolin-1-one (77);
(E,Z)-7-(5-((5-Oxo-2-thioxoimidazolidin-4-ylidene)methyl)thiophen-2-yl)-3,4-dihydroisoquinolin-1(2H)-one (81);
(E,Z)-6-(5-((5-Oxo-2-thioxoimidazolidin-4-ylidene)methyl)thiophen-2-yl)-3,4-dihydroisoquinolin-1(2H)-one (82);
(E,Z)-2-Methyl-7-(5-((5-oxo-2-thioxoimidazolidin-4-ylidene)methyl)thiophen-2-yl)-3,4-dihydroisoquinolin-1(2H)-one (83); and
(E,Z)-2-Methyl-6-(5-((5-oxo-2-thioxoimidazolidin-4-ylidene)methyl)thiophen-2-yl)-3,4-dihydroisoquinolin-1(2H)-one (84).

29. A compound of the formula I according to claim 23 selected from the following:
(E,Z)-5-((5-(1-Oxo-1,3-dihydroisobenzofuran-5-yl)thiophen-2-yl)methylene)-2-thioxoimidazolidin-4-one (2);
(E,Z)-5-((5-(1-Oxo-1,3-dihydroisobenzofuran-5-yl)thiophen-2-yl)methylene)imidazolidine-2,4-dione (3);
(E,Z)-2-Imino-1-methyl-5-((5-(1-oxo-1,3-dihydroisobenzofuran-5-yl)thiophen-2-yl)methylene)imidazolidin-4-one (4);
(E,Z)-5-(4-(1-Oxo-1,3-dihydroisobenzofuran-5-yl)benzylidene)-2-thioxoimidazolidin-4-one (6);
(E,Z)-5-((5-(1-oxo-1,3-dihydroisobenzofuran-5-yl)pyridin-2-yl)methylene)-2-thioxoimidazolidin-4-one (8);
(E,Z)-3-(5-((5-Oxo-2-thioxoimidazolidin-4-ylidene)methyl)thiophen-2-yl)benzamide (17);
(E,Z)-Methyl 4-(5-((5-oxo-2-thioxoimidazolidin-4-ylidene)methyl)thiophen-2-yl)benzoate (19);
((E,Z)-5-((5-(4-(Methylthio)phenyl)thiophen-2-yl)methylene)-2-thioxoimidazolidin-4-one (27);
(E,Z)-5-((5-(4-Bromophenyl)thiophen-2-yl)methylene)-2-thioxoimidazolidin-4-one (32);
(E,Z)-5-((5-(1-Oxoisoindolin-5-yl)thiophen-2-yl)methylene)imidazolidine-2,4-dione (47);
(E,Z)-5-(6-((5-Oxo-2-thioxoimidazolidin-4-ylidene)methyl)pyridin-3-yl)isoindolin-1-one (51);
(E,Z)-2-Isopropyl-5-(5-((5-oxo-2-thioxoimidazolidin-4-ylidene)methyl)thiophen-2-yl)isoindolin-1-one (52);
(E,Z)-5-((2-(1-Oxo-1,3-dihydroisobenzofuran-5-yl)thiazol-5-yl)methylene)-2-thioxoimidazolidin-4-one (55);
(E,Z)-5-((4-(1-Oxo-1,3-dihydroisobenzofuran-5-yl)thiazol-2-yl)methylene)-2-thioxoimidazolidin-4-one (56);
(E,Z)-5-((2-(1-Oxo-1,3-dihydroisobenzofuran-5-yl)thiazol-4-yl)methylene)-2-thioxoimidazolidin-4-one (57);
(E,Z)-5-((3-(1-Oxo-1,3-dihydroisobenzofuran-5-yl)isoxazol-5-yl)methylene)-2-thioxoimidazolidin-4-one (58);
(E,Z)-5-((3-(1-Oxo-1,3-dihydroisobenzofuran-5-yl)isothiazol-4-yl)methylene)-2-thioxoimidazolidin-4-one (59);
(E,Z)-2-Butyl-5-(5-((5-oxo-2-thioxoimidazolidin-4-ylidene)methyl)thiophen-2-yl)isoindolin-1-one (64);
(E,Z)-Ethyl 1-oxo-5-(5-((5-oxo-2-thioxoimidazolidin-4-ylidene)methyl)thiophen-2-yl)isoindoline-2-carboxylate (65);
(E,Z)-2-Acetyl-5-(5-((5-oxo-2-thioxoimidazolidin-4-ylidene)methyl)thiophen-2-yl)isoindolin-1-one (66);
(E,Z)-2-Acetyl-5-(5-((5-oxo-2-thioxoimidazolidin-4-ylidene)methyl)thiophen-2-yl)isoindolin-1-one, (67);
(E,Z)-2-(2-Hydroxyethyl)-5-(5-((5-oxo-2-thioxoimidazolidin-4-ylidene)methyl)thiophen-2-yl)isoindolin-1-one (70);
(E,Z)-2-(3-Hydroxypropyl)-5-(5-((5-oxo-2-thioxoimidazolidin-4-ylidene)methyl)thiophen-2-yl)isoindolin-1-one (71); and
(E,Z)-7-(5-((5-Oxo-2-thioxoimidazolidin-4-ylidene)methyl)thiophen-2-yl)-3,4-dihydroisoquinolin-1(2H)-one (81).

30. A pharmaceutical composition comprising a compound according to claim 23 together with a pharmaceutically acceptable carrier, excipient, diluent or adjuvant, or combinations thereof.

31. A method of treating or preventing a disease or disorder associated with undesirable perforin activity comprising administration of a therapeutically effective amount of a compound according to claim 23 to a subject in need thereof.

32. A method according to claim 31 wherein the disease or disorder is juvenile diabetes mellitus (type 1 or insulin dependant), graft-versus-host disease, or chronic or acute allograft rejection.

33. A method according to claim 31 wherein the disease or disorder is associated with cytotoxic T lymphocyte-mediated immune pathology.

34. A method according to claim 31 wherein the disease or disorder is perforin-induced immune pathology associated with virus infections.
